(19) United States
(12) Patent Application Publication
(10) Pub. No.: US 2019/0256822 A1
(43) Pub. Date: Aug. 22, 2019

(54) HEPATIC CELL LINE RESISTANT TO DIMETHYL SULFOXIDE, CELL CULTURE AND USES THEREOF

(71) Applicant: Academisch Medisch Centrum, Amsterdam (NL)

(72) Inventors: Vincent Alexander VAN DER MARK, Uithoorn (NL); Ruurdtje HOEKSTRA, Weesp (NL)

(21) Appl. No.: 16/312,772

(22) PCT Filed: Jul. 1, 2017
(86) PCT No.: PCT/EP2017/066405

§ 371 Date: Dec. 21, 2018

(30) Foreign Application Priority Data
Jul. 1, 2016 (EP) 16177623.2

(51) Int. Cl. C12N 5/071; C12N 15/85; C07K 14/705

(52) U.S. Cl. CPC C12N 5/0671; C12N 15/85; C12N 2503/04; C12N 2510/00; C12N 2513/00; C07K 14/70567

(57) ABSTRACT

The present invention relates to genetically modified HepaRG cells as deposited on Oct. 5, 2016 at the Leibniz-Institut DSMZ Deutsche Sammlung von Mikroorganismen and Zellkulturen GmbH, under No. DSM ACC3291. The invention further relates to methods of culturing said cells and cell cultures comprising said cells. The invention further relates to uses of the genetically modified HepaRG cells.

### TECHNICAL FIELD

The present invention relates to genetically modified HepaRG cells to improve their liver functionality and to methods producing said cells. The invention further relates to methods of culturing said cells and cell cultures comprising said cells. The invention further relates to uses of the genetically modified HepaRG cells.

### BACKGROUND

The pharmaceutical industry is committed to marketing safer drugs with fewer side effects, predictable pharmacokinetic properties and quantifiable drug-drug interactions. Drug-induced liver injury is the most frequent reason for withdrawing new candidates in later drug development stages and in post-marketed drugs. Occurrence of drug-induced hepatotoxicity is attributable to the poor predictability of preclinical animal studies. There are several reasons for this, including differences in drug metabolism between human and experimental species. These metabolic differences are of vital importance as hepatotoxicity is often associated with drug activation into reactive metabolites by drug-metabolising enzymes. Early detection of hepatotoxicity before testing compounds in animals and entering them in clinical trials is essential to save time and resources. Consequently, to improve and accelerate the lead identification and optimisation process, high-/medium-throughput cell-based assays have been incorporated into early drug development phases.

Primary cultured human hepatocytes (PHHs) are the “gold standard” for drug metabolism and hepatotoxicity studies. They are differentiated cells that express many hepatic functions, including drug metabolism. However their phenotypic instability, their scarcity, the irregular availability of liver tissue for cell harvesting, the poor plateability of certain lots of cryopreserved hepatocytes and the high variability of hepatic functions in hepatocytes obtained from different donors, and their lack of in vitro proliferation capacity, are drawbacks for their routine use in drug biotransformation and hepatotoxicity assessment.

To circumvent these problems, several proliferative hepatic cellular models have been developed. Cell lines derived from hepatocarcinomas present major advantages given their availability, easy handling, stable phenotype and unlimited life span. Other alternatives have also been explored in past years, including immortalisation of adult or foetal human hepatic cells by means of transformation with plasmids or viral vectors encoding immortalising genes, and hepatocytes differentiated from stem cells (from embryonic, mesenchymal or adult liver origin) or induced pluripotent stem cells or reprogrammed cells. Unfortunately, the majority of the human hepatocytes derived from these sources contains very low levels of drug metabolising enzymes, particularly cytochrome P450 (CYP) enzymes, as compared with primary human hepatocytes. Similarly, the expression of phase II-conjugating enzymes, among others N-acetyltransferase or GSH-transferase (GST) isoforms, do not resemble that of PHH. Basically, drug metabolism studies in HepG2, the most widely used human hepatoma cell line, are limited to those involving CYP1A or GST enzymes.

Several factors, probably acting at the same time on the cell genome, are involved in the repression of CYP genes in hepatic cells. Accordingly, new strategies have been used to up-regulate the expression of drug-metabolising enzymes in liver-derived cell lines (i.e., transfection with expression vectors encoding key hepatic transcription factors), indicating that tailored re-expression of these factors may be used to achieve significant re-activation of relevant CYPs. Alternatively, genetically engineered cell systems based on the stable or transient transfection of cells with gene/cDNAs encoding for individual drug metabolising enzymes provide tools for drug hepatotoxicity and metabolism testing. Yet, all these systems have not yet yielded a proliferative cell source that shows a full coverage of functions related to hepatic drug metabolism, which is highly needed for drug safety and metabolism studies.

In addition all these cell sources show an energy metabolism that is highly different from that of PHH; PHH depend for their energy on oxidative phosphorylation, which takes place in the mitochondria, and eliminate lactate, whereas proliferative cell sources show an energy metabolism mainly depending on glycolysis, which leads to the accumulation of lactate, even in presence of oxygen, and contain a limited amount of mitochondria (Nibourg et al., Exp Opin. Biol Ther. 2011; 43:1483-1489). This phenomenon is known as the Warburg effect, and is also a feature of tumor cells, as a means of rapid cytosolic energy production (VanderHeiden et al., Science 2009; 324:1029-1033 & Hirschhaeuser et al., Cancer Res 2011; 71; 6921-6925). It is highly conceivable that this feature renders the currently available proliferative cell sources for hepatocytes unsuitable for detecting mitochondrial toxicity, which may also be elicited by drugs.

The hepatic progenitor cell line HepaRG is used as alternative to PHHs for some in vitro applications. When fully differentiated, the culture consists of a mixture of hepatocyte islands and cholangiocyte-like cells (Gripon, Rumin et al. 2002, Cerec, Glaise et al. 2007). HepaRG cells possess high hepatic functionalities compared to PHHs and have been characterized as a useful model for drug metabolism studies (Aninat, Piton et al. 2006, Kanebratt and Andersson 2008, Kanebratt and Andersson 2008, Andersson, Kanebratt et al. 2012, Nibourg, Chamuleau et al. 2012b). HepaRG cells are suitable for predicting the human intrinsic clearance of many different drugs (Zanelli, Caradonna et al. 2012). Their long-term culture makes it possible to predict intrinsic clearance (CLint) of slowly metabolizable compounds, however, a recent study showed that the rate and prediction of low-clearance substances in HepaRG cells is still suboptimal (Bonn, Svanberg et al. 2016).

A disadvantage of HepaRG cells is that cultures of HepaRG cells lack certain differentiated cells which are present in fresh primary hepatocyte PHH. Differentiated HepaRG cells have higher levels of CYPs and of the transcripts of key nuclear factors controlling the expression of drug-metabolising enzymes (AhR, PXR, CAR) than any other commonly used hepatoma cell lines or other proliferative sources of human hepatocytes. If, however, they are compared with PHHs, the expression of CYPs in HepaRG cells is generally lower.

Another disadvantage of Human hepatoma HepaRG cells is that they require the treatment with dimethyl sulfoxide (DMSO) in order to differentiate towards a hepatocyte-like phenotype that is more suitable for drug metabolism studies. However, DMSO also induces cell death and may interfere with cellular activities. Unmodified, differentiated HepaRG cells seem to have normal CAR signaling, since they mimic ligand-stimulated CAR trafficking from the cytoplasm to the nucleus as observed in PHHs (: Jackson, Li, Chamberlain, Wang, Ferguson, D M D 2016 Jun. 23). However, the level of CAR expression in fully differentiated HepaRG cells is 5- to 10-fold lower when compared to PHHs, and requires the addition of DMSO during the differentiation phase (Aninat, Piton et al. 2006). In HepaRG cells the high expression of drug metabolizing enzymes and xenobiotic receptors other than CAR is also dependent on the addition of DMSO (Aninat, Piton et al. 2006, Kanebratt and Andersson 2008). DMSO is commonly used in hepatic cell cultures to induce and maintain liver-specific differentiation by unknown mechanisms (Higgins and Borenfreund 1980, Villa, Arioli et al. 1991, Yu and Quinn 1994, Paran, Geiger et al. 2001, Azuma, Hirose et al. 2003, Aninat, Piton et al. 2006, Sainz and Chisari 2006, Choi, Sainz et al. 2009). However, DMSO concentrations as low as 0.1% can also have a significant negative effect on the activity of phase I drug metabolizing enzymes (Chauret, Gauthier et al. 1998, Busby, Ackermann et al. 1999, Gonzalez-Perez, Connolly et al. 2012). It is well known that long-term dosing and high concentrations of DMSO reduce cell viability (Galvao, Davis et al. 2014). DMSO treatment of HepaRG monolayers reduces total cellular protein content by more than 50% and increases cell leakage 3- to 4-fold (Hoekstra, Nibourg et al. 2011). Moreover, DMSO treatment reduces the majority of hepatic functions of HepaRG cells unrelated to drug metabolism, particularly when cultured under optimal conditions, as supplied by a three-dimensional, oxygenated, and medium-perfusion environment in a bioartificial liver (Nibourg, Chamuleau et al. 2012a).

In addition, HepaRG cells cannot be cultured in serum free medium without utilization of expensive growth factors, making them less suitable for production of for instance blood factors. A potential benefit from serum-free culturing would be that the culture medium would comprise proteins that are solely produced by the cells. This raises the possibility that liver cells, being the producers of a large amount of blood proteins, could be exploited for the production of a mixture of these proteins. Currently these proteins are isolated for scientific, diagnostic, or therapeutic purposes from human plasma or from medium of recombinant non-hepatic cultures. The isolation from human plasma requires large plasma volumes, and holds the risks of transmission of known or unknown pathogens. The production in cell lines requires the use of serum, which is also associated with the risk of pathogen transmission, and additionally, the product may not be modified accurately after translation, resulting e.g. in an inactive product.

A further disadvantage is that HepaRG cells are difficult to infect with malaria parasites and are therefore less suitable for malaria research. Very recently, humanized mouse models have been developed that make it possible to study the full life cycles of the human malaria parasites P. falciparum and P. ovale (Soulard, Bosson-Vanga et al. 2015) and the liver stage development of P. vivax (Mikolajczak, Vaughan et al. 2015). However, in vitro models to study the liver stage of human malaria Plasmodium parasites are currently lacking. The human hepatoma cell line HC04 (Sattabongkot, Yimamnuaychoke et al. 2006) is the most commonly used liver cell line for studies on human malaria infection, but is considered to suboptimally model human hepatocyte biology, because of altered and/or reduced hepatic functionality, signaling, and gene expression patterns (March, Ng et al. 2013), and therefore makes it difficult to conduct detailed mechanistic, translational, or malaria drug screening studies. Interestingly, an improved micropatterned PHH/mouse fibroblast co-culture has been reported that effectively sustains the hepatic life cycle of P. falciparum and P. vivax (March, Ng et al. 2013, March, Ramanan et al. 2015). Although hepatocyte infection rates of malaria parasites in this system are in the range that can be attained in studies on HC04 cells, they are still very low (0.06-0.18%). In addition, as indicated above, PHH are limited in supply and expensive to obtain. Although HepaRG cells are easier to obtain and approach hepatic functionality of PHHs, infection of P. cynomolgi or P. falciparum could not be established in either primary simian or human hepatocytes co-cultured with HepaRG cells (Dembele, Franetich et al. 2014).

Another disadvantage is that HepaRG cells cannot be expanded without loss of functionaliy: The HepaRG cells phenotype remains stable for ˜20 passages, after which the cells lose their ability to differentiate (Laurent, Glaise et al. 2013). The supply of HepaRG cells is therefore finite and—relevant for application-batch sizes are limited by the maximum number of passages. With the currently applied split ratio of 1:5 during each passage, the cells can be expanded theoretically until 5²⁰ cells.

However, in practice this number will be substantially lower, due to yield of cells of passage number lower than 20 for experimentation and application purposes.

A further disadvantage of HepaRG cells is that the cells cannot be preserved short-term (24 hours) at low temperature (4° C.) without loss of functionality. This limits the rapid utilization of fully differentiated HepaRG cells when transported to the end-user under conditions with limited oxygen and culture medium supply, but with high cell quantity, as found for HepaRG cells that are fully matured in a bioreactor, like a bioartificial liver. For instance, the AMC-bioartificial liver housing matured HepaRG cells, which has been developed to supply liver support to patients with liver failure (Nibourg, Chamuleau et al. 2012a). A 24-hour preservation of laboratory-scale AMC-BALs at 20° C. with active oxygenation already showed a significantly decreased lactate elimination (72%), ammonia elimination (40%) and apolipoprotein A1 synthesis (41%), as well as an increased leakage of Aspartate transaminase (265%), indicating cell death, compared to baseline BAL cultures maintained at 36° C. A 16-hour preservation at 4° C. with passive oxygenation decreased lactate elimination (95%), ammonia elimination (55%) and apolipoprotein A1 synthesis (45%).

It is therefore an object of the invention to provide a hepatic cell line which overcomes one or more of the above mentioned problems.

### SUMMARY OF THE INVENTION

The invention is based on the finding that a genetic modification in the genome of human liver cell line HepaRG, resulting in the overexpression of the nuclear receptor NR1I3, also named constitutive androstane receptor (CAR), causes improved liver functionality. In particular, the inventors found that the drug metabolism was highly improved and surprisingly the energy metabolism became more similar to that of PHH, and HepaRG culturing could be performed in serum-free conditions both in the presence or absence of DMSO (see FIG. 8D). In addition the CAR overexpression increased P. falciparum infectivity. These effects were less present or absent after overexpressing CAR in HepG2 cells. Furthermore, the genetically modified HepaRG cells of the invention became more robust: the stability over passages was increased and the cells were less negatively affected by a 24-hour period of preservation at 4° C.

CAR is a nuclear receptor that is predominantly expressed in hepatocytes and is an established regulator of a vast array of genes involved in lipid homeostasis, cell proliferation, and drug, bilirubin, thyroid hormone and energy metabolism. (Huang, Zhang et al. 2004, Maglich, Watson et al. 2004, Gao and Xie 2010, Molnár, Küblbeck et al. 2013, Yang and Wang 2014). In PHHs CAR is present in the cytoplasm, where it translocates to the nucleus after ligand-dependent or independent activation (Kawamoto, Sueyoshi et al. 1999, Li, Chen et al. 2009, Mutoh, Sobhany et al. 2013). Here, it binds to response elements in regulatory regions of various genes as a heterodimer with the retinoid X receptor (RXR, NR2B) (reviewed in (Yang and Wang 2014)). Many endogenous and exogenous compounds, including steroid hormones, bile acids, and drugs have been identified as activators of CAR (Hernandez, Mota et al. 2009, Lynch, Zhao et al. 2015). However, CAR also has high transcriptional activity in the absence of any externally added ligand (Baes, Gulick et al. 1994, Choi, Chung et al. 1997).

By overexpressing CAR in the human liver cell line HepaRG, the inventors further demonstrated improved differentiation and increased resistance to the toxic effects of DMSO. In addition, the inventors have observed enhanced phase 1 and 2 drug metabolic activities after culture with or without DMSO. The inventors also showed an increased capability of these cells to metabolize the low clearance compounds warfarin and prednisolone. In conclusion, overexpression of CAR creates a more physiologically relevant environment for studies on hepatic (drug) metabolism and differentiation in HepaRG cells without the utilization of DMSO.

The inventors have compared HepaRG wherein CAR is overexpressed to HepG2 cells wherein CAR is overexpressed. Overexpression of CAR in HepG2 cells is described by Haishan Li et al., in Drug Metab Dispos, 2009 May; 37(5): 1098-1106. However, herein none of the surprising effects as listed above and described in more detail herein after, are mentioned.

Effects of CAR overexpression were less prominent on transcript levels of CAR target genes in drug metabolism in HepG2 cells. The HepG2 cell line is the most commonly used hepatoma cell line in biological research, but suffers from unrestricted growth because of overexpression of genes associated with cell cycle and checkpoint control (Jennen, Magkoufopoulou et al. 2010). This makes it difficult to culture HepG2 cells during a similar period as HepaRG cells (i.e. 4 weeks).

The inventors have also shown that overexpression of CAR in HepaRG cells not only increased phase 1 and phase 2 drug metabolism, but also induced the formation of more hepatocyte-like cells during differentiation with DMSO, decreased lactate production, increased mitochondrial DNA (mtDNA) content in combination with DMSO, and cellular NADH levels (measured by WST-1 activity), increased infectivity of human malaria parasites, and increased oxygen consumption. These effects were most prominent when the cells were cultured in a 3D environment with medium perfusion suitable for multi-well testing, called BAL-in-a-dish (BALIAD) culture.

Moreover, the effects on energy metabolism seemed largely absent in HepG2 cells. Despite a small increase in mtDNA content in DMSO cultured HepG2-CAR cells, other variables, such as lactate consumption, cellular NADH levels (measured by WST-1 activity), and total protein content after addition of DMSO were unaffected.

The invention therefore provides a modified HepaRG cell, wherein the modification induces overexpression of the human CAR protein in comparison with an unmodified HepaRG cell.

Preferably, said unmodified HepaRG cell has the same copy number of the CAR/NR1I3 gene as a HepaRG cells as deposited on 5 Apr. 2001 at the Collection Nationale de Cultures de Microorganismes, Institut Pasteur, under No. 1-2652.

The invention further provides a modified HepaRG cell, wherein the modification comprises an additional copy of the CAR/NR1I3 gene compared to the HepaRG cell as deposited on 5 Apr. 2001 at the Collection Nationale de Cultures de Microorganismes, Institut Pasteur, under No. 1-2652.

Preferably, the modification induces overexpression of the CAR/NR1I3 gene in comparison with the HepaRG cell as deposited on 5 Apr. 2001 at the Collection Nationale de Cultures de Microorganismes, Institut Pasteur, under No. 1-2652.

In a preferred embodiment, said overexpression is in an amount exceeding 100% of human control liver tissue, preferably 130%, preferably 200%, when cultured in DMSO.

In another preferred embodiment, said overexpression is at least 12 times, more preferably at least 20 times higher than in an unmodified HepaRG cell.

Preferably, said overexpression is in an amount which causes an increase in WST-1 activity by at least 30%, more preferably at least 40% in comparison to an unmodified HepaRG cell.

In another preferred embodiment, said overexpression is stable for at least 7 passages. Preferably, said modification comprises a copy of an exogenous CAR/NR1I3 gene. Preferably, said modified HepaRG cell comprises a nucleic acid construct, wherein said nucleic acid construct preferably comprises the Pgk-1 promoter.

In another preferred embodiment, said modified HepaRG cell is infected by a malaria parasite.

In a preferred embodiment, the modified HepaRG cell is as deposited on [Oct. 5, 2016] at the DSMZ, under No. DSM ACC3291.

The invention further provides a method of producing the modified HepaRG cell, comprising steps of: (a) providing a cell culture of HepaRG cells, (b) modifying the HepaRG cells using a nucleic acid construct comprising the CAR/NR1I3 gene and a selection marker, and (c) selecting a modified HepaRG cell using the selection marker.

In another aspect, the invention provides a method of culturing the modified HepaRG cell as defined above in a culture medium.

Preferably, the culturing is performed in a three-dimensional culture system.

In a preferred embodiment, the culture medium is substantially free of DMSO.

Preferably, the culture medium is substantially free of serum.

The invention further provides a modified HepaRG cell obtainable by the method according to the invention.

The invention further provides a cell culture comprising the modified HepaRG cell as defined above.

Preferably, said cell culture is characterized by the presence of hepatocyte-like cells in between the hepatocyte islands of more than 25% of the cells in said cell culture, preferably more than 30, 35, 40, 45, 50%.

The invention further provides use of the modified HepaRG cell according to the invention or the cell culture according to the invention in a method of determining clearance of a compound.

The invention further provides a method of producing a protein of interest comprising the steps of: (a) providing a cell culture of modified HepaRG cells, (b) allow the expression of the protein of interest, and (c) isolate the protein of interest.

In a preferred embodiment, step b. is performed in the absence of serum.

The invention further provides a method of infecting a modified HepaRG cell with a malaria parasite comprising steps of: providing a cell culture of modified HepaRG cells according to the invention and adding malaria parasites to the cell culture and allow the infection of the modified HepaRG cells.

### BRIEF DESCRIPTION OF THE DRAWINGS

FIG. 1 shows CAR overexpression and morphology of + and −DMSO monolayer cultures of HepaRG and HepG2 cells. FIG. 1(A) shows relative CAR transcript levels (average of 6 independent experiments of at least 3 separate samples in each group). ***p<0.001. FIG. 1(B) shows phase-contrast images. Black arrows indicate inter-island hepatocyte-like cells. Bar=60 μm. FIGS. 1(C) and (D) show immunofluorescence staining for albumin (C) or MRP2 (D). Bar=60 μm.

FIG. 2 shows the effect of CAR overexpression and DMSO treatment on the induction of xenobiotic nuclear receptors in HepaRG cells. FIG. 2(A) shows the fold increase in CYP1A2 transcript levels in HepaRG+/− CAR monolayer cultures after treatment with omeprazole. FIG. 2(B) shows the fold increase in CYP2B6 transcript levels in HepaRG+/− CAR monolayer cultures after treatment with CITCO. FIG. 2(C) shows the fold increase in CYP3A4 transcript levels in HepaRG+/− CAR monolayer cultures after treatment with rifampicin. FIG. 2(D) shows the fold increase in CYP1A2 transcript levels in HepaRG+/−CAR BALIAD cultures after treatment with omeprazole. FIG. 2(E) shows the fold increase in CYP2B6 transcript levels in HepaRG+/− CAR BALIAD cultures after treatment with CITCO. FIG. 2(F) shows the fold increase in CYP3A4 transcript levels in HepaRG+/− CAR BALIAD cultures after treatment with rifampicin. All figures represent the average of 1 (D-F) or 2 (A-C) experiments of at least 3 separate samples in each group. *p<0.05; ***p<0.001.

FIG. 3 shows the effect of CAR overexpression and DMSO treatment on CYP activity of HepaRG cells. FIG. 3(A) shows the CYP2B6 activity of HepaRG+/− CAR monolayer cultures. FIG. 3(B) shows the CYP2C9 activity of HepaRG+/−CAR monolayer cultures. FIG. 3(C) shows the CYP3A4 activity of HepaRG+/− CAR monolayer cultures. FIG. 3(D) shows the CYP1A2 activity of HepaRG+/−CAR monolayer cultures. FIG. 3(E) shows the CYP2D6 activity of HepaRG+/− CAR monolayer cultures. FIG. 3(F) shows the CYP2E1 activity of HepaRG+/− CAR monolayer cultures. FIG. 3(G) shows the CYP2B6 activity of HepaRG+/− CAR BALIAD cultures. FIG. 3(H) shows the CYP3A4 activity of HepaRG+/− CAR BALIAD cultures. All figures represent 1 experiment of at least 3 separate samples in each group. *p<0.05; **p<0.01; ***p<0.001.

FIG. 4 shows the effect of CAR overexpression and DMSO treatment on bilirubin glucuronidation activity of HepaRG monolayer cultures. FIG. 4(A) shows the bilirubin mono- and diglucuronidation activity of intact HepaRG+/−CAR cells, measured in culture medium. FIG. 4(B) shows the bilirubin mono- and diglucuronidation activity in cell homogenates of HepaRG+/−CAR cells. All figures represent the average of 2 independent experiments of at least 3 separate samples in each group. ***p<0.001.

FIG. 5 shows the effect of CAR overexpression and DMSO treatment on acetaminophen-, amiodarone-, indomethacin-, and dextromethorphan-induced toxicity in HepaRG monolayer cultures. It shows the total relative ATP levels of HepaRG+/− CAR cells after 24 h treatment with increasing concentrations of the indicated compounds. All figures represent the average of 2-3 independent experiments of 3 separate samples in each group.

FIG. 6 shows the effect of CAR overexpression and DMSO treatment on the clearance of warfarin, theophylline, and prednisolone in HepaRG monolayer cultures. FIG. 6(A) shows the levels of warfarin in culture medium of DMSO cultured HepaRG+/−CAR cells during 6 days. 1 experiment of 3 separate samples for each point. FIG. 6(B) shows the levels of warfarin and prednisolone in culture medium of HepaRG+/−CAR cells during 1 day. Figures in (B) represent the average of 3 (prednisolone) or 4 (warfarin) independent experiments of 3 separate samples in each group.

FIG. 7 shows the effect of CAR overexpression and DMSO treatment on albumin synthesis and ammonia elimination in monolayer cultures. FIG. 7(A) shows the albumin synthesis in HepaRG+/−CAR cells (1 experiment of 3 separate samples in each group). FIG. 7(B) shows the ammonia elimination in HepaRG+/−CAR (average of 4 independent experiments of at least 3 separate samples in each group) and HepG2+/−CAR (average of 2 independent experiments of at least 3 separate samples in each group) cells. *p<0.05; **p<0.01; ***p<0.001.

FIG. 8 shows the effect of CAR overexpression and DMSO treatment on total protein, viability and integrity of HepaRG and HepG2 monolayer cultures. FIG. 8(A) shows the total protein and decreased fraction of total protein due to DMSO treatment of HepaRG+/−CAR cells (average of 11 independent experiments of at least 3 separate samples in each group) and HepG2+/−CAR (average of 4 independent experiments of at least 3 separate samples in each group) cells. FIG. 8(B) shows the cellular AST leakage of HepaRG+/−CAR cells (1 experiment with 3 separate samples in each group). FIG. 8(C) shows the cellular ATP levels of HepaRG+/−CAR cells (1 experiment with 3 separate samples in each group). FIG. 8(D) shows the cellular WST-1 activity corrected for protein of HepaRG+/−CAR cells (average of 4 independent experiments of at least 3 separate samples in each group) and HepG2+/−CAR cells relative to unmodified non-DMSO treated cultures (average of 2 (DMSO−) or 3 (DMSO+) independent experiments of at least 3 separate samples in each group). * p<0.05; **p<0.01; ***p<0.001.

FIG. 9 shows the effect of CAR overexpression and DMSO treatment on energy metabolism of HepaRG and HepG2 monolayer cultures. FIG. 9(A) shows the glucose consumption of HepaRG+/− CAR (average of 4 independent experiments of at least 3 separate samples in each group) cells.

FIG. 9(B) shows the lactate production of HepaRG+/−CAR (average of 3 independent experiments of at least 3 separate samples in each group) and HepG2+/−CAR (average of 2 independent experiments of at least 3 separate samples in each group) cells. *p<0.05; ***p<0.001.

FIG. 10 shows the effect of CAR overexpression and DMSO treatment on mitochondrial DNA content of HepaRG and HepG2 cells. FIG. 10(A) shows the relative mitochondrial DNA/nuclear DNA ratios of HepaRG+/−CAR and HepG2+/−CAR monolayer cultures (average of minimally 2 independent experiments of at least 6 separate samples in each group). FIG. 10(B) shows the relative mitochondrial DNA/nuclear DNA ratios of HepaRG+/−CAR BALIAD cultures with monolayer control cells set to 1 (1 experiment of 3 separate samples in each group). FIG. 10(C) shows the oxygen consumption of HepaRG+/−CAR monolayer cultures measured in the Seahorse (average of 6 separate samples in each group). **p<0.01 ***p<0.001. FIG. 10(D) shows the oxygen consumption of HepaRG+/−CAR BALIAD cultures transferred to OxoPlates (average of 8 independent experiments of at least 3 separate samples in each group). **p<0.01 ***p<0.001.

FIG. 11 shows the effect of CAR overexpression and DMSO treatment on serum-free culture of HepaRG monolayer cultures after 28 days of conventional culture. FIG. 11(A) shows phase contrast images of HepaRG+/−CAR cells cultured without DMSO. FIG. 11(B) shows phase contrast images of HepaRG+/−CAR cells cultured with DMSO. Bar=60 μm.

FIG. 12 shows expression of Complement factor 6 and fibrinogen in culture medium of HepaRG and HepG2 cells. FIG. 12 shows Western blots of culture medium samples of serum-free cultures of HepaRG BALIAD+/−CAR and HepG2+/−C6 monolayer cells and of serum-free medium not exposed to cells. FIG. 12(A) demonstrates the detection of C6. FIG. 12(B) shows the detection of non-reduced fibrinogen. FIG. 12(C) shows the detection of reduced fibrinogen. Arrows indicate bands of the protein of interest. Molecular weight ladder is added in the first lane.

FIG. 13 shows the effect of CAR overexpression on infection of Plasmodium falciparum in HepaRG monolayer cultures. Cycle threshold (Ct) values are shown of P. falciparum 18S ribosomal RNA RT-qPCR of HepaRG+/−CAR cells 1, 3, or 5 days after infection with living sporozoites (+), killed sporozoites (D), or vehicle (−). This figure represents 1 experiment of 3 separate samples in each group.

FIG. 14 shows the effect of CAR overexpression on the stability of HepaRG cells during consecutive passaging. Indicated are HepaRG and HepaRG-CAR cells expanded under conventional conditions with medium containing 10% fetal bovine serum, and HepaRG-CAR cells expanded in culture medium with only 2.5% fetal bovine serum. FIG. 14(A) shows the total protein/cm2. FIG. 14(B) shows the Ammonia elimination. FIG. 14(C) shows the lactate production. For FIG. 14 A-C: average of minimally 2 independent experiments of 3 separate samples for control HepaRG from P15-P23 and for HepaRG-CAR at P18 and P23, in other cases 1 experiment of 3 separate samples. ***p<0.001 control cells vs CAR cells either maintained in 10% or 2.5% FBS-containing medium. FIG. 14(D) shows the morphology of HepaRG cells at passage 18 and 23 and of HepaRG cells at passage 33 expanded in culture medium with 2.5% of 10% fetal bovine serum. All cultures were terminally matured in culture medium with 10% fetal bovine serum.

FIG. 15 shows the effect of CAR overexpression on a 24-hour preservation period at 4° C. of HepaRG cells cultured in BALIADs, and treated with or without 5 mM N-acetylcysteine (NAC)+100 μM dopamine (DA) compared to cultures maintained at 37° C. (average of 2 independent experiments of 3 separate samples in each group). *p<0.05, **p<0.01.

FIG. 16 shows the effect of CAR overexpression in HepaRG monolayer cells on the reactive oxygen species, as measured by mitosox fluorescence (average of 2 independent experiments of 2 separate samples in each group). ***p<0.001.

FIG. 17 shows the effect of CAR overexpression and DMSO treatment on transcript levels of CYP1A2, CYP2B6, and CYP3A4 after induction of xenobiotic nuclear receptors in HepaRG cells.

FIG. 17(A) shows the relative CYP1A2, CYP2B6, or CYP3A4 transcript levels in HepaRG+/−CAR monolayer cultures after treatment with respectively omeprazole, CITCO, or rifampicin. FIG. 17(B) shows the relative CYP1A2, CYP2B6, or CYP3A4 transcript levels in HepaRG+/−CAR BALIAD cultures after treatment with respectively omeprazole, CITCO, or rifampicin. All figures represent the average of ½ experiments of at least 3 separate samples in each group. *p<0.05; **p<0.01; ***p<0.001.

FIG. 18 shows the effect of CAR overexpression and DMSO treatment on total protein and lactate metabolism in HepaRG cells. FIG. 18(A) shows the total protein of BALIAD cultured HepaRG+/−CAR cells with 0%, 0.85%, or 1.7% DMSO. **p<0.01; ***p<0.001 vs control 0% and CAR 0%. FIG. 18(B) shows the lactate production or consumption of monolayer and BALIAD cultured HepaRG+/−CAR cells. *p<0.05; **p<0.01. FIG. 18(C) shows lactate production or consumption of BALIAD cultured HepaRG+/−CAR cells with 0%, 0.85%, or 1.7% DMSO. *p<0.05; **p<0.01; ***p<0.001. All figures represent 1 experiment of at least 3 separate samples in each group, except the monolayer condition in FIG. 18(B) which represents 3 independent experiments of at least 3 separate samples in each group.

Table 1. The effect of CAR overexpression and DMSO treatment on transcript levels of HepaRG cells

Transcript levels of HepaRG+/−CAR and HepG2+/−CAR monolayer cultures cultured with or without DMSO, and HepaRG+/−CAR cells cultured in BALIAD as % of mean transcript levels of two human liver samples (n=1-6 independent experiments per gene, at least 3 separate samples per group).

Table 2. The effect of CAR overexpression and DMSO treatment on transcript levels of HepG2 monolayer cultures. Transcript levels of HepG2+/−CAR cells cultured with or without DMSO as % of mean transcript levels of two human liver samples (n=1-2 independent experiments per gene, at least 3 separate samples per group). *p<0.05; **p<0.01; ***p<0.001 vs HepG2 (DMSO−); ^{#}p<0.05; ^{##}p<0.01; ^{###}p<0.001 vs HepG2-CAR (DMSO−); ^{$}p<0.05; ^{$s}p<0.01; ^{$$$}p<0.001 vs HepG2 (DMSO+).

Table 3. Metabolism of amiodarone, acetaminophen, indomethacin, and dextromethorphan. Major metabolic routes, human Cₘₐₓ values and TC50 values of amiodarone, acetaminophen, indomethacin, and dextromethorphan in DMSO-treated HepaRG+/−CAR monolayer cultures are indicated. *p<0.05, ***p<0.001 vs HepaRG, ND=not determined.

Table 4 Elimination of low clearance compounds. Major metabolic routes and CLint [μl/min/10⁶ cells]±SD of prednisolone, warfarin, and theophylline in HepaRG+/−CAR monolayer cultures cultured without or with DMSO are indicated. No reliable clearance of theophylline could be detected. ***p<0.001 vs HepaRG (DMSO−); ^{##}=p<0.01; ^{###}=p<0.001 vs HepaRG-CAR (DMSO−); ^{$}=p<0.05; ^{$$$}=p<0.001 vs HepaRG (DMSO+), ND=not determined. Prednisolone, warfarin, and theophylline were determined respectively in 3, 4, and 1 independent experiments with 3 separate replicates each.

Table 5 The effect of CAR overexpression on transcript levels of HepaRG cells cultured in BALIAD. Transcript levels of HepaRG+/−CAR cells as % of mean transcript levels of two human liver samples (n=1-2 independent experiments per gene, at least 3 separate samples per group). *p<0.05; **p<0.01; ***p<0.001 vs HepaRG BALIAD.

### DETAILED DESCRIPTION

### Embodiments

Modified HepaRG Cells

The term “HepaRG cell” or “unmodified HepaRG cell” as used herein refers to a hepatocyte as described in Moffett J R, Namboodiri M A., loc. cit; Watanabe et al., “stereospecificity of hepatic 1-tryptophan 2,3-dioxygenase.” Biochem J, 1980. 189(3): p. 393-405; Knox and Mehler, “the adaptive increase of the tryptophan peroxidase-oxidase system of liver.” Science, 1951. 113(2931): p. 237-8; Liao et al., “impaired dexamethasone-mediated induction of tryptophan 2,3-dioxygenase in heme-deficient rat hepatocytes: translational control by a hepatic eif2alpha kinase, the heme-regulated inhibitor”. J Pharmacol Exp Ther. 2007 dec; 323(3):979-89). Preferably the HepaRG cell is as deposited on 5 Apr. 2001 at the Collection Nationale de Cultures de Microorganismes, Institut Pasteur, under No. 1-2652.

The term “modified HepaRG cell” as used herein refers to a HepaRG cell comprising a genetic modification, wherein said modification induces overexpression of the CAR/NR1I3 gene in comparison with an unmodified HepaRG cell.

The term “genetic modification” as used herein refers to the stable or transient alteration of the genotype of a cell by introduction of exogenous DNA. DNA may be synthetic, or naturally derived, and may contain genes, portions of genes, or other useful DNA sequences. The term “genetic modification” as used herein is not meant to include naturally occurring alterations such as that which occurs through natural viral activity, natural genetic recombination, or the like. Exogenous DNA may be introduced to a precursor cell by viral vectors (retrovirus, modified herpes viral, herpes-viral, adenovirus, adeno-associated virus, and the like) or direct DNA transfection (lipofection, calcium phosphate transfection, DEAE-dextran, electroporation, and the like).

As used herein, the term “overexpression” refers to a process by which a nucleic acid comprising a CAR/NR1I3 gene sequence that encodes CAR is artificially expressed in the modified cell to produce a level of expression of the transcript or the encoded polypeptide that exceeds the level of expression of the transcript or the encoded polypeptide in the unmodified HepaRG cell. Thus, while the term is typically used with respect to a gene, the term “overexpression” may also be used with respect to an encoded protein to refer to the increased level of the protein resulting from the overexpression of its encoding gene. The overexpression of a gene encoding a protein may be achieved by various methods known in the art, e.g., by increasing the number of copies of the gene that encodes the protein, or by increasing the binding strength of the promoter region or the ribosome binding site in such a way as to increase the transcription or the translation of the gene that encodes the protein.

The phrase “CAR/NR1I3 gene” means a nucleic acid that has a nucleic acid sequence at least 75, 80, 90, 95, 99, or 100% identical to the nucleic acid sequence of the naturally-occurring CAR/NR1I3 gene, (GenBank Accession No. NG_029113.1 and that has at least 50, 75, 80, 90, 95, or 100% of the CAR protein activity of a naturally-occurring human CAR protein assayed under identical conditions.

The overexpression of the CAR protein in said modified HepaRG cell may be accomplished by any means, including but not limited to stable transfection or transient transfection with a nucleic acid construct comprising the nucleic acid sequence of an exogenous copy of the CAR/NR1I3 gene.

As used herein the term “nucleic acid expression construct” refers to a nucleic acid construct which includes the nucleic acid encoding the CAR protein and at least one promoter for directing transcription of nucleic acid in a HepaRG cell. Further details of suitable transformation approaches are provided herein.

As used herein, the term “exogenous copy” of a gene refers to the non-genomic copy of a gene, and/or an added copy of gene that is introduced into the cell.

The expressions “transfection” or “transfected” refers to the introduction of a nucleic acid into a cell under conditions allowing expression of the protein. In general the nucleic acid is a DNA sequence, in particular a vector or a plasmid carrying a gene of interest under a suitable promoter, whose expression is controlled by said promoter. However, the term transfection also comprises RNA transfection.

The term “stable transfection”, “stably transfected” or “stable transfected” is here also used to refer to cells carrying in the genome of the HepaRG cell the nucleic acid construct comprising at least one more copy of the CAR gene compared to the unmodified HepaRG cell. In a preferred embodiment, a gene transfer system is used to transfect the HepaRG cell. One particularly gene transfer system applicable for “stably transfecting” cells is based on recombinant retroviruses. Since integration of the proviral DNA is an obligatory step during the retroviral replication cycle, infection of cells with a recombinant retrovirus will give rise to a very high proportion of cells that have integrated the gene of interest and are thus stably transfected.

As used herein the term “transient transfection” as used within this application refers to a process in which the nucleic acid introduced into a cell is not required to integrate into the genome or chromosomal DNA of that cell. It is in fact predominantly maintained as an extrachromosomal element, e.g. as an episome, in the cell. Transcription processes of the nucleic acid of the episome are not affected and e.g. a protein encoded by the nucleic acid of the episome is produced.

Methods of transient or stable transfection are well known in the art. The skilled artisan is familiar with the various transfection methods such those using carrier molecules like cationic lipids such as DOTAP (Roche), DOSPER (Roche), Fugene (Roche), Transfectam® (Promega), TransFast™ (Promega) and Tfx™ (Promega), Lipofectamine (Invitrogene) and 293Fectin™ (Invitrogene), or calcium phosphate and DEAE dextran. He is also familiar with brute-force transfection techniques. These include electroporation, bombardment with nucleic-acid-coated carrier particles (gene gun), and microinjection. Finally the skilled artisan is also familiar with nucleic acid transfection using viral vectors.

Overexpression of CAR in HepaRG cells results in surprising structural changes, including changes of levels of nucleic acids and proteins as are summarized in Table 1. The level of said overexpression must be at least higher than in unmodified HepaRG cells. Preferably at least 2, 3, 4, 5, 6, 7, 8, 9, 10, 15 or 20 times higher than in unmodified HepaRG cells.

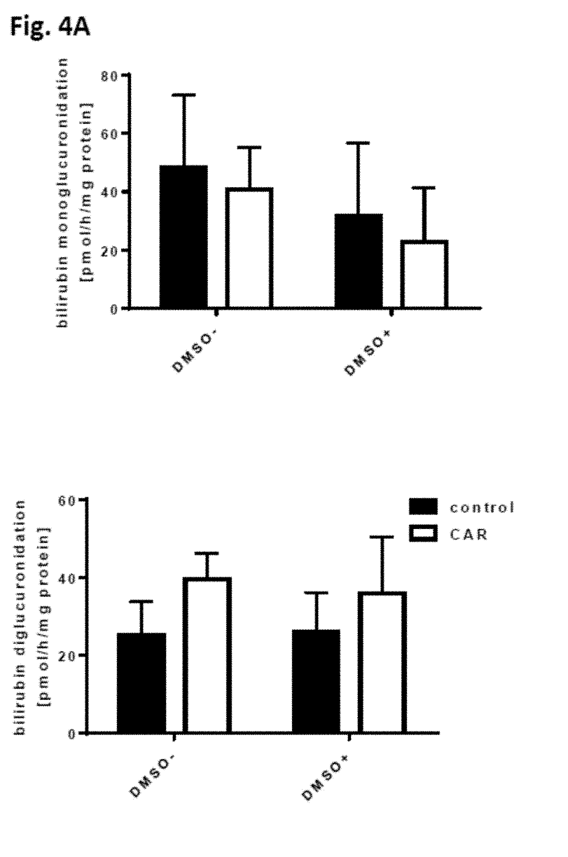

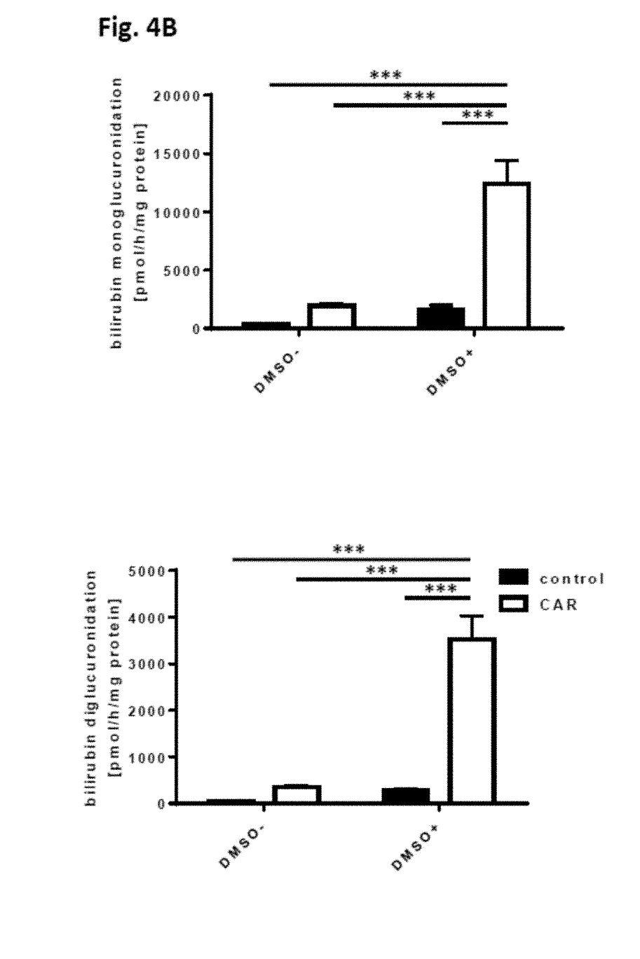

In a preferred embodiment, said overexpression is in an amount resulting in increased UGT1A1 transcript level when compared to unmodified HepaRG cells. Therefore, said level of UGT1A1 is preferably higher than 37.4%, or 102.3% of the expression level in unmodified HepaRG cells. Preferably, bilirubin mono- and di-glucuronidation levels are increased in modified HepaRG cells compared to controls. Preferably, by respectively at least 5.3× (for mono-glucuronidation levels) or at least 8.3× (for di-glucuronidation levels) in cells cultured without DMSO and by respectively at least 7.7× (for mono-glucuronidation levels) or at least 12.8× (for di-glucuronidation levels) in cells cultured with DMSO (see FIG. 4B). Said comparison is determined in cell homogenates of said unmodified HepaRG cells, to which a non-limiting amount of UDP-glucuronic acid (UDPGA) co-factor is added.

In another preferred embodiment, said overexpression is in an amount resulting in a higher TC50 value for acetaminophen and/or amiodarone compared to unmodified HepaRG cells.

In another preferred embodiment, said overexpression is in an amount resulting in a higher clearance of warfarin and/or prednisolone compared to unmodified HepaRG cells.

The inventors herein found that overexpression of CAR can compensate for the addition of DMSO with regard to the expression and activity of phase I drug metabolism. Furthermore, the results also indicate that overexpression of CAR in combination with the addition of DMSO induces an altered, and improved differentiation of HepaRG cells into a more liver-like phenotype. This phenotype may be achieved if a certain minimum expression level of the CAR protein is present in the modified HepaRG cells of the invention. Therefore, in a preferred embodiment, said overexpression is in an amount exceeding 100% of human control liver tissue, preferably 200%, as determined after culturing in HepaRG medium containing 1.7% DMSO. The term “human control liver tissue” as used herein refers to a healthy human liver biopsy, preferably the average of biopsies of at least 2 different donors.

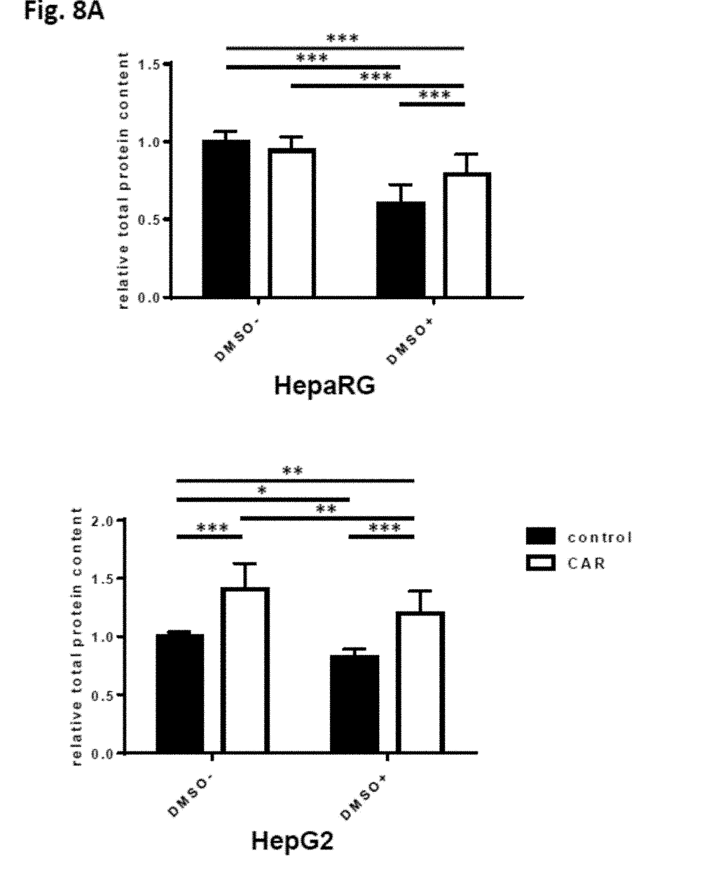

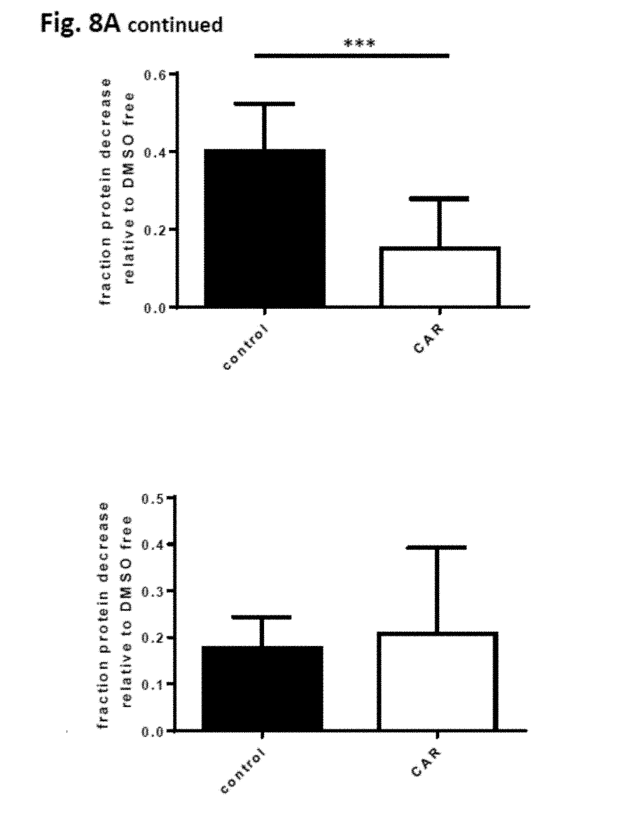

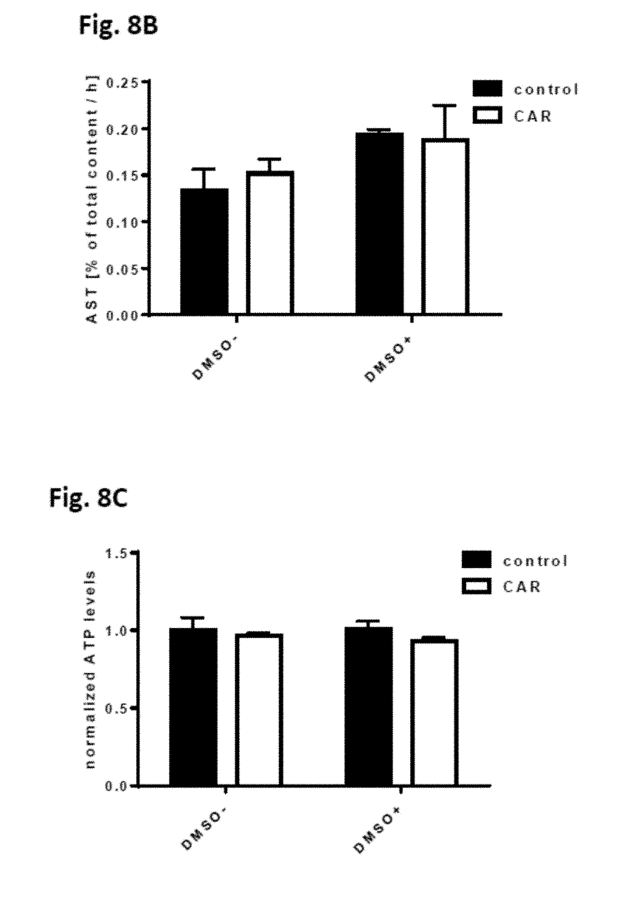

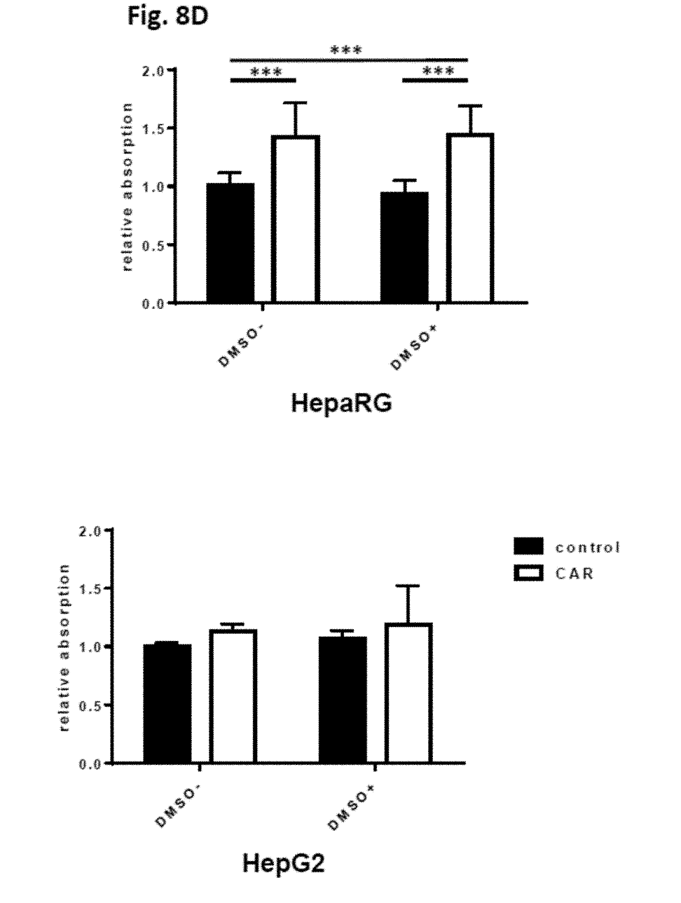

The modified HepaRG cells of the invention are less affected by DMSO treatment compared to unmodified HepaRG cells. Said modified HepaRG cells only showed a 19% reduction of total protein content when compared with untreated cells (see FIG. 8A). WST activity corrected for protein, which is a marker for cellular NADH levels is increased by CAR overexpression (see FIG. 8D). Therefore, it is preferred that said overexpression in modified HepaRG cells is in an amount which causes an increase in WST-1 activity by at least 30%, more preferably at least 40% in comparison to an unmodified HepaRG cell. The term “WST activity” as used herein refers to relative cellular NADH levels as assessed using a WST-1 assay. The WST-1 assay is based on the extracellular reduction of a tetrazolium dye via trans-membrane electron transport (Berridge, Herst et al. 2005). NADH is the electron donor and is mainly produced by the mitochondrial tricarboxylic acid cycle. The assay is performed by washing cells once with PBS and then incubating for 15 minutes in 20× diluted Cell Proliferation Reagent WST-1 (Roche) dissolved into phenol-red free HepaRG culture medium. Supernatants may then be transferred to a clear 96 well plate and absorbance at A=450 nm, subtracted by absorbance at A=620 nm, read on a plate reader (for instance the Synergy HT from BioTek). The WST-1 activity is preferably determined using the assay as described herein.

Preferably, said overexpression is stable for at least 7 passages.

The inventors observed increased P. falciparum infection in the modified HepaRG cells of the invention 3 days after infection when compared to normal HepaRG cells.

In an embodiment, the modified HepaRG cells of the invention are infected with malaria parasites. Before entering the erythrocyte stage of their life cycle Plasmodium parasites first enter a hepatic stage where they infect hepatocytes in order to mature and replicate (Prudencio, Rodriguez et al. 2006). During this liver stage, sporozoites in one hepatocyte can multiply to up to thousands of merozoites before being released back into the blood stream (Sturm, Amino et al. 2006). Interestingly, the inventors observed increased P. falciparum infection in the modified HepaRG cells 3 days after infection when compared to normal HepaRG cells. Therefore, said modified HepaRG cells infected with a malaria parasite provide an easy-to-infect model for studies on the liver stage of the malaria parasites. In a preferred embodiment, wherein said modified cell is infected by a malaria parasite. Said malaria parasite may be any Plasmodium parasite, e.g. P. falciparum and P. reichenowi. Preferably, said parasite is P. falciparum. Preferably, said malaria parasite is a sporozoite.

In a highly preferred embodiment, said modified HepaRG cell is as deposited on [Oct. 5, 2016] at the DSMZ, under No. DSM ACC3291.

Producing Modified HepaRG Cells

Suitably, the modified HepaRG cell according to the invention may be produced by a method comprising steps of: (a) providing a cell culture of HepaRG cells, (b) modifying the HepaRG cells using a nucleic acid construct comprising the CAR/NR1I3 gene and a selection marker, and (c) selecting a modified HepaRG cell using the selection marker.

In a preferred embodiment, transfection or transduction is performed using a selection marker to distinguish modified HepaRG cells wherein the nucleic acid construct is successfully integrated from unmodified HepaRG cells. Preferably, successfully transduced HepaRG cells are obtained by selection for puromycin resistance. Preferably, said nucleic acid construct comprises the phosphoglycerate kinase (Pgk-1) promoter driven puromycin N-acetyl-transferase gene. Preferably, a third generation lentiviral vector system (Dull, Zufferey et al. 1998, Zufferey, Dull et al. 1998) is used for transduction.

Transduction is preferably done using low passage HepaRG cells. Preferably HepaRG cells with a passage of 12 or lower are used. A preferred transduction method is using DEAE dextran. Preferably, transduction is performed using viral particles produced with a plasmid. Preferably, said plasmid comprises the nucleic acid sequence according to SEQ ID NO:1

SEQ ID NO: 1:

| | |
|---|---|
| ctagtccagt gtggtggaat tgcccttgac gtcatggcca | 60 |
| gtagggaaga tgagctgagg | |
| aactgtgtgg tatgtgggga ccaagccaca ggctaccact | 120 |
| ttaatgcgct gacttgtgag | |
| ggctgcaagg gtttcttcag gagaacagtc agcaaaagca | 180 |
| ttggtcccac ctgccccttt | |
| gctggaagct gtgaagtcag caagactcag aggcgccact | 240 |
| gcccagcctg caggttgcag | |
| aagtgcttag atgctggcat gaggaaagac atgatactgt | 300 |
| cggcagaagc cctggcattg | |
| cggcgagcaa agcaggccca gcggcgggca cagcaaacac | 360 |
| ctgtgcaact gagtaaggag | |
| caagaagagc tgatccggac actcctgggg gcccacaccc | 420 |
| gccacatggg caccatgttt | |
| gaacagtttg tgcagtttag gcctccagct catttgttca | 480 |
| tccatcacca gcccttgccc | |
| accctggccc ctgtgctgcc tctggtcaca cacttcgcag | 540 |
| acatcaacac tttcatggta | |
| ctgcaagtca tcaagtttac taaggacctg cctgtcttcc | 600 |
| gttccctgcc cattgaagac | |
| cagatctccc ttctcaaggg agcagctgtg gaaatctgtc | 660 |
| acatcgtact caataccact | |
| ttctgtctcc aaacacaaaa cttcctctgc gggcctcttc | 720 |
| gctacacaat tgaagatgga | |
| gcccgtgtgg ggttccaggt agagtttttg gagttgctct | 780 |
| ttcacttcca tggaacacta | |
| cgaaaactgc agctccaaga gcctgagtat gtgctcttgg | 840 |
| ctgccatggc cctcttctct | |
| cctgaccgac ctggagttac ccagagagat gagattgatc | 900 |
| agctgcaaga ggagatggca | |
| ctgactctgc aaagctacat caagggccag cagcgaaggc | 960 |
| cccgggatcg gtttctgtat | |
| gcgaagttgc taggcctgct ggctgagctc cggagcatta | 1020 |
| atgaggccta cgggtaccaa | |
| atccagcaca tccagggcct gtctgccatg atgccgctgc | 1080 |
| tccaggagat ctgcagctga | |
| ggccatgctc acttccttcc ccagctcacc tggaacaccc | 1140 |
| tggatacact ggagtgggaa | |
| aatgctggga ccaagggcaa ttctgcagat atccagcaca | 1200 |
| gtggcggccg ctcgagtcta | |
| gacatatggg taccatgcat gtattcaatc taagcaggct | 1260 |
| ttcactttct cgccaactta | |
| caaggccttt ctgtgtaaac aatacctgaa cctttacccc | 1320 |
| gttgcccggc aacggccacc | |
| tctgtgccaa gtgtttgctg acgcaacccc cactggctgg | 1380 |
| ggcttggtca tgggccatca | |
| gcgcatgcgt ggaacctttt cggctcctct gccgatccat | 1440 |
| actgcggaac tcctagccgc | |
| ttgttttgct cgcagcaggt ctggagcaaa cattatcggg | 1500 |
| actgataact ctgttgtcct | |
| atcccgcaaa tatacatcgt ttccatggct gctaggctgt | 1560 |
| gctgccaact ggatcctgcg | |
| cgggacgtcc tttgtttacg tcccgtcggc gctgaatcct | 1620 |
| gcggacgacc cttctcgggg | |
| tcgcttggga ctctctcgtc cccttctccg tctgccgttc | 1680 |
| cgaccgacca cggggcgcac | |
| ctctctttac gcggactccc cgtctgtgcc ttctcatctg | 1740 |
| ccggaccgtg tgcacttcgc | |
| ttcacctctg cacgtcgcat ggagaccacc gtgaacgccc | 1800 |
| accaaatatt gcccaaggtc | |
| ttacataaga ggactcttgg actctcagca atgtcaacga | 1860 |
| ccgaccttga ggcatacttc | |
| aaagactgtt tgtttaaaga ctgggaggag ttgggggagg | 1920 |
| agattaggtt aaaggtcttt | |
| gtactaggag gctgtaggca taaattggtc tgcgcaccag | 1980 |
| caccatgtat cactagagcg | |
| gccgccaccg cggaattccg tttaagacca atgacttaca | 2040 |
| aggcagctgt agatcttagc | |
| cactttttaa aagaaaaggg gggactggaa gggctaattc | 2100 |
| actcccaacg aagacaagat | |
| ctgctttttg cttgtactgg gtctctctgg ttagaccaga | 2160 |
| tctgagcctg ggagctctct | |
| ggctaactag ggaacccact gcttaagcct caataaagct | 2220 |
| tgccttgagt gcttcaagta | |
| gtgtgtgccc gtctgttgtg tgactctggt aactagagat | 2280 |
| ccctcagacc cttttagtca | |
| gtgtggaaaa tctctagcag tagtagttca tgtcatctta | 2340 |
| ttattcagta tttataactt | |
| gcaaagaaat gaatatcaga gagtgagagg aacttgttta | 2400 |
| ttgcagctta taatggttac | |
| aaataaagca atagcatcac aaatttcaca aataaagcat | 2460 |
| ttttttcact gcattctagt | |
| tgtggtttgt ccaaactcat caatgtatct tatcatgtct | 2520 |
| ggctctagct atcccgcccc | |
| taactccgcc cagttccgcc cattctccgc cccatggctg | 2580 |
| actaattttt tttatttatg | |
| cagaggccga ggccgcctcg gcctctgagc tattccagaa | 2640 |
| gtagtgagga ggcttttttg | |
| gaggcctagg cttttgcgtc gagacgtacc caattcgccc | 2700 |
| tatagtgagt cgtattacgc | |
| gcgctcactg gccgtcgttt tacaacgtcg tgactgggaa | 2760 |
| aaccctggcg ttacccaact | |
| taatcgcctt gcagcacatc cccctttcgc cagctggcgt | 2820 |
| aatagcgaag aggcccgcac | |
| cgatcgccct tcccaacagt tgcgcagcct gaatggcgaa | 2880 |
| tggcgcgacg cgccctgtag | |
| cggcgcatta agcgcggcgg gtgtggtggt tacgcgcagc | 2940 |
| gtgaccgcta cacttgccag | |
| cgccctagcg cccgctcctt tcgctttctt cccttccttt | 3000 |
| ctcgccacgt tcgccggctt | |
| tccccgtcaa gctctaaatc gggggctccc tttagggttc | 3060 |
| cgatttagtg ctttacggca | |
| cctcgacccc aaaaaacttg attagggtga tggttcacgt | 3120 |
| agtgggccat cgccctgata | |
| gacggttttt cgccctttga cgttggagtc cacgttcttt | 3180 |
| aatagtggac tcttgttcca | |
| aactggaaca acactcaacc ctatctcggt ctattctttt | 3240 |
| gatttataag ggattttgcc | |
| gatttcggcc tattggttaa aaaatgagct gatttaacaa | 3300 |
| aaatttaacg cgaattttaa | |
| caaaatatta acgtttacaa tttcccaggt ggcacttttc | 3360 |
| ggggaaatgt gcgcggaacc | |
| cctatttgtt tatttttcta aatacattca aatatgtatc | 3420 |
| cgctcatgag acaataaccc | |
| tgataaatgc ttcaataata ttgaaaaagg aagagtatga | 3480 |
| gtattcaaca tttccgtgtc | |
| gcccttattc ccttttttgc ggcattttgc cttcctgttt | 3540 |
| ttgctcaccc agaaacgctg | |
| gtgaaagtaa aagatgctga agatcagttg ggtgcacgag | 3600 |
| tgggttacat cgaactggat | |
| ctcaacagcg gtaagatcct tgagagtttt cgccccgaag | 3660 |
| aacgttttcc aatgatgagc | |
| acttttaaag ttctgctatg tggcgcggta ttatcccgta | 3720 |
| ttgacgccgg gcaagagcaa | |
| ctcggtcgcc gcatacacta ttctcagaat gacttggttg | 3780 |
| agtactcacc agtcacagaa | |
| aagcatctta cggatggcat gacagtaaga gaattatgca | 3840 |
| gtgctgccat aaccatgagt | |
| gataacactg cggccaactt acttctgaca acgatcggag | 3900 |
| gaccgaagga gctaaccgct | |
| tttttgcaca acatggggga tcatgtaact cgccttgatc | 3960 |
| gttgggaacc ggagctgaat | |
| gaagccatac caaacgacga gcgtgacacc acgatgcctg | 4020 |
| tagcaatggc aacaacgttg | |
| cgcaaactat taactggcga actacttact ctagcttccc | 4080 |
| ggcaacaatt aatagactgg | |
| atggaggcgg ataaagttgc aggaccactt ctgcgctcgg | 4140 |
| cccttccggc tggctggttt | |
| attgctgata aatctggagc cggtgagcgt gggtctcgcg | 4200 |
| gtatcattgc agcactgggg | |
| ccagatggta agccctcccg tatcgtagtt atctacacga | 4260 |
| cggggagtca ggcaactatg | |
| gatgaacgaa atagacagat cgctgagata ggtgcctcac | 4320 |
| tgattaagca ttggtaactg | |
| tcagaccaag tttactcata tatactttag attgatttaa | 4380 |
| aacttcattt ttaatttaaa | |
| aggatctagg tgaagatcct ttttgataat ctcatgacca | 4440 |
| aaatccctta acgtgagttt | |
| tcgttccact gagcgtcaga ccccgtagaa aagatcaaag | 4500 |
| gatcttcttg agatcctttt | |
| tttctgcgcg taatctgctg cttgcaaaca aaaaaaccac | 4560 |
| cgctaccagc ggtggtttgt | |
| ttgccggatc aagagctacc aactcttttt ccgaaggtaa | 4620 |
| ctggcttcag cagagcgcag | |
| ataccaaata ctgtccttct agtgtagccg tagttaggcc | 4680 |
| accacttcaa gaactctgta | |
| gcaccgccta catacctcgc tctgctaatc ctgttaccag | 4740 |
| tggctgctgc cagtggcgat | |
| aagtcgtgtc ttaccgggtt ggactcaaga cgatagttac | 4800 |
| cggataaggc gcagcggtcg | |
| ggctgaacgg ggggttcgtg cacacagccc agcttggagc | 4860 |
| gaacgaccta caccgaactg | |
| agatacctac agcgtgagct atgagaaagc gccacgcttc | 4920 |
| ccgaagggag aaaggcggac | |
| aggtatccgg taagcggcag ggtcggaaca ggagagcgca | 4980 |
| cgagggagct tccaggggga | |
| aacgcctggt atctttatag tcctgtcggg tttcgccacc | 5040 |
| tctgacttga gcgtcgattt | |
| ttgtgatgct cgtcaggggg gcggagccta tggaaaaacg | 5100 |
| ccagcaacgc ggccttttta | |
| cggttcctgg ccttttgctg gccttttgct cacatgttct | 5160 |
| ttcctgcgtt atcccctgat | |
| tctgtggata accgtattac cgcctttgag tgagctgata | 5220 |
| ccgctcgccg cagccgaacg | |
| accgagcgca gcgagtcagt gagcgaggaa gcggaagagc | 5280 |
| gcccaatacg caaaccgcct | |
| ctccccgcgc gttggccgat tcattaatgc agctggcacg | 5340 |
| acaggtttcc cgactggaaa | |
| gcgggcagtg agcgcaacgc aattaatgtg agttagctca | 5400 |
| ctcattaggc accccaggct | |
| ttacacttta tgcttccggc tcgtatgttg tgtggaattg | 5460 |
| tgagcggata acaatttcac | |
| acaggaaaca gctatgacca tgattacgcc aagcgcgcaa | 5520 |
| ttaaccctca ctaaagggaa | |
| caaaagctgg agctgcaagc ttaatgtagt cttatgcaat | 5580 |
| actcttgtag tcttgcaaca | |
| tggtaacgat gagttagcaa catgccttac aaggagagaa | 5640 |
| aaagcaccgt gcatgccgat | |
| tggtggaagt aaggtggtac gatcgtgcct tattaggaag | 5700 |
| gcaacagacg ggtctgacat | |
| ggattggacg aaccactgaa ttggaggcgt ggcctgggcg | 5760 |
| ggactgggga gtggcgagcc | |
| ctcagatcct gcatataagc agctgctttt tgcctgtact | 5820 |
| gggtctctct ggttagacca | |
| gatctgagcc tgggagctct ctggctaact agggaaccca | 5880 |
| ctgcttaagc ctcaataaag | |
| cttgccttga gtgcttcaag tagtgtgtgc ccgtctgttg | 5940 |
| tgtgactctg gtaactagag | |
| atccctcaga cccttttagt cagtgtggaa aatctctagc | 6000 |
| agtggcgccc gaacagggac | |
| ctgaaagcga aagggaaacc agagctctct cgacgcagga | 6060 |
| ctcggcttgc tgaagcgcgc | |
| acggcaagag gcgaggggcg gcgactggtg agtacgccaa | 6120 |
| aaattttgac tagcggaggc | |
| tagaaggaga gagatgggtg cgagagcgtc agtattaagc | 6180 |
| gggggagaat tagatcgcga | |
| tgggaaaaaa ttcggttaag gccaggggga aagaaaaaat | 6240 |
| ataaattaaa acatatagta | |
| tgggcaagca gggagctaga acgattcgca gttaatcctg | 6300 |
| gcctgttaga aacatcagaa | |
| ggctgtagac aaatactggg acagctacaa ccatcccttc | 6360 |
| agacaggatc agaagaactt | |
| agatcattat ataatacagt agcaaccctc tattgtgtgc | 6420 |
| atcaaaggat agagataaaa | |
| gacaccaagg aagctttaga caagatagag gaagagcaaa | 6480 |
| acaaaagtaa gaccaccgca | |
| cagcaagcgg ccgctgatct tcagacctgg aggaggagat | 6540 |
| atgagggaca attggagaag | |
| tgaattatat aaatataaag tagtaaaaat tgaaccatta | 6600 |
| ggagtagcac ccaccaaggc | |
| aaagagaaga gtggtgcaga gagaaaaaag agcagtggga | 6660 |
| ataggagctt tgttccttgg | |
| gttcttggga gcagcaggaa gcactatggg cgcagcctca | 6720 |
| atgacgctga cggtacaggc | |
| cagacaatta ttgtctggta tagtgcagca gcagaacaat | 6780 |
| ttgctgaggg ctattgaggc | |
| gcaacagcat ctgttgcaac tcacagtctg gggcatcaag | 6840 |
| cagctccagg caagaatcct | |
| ggctgtggaa agatacctaa aggatcaaca gctcctgggg | 6900 |
| atttggggtt gctctggaaa | |
| actcatttgc accactgctg tgccttggaa tgctagttgg | 6960 |
| agtaataaat ctctggaaca | |
| gattggaatc acacgacctg gatggagtgg gacagagaaa | 7020 |
| ttaacaatta cacaagctta | |
| atacactcct taattgaaga atcgcaaaac cagcaagaaa | 7080 |
| agaatgaaca agaattattg | |
| gaattagata aatgggcaag tttgtggaat tggtttaaca | 7140 |
| taacaaattg gctgtggtat | |
| ataaaattat tcataatgat agtaggaggc ttggtaggtt | 7200 |
| taagaatagt ttttgctgta | |
| ctttctatag tgaatagagt taggcaggga tattcaccat | 7260 |
| tatcgtttca gacccacctc | |
| ccaaccccga ggggacccga caggcccgaa ggaatagaag | 7320 |
| aagaaggtgg agagagagac | |
| agagacagat ccattcgatt agtgaacgga tctcgacggt | 7380 |
| atcgatcacg agactagcct | |
| cgagcggccg ccagtgtgat ggatcgatga attctaccgg | 7440 |
| gtaggggagg cgcttttccc | |
| aaggcagtct ggagcatgcg ctttagcagc cccgctgggc | 7500 |
| acttggcgct acacaagtgg | |
| cctctggcct cgcacacatt ccacatccac cggtaggcgc | 7560 |
| caaccggctc cgttctttgg | |
| tggccccttc gcgccacctt ctactcctcc cctagtcagg | 7620 |
| aagttccccc ccgccccgca | |
| gctcgcgtcg tgcaggacgt gacaaatgga agtagcacgt | 7680 |
| ctcactagtc tcgtgcagat | |
| ggacagcacc gctgagcaat ggaagcgggt aggcctttgg | 7740 |
| ggcagcggcc aatagcagct | |
| ttgctccttc gctttctggg ctcagaggct gggaaggggt | 7800 |
| gggtccgggg gcgggctcag | |
| gggcgggctc aggggcgggg cgggcgcccg aaggtcctcc | 7860 |
| ggaggcccgg cattctgcac | |
| gcttcaaaag cgcacgtctg ccgcgctgtt ctcctcttcc | 7920 |
| tcatctccgg gcctttcgac | |
| cgatccagcc gccaccatga ccgagtacaa gcccacggtg | 7980 |
| cgcctcgcca cccgcgacga | |
| cgtcccccgg gccgtacgca ccctcgccgc cgcgttcgcc | 8040 |
| gactaccccg ccacgcgcca | |
| caccgtcgac ccggaccgcc acatcgagcg ggtcaccgag | 8100 |
| ctgcaagaac tcttcctcac | |
| gcgcgtcggg ctcgacatcg gcaaggtgtg ggtcgcggac | 8160 |
| gacggcgccg cggtggcggt | |
| ctggaccacg ccggagagcg tcgaagcggg ggcggtgttc | 8220 |
| gccgagatcg gcccgcgcat | |
| ggccgagttg agcggttccc ggctggccgc gcagcaacag | 8280 |
| atggaaggcc tcctggcgcc | |
| gcaccggccc aaggagcccg cgtggttcct ggccaccgtc | 8340 |
| ggcgtctcgc ccgaccacca | |
| gggcaagggt ctgggcagcg ccgtcgtgct ccccggagtg | 8400 |
| gaggcggccg agcgcgccgg | |
| ggtgcccgcc ttcctggaga cctccgcgcc ccgcaacctc | 8460 |
| cccttctacg agcggctcgg | |
| cttcaccgtc accgccgacg tcgaggtgcc cgaaggaccg | 8520 |
| cgcacctggt gcatgacccg | |
| caagcccggt gcctgacgcc cgccccacga cccgcagcgc | 8580 |
| ccgaccgaaa ggagcgcacg | |
| accccatggc tccgaccgaa gccacccggg gcggccccgc | 8640 |
| cgaccccgca cccgcccccg | |
| aggcccaccg cgggggacac accgaacacg ccgaccctgc | 8700 |
| tgaacacgcg gcgcagttcg | |
| gtgcccagga gcggatcgaa attgatgatc tattaaacaa | 8760 |
| taaagatgtc cactaaaatg | |
| gaagtttttc ctgtcatact ttgttaagaa gggtgagaac | 8820 |
| agagtaccta cattttgaat | |
| ggaaggattg gagctacggg ggtgggggtg gggtgggatt | 8880 |
| agataaatgc ctgctcttta | |
| ctgaaggctc tttactattg ctttatgata atgtttcata | 8940 |
| gttggatatc ataatttaaa | |
| caagcaaaac caaattaagg gccagctcat tcctcccact | 9000 |
| catgatctat agatctatag | |
| atctctcgtg ggatcattgt ttttctcttg attcccactt | 9060 |
| tgtggttcta agtactgtgg | |
| tttccaaatg tgtcagtttc atagcctgaa gaacgagatc | 9120 |
| agcagcctct gttccacata | |
| cacttcattc tcagtattgt tttgccaagt tctaattcca | 9180 |
| tcagaagctt cagctgctcg | |
| aatctgcaga attcgccctt cagtatcgat aagcttacaa | 9240 |
| atggcagtat tcatccacaa | |
| ttttaaaaga aaagggggga ttggggggta cagtgcaggg | 9300 |
| gaaagaatag tagacataat | |
| agcaacagac atacaaacta aagaattaca aaaacaaatt | 9360 |
| acaaaaattc aaaattttcg | |
| ggtttattac agggacagca gagatccact ttggaatcga | 9420 |
| taaggaaggg cgaattccag | |
| cacactggcg gccgttacta gatcgaattc ccacggggtt | 9480 |
| ggggttgcgc cttttccaag | |
| gcagccctgg gtttgcgcag ggacgcggct gctctgggcg | 9540 |
| tggttccggg aaacgcagcg | |
| gcgccgaccc tgggtctcgc acattcttca cgtccgttcg | 9600 |
| cagcgtcacc cggatcttcg | |
| ccgctaccct tgtgggcccc ccggcgacgc ttcctgctcc | 9660 |
| gcccctaagt cgggaaggtt | |
| ccttgcggtt cgcggcgtgc cggacgtgac aaacggaagc | 9720 |
| cgcacgtctc actagtaccc | |
| tcgcagacgg acagcgccag ggagcaatgg cagcgcgccg | 9780 |
| accgcgatgg gctgtggcca | |
| atagcggctg ctcagcgggg cgcgccgaga gcagcggccg | 9840 |
| ggaaggggcg gtgcgggagg | |
| cggggtgtgg ggcggtagtg tgggccctgt tcctgcccgc | 9900 |
| gcggtgttcc gcattctgca | |
| agcctccgga gcgcacgtcg gcagtcggct ccctcgttga | 9960 |
| ccgaatcacc gacctctctc | |
| cccaggggga tccaccggtt gatcagtcga cgttaacgct | 10004 |
| agct | |

Culturing

The invention provides a method of culturing the modified HepaRG cell as defined above in a culture medium. As used herein the term ‘cell culturing’ refers to cells growing in suspension or adherent, in roller bottles, flasks, glass or stainless steel cultivations vessels, and the like. Large scale approaches, such as bioreactors, are also encompassed by the term ‘cell culturing’. Cell culture procedures for both large and small-scale production of polypeptides are encompassed by the present invention. Procedures including, but not limited to, a fluidized bed bioreactor, shaker flask culture, disposable bioreactor or stirred tank bioreactor system can be used and operated alternatively in a batch, split-batch, fed-batch, or perfusion mode.

The term “culturing” preferably refers to the maintenance of cells/cell lines in vitro in containers with medium supporting their proliferation and gene expression. Thus the culturing causes accumulation of the expressed secretable proteins in the culture medium. The medium normally contains supplements stabilizing the pH, as well as amino acids, lipids, trace elements, vitamins and other growth enhancing components. Culturing may be done in any suitable medium, for instance DMEM and HepaRG medium.

In a preferred embodiment, the method of culturing is performed in a three-dimensional cell culture system.

As used herein, “three-dimensional cell culture” refers to an artificially-created environment in which biological cells are permitted to grow or interact with their surroundings in all three dimensions. Preferred three-dimensional cell cultures may be based on scaffold techniques or scaffold-free techniques. Scaffold techniques include the use of solid scaffolds, hydrogels and other materials. Scaffold free techniques may employ another approach independent from the use scaffold. Scaffold-free methods include e.g. the use of low adhesion plates, hanging drop plates, micropatterned surfaces, and rotating bioreactors, magnetic levitation, and magnetic 3D bioprinting. In a preferred embodiment, said modified HepaRG are cultured in a bio-artificial liver in a dish (BALIAD). This BALIAD culture platform is based on the AMC bio-artificial liver (BAL), which is a perfused oxygenated bioreactor containing a non-woven polyester/cellulose matrix (Flendrig, la Soe et al. 1997). Cells are tightly attached to this matrix and grow in a three-dimensional configuration. The functionality of HepaRG cells is markedly improved by BAL culturing compared to monolayer culturing (Nibourg, Hoekstra et al. 2013). In a highly preferred embodiment, a high-throughput version of the BAL termed the BAL-in-a-dish (BALIAD) is used.

In a preferred embodiment, said culturing method comprises culturing in HepaRG culturing medium.

In an embodiment, the modified HepaRG cells of the invention are cultured in a medium substantially free of DMSO. As used herein, the term “substantially free of DMSO” means DMSO in an amount less than 0.2 w/w %.

The inventors found that culturing the modified HepaRG cells of the invention without DMSO resulted in activities of UGT1A1 and many CYPs that equaled or surpassed those of unmodified HepaRG cells cultured with DMSO and is therefore an excellent for, inter alia, studies on both synthetic and drug metabolic functions in hepatocytes. The additional increase in expression and activities of phase 1 and phase 2 drug metabolic enzymes in DMSO cultured modified HepaRG cells could enable more sensitive studies on low clearance compounds, (rare) metabolite formation, and detoxification mechanisms in HepaRG cells without the need to exogenously overexpress multiple drug metabolic enzymes. In addition, the modified HepaRG cells of the invention can be cultured in serum free culture medium, without the requirement of additional compensatory growth factors. Such cultures are highly preferably for use in the production of a protein produced by said cells, for example for the production of blood proteins.

In an embodiment, the modified HepaRG cells of the invention are cultured substantially without serum. An advantage thereof is that this makes the modified HepaRG cells more suitable for use as long-term serum-free producers of hepatocyte-derived blood proteins. The “serum-free”, “serum-free transfection” or “serum-free cultivation” refers to the transfection and culturing of cells in medium containing suitable supplements except any kind of serum or compensatory growth factors. Supplements are selected from amino acids, lipids, trace elements, vitamins and other growth enhancing components, as insulin and corticosteroids. Often the “serum-free” culture conditions are even more stringent and, if no exogenous protein is added, or already included in the medium, the medium is called “protein-free”.

The term “substantially serum free” or “substantially without serum” as used herein means that whole serum is absent, and the medium has no serum constituents or a minimal number of constituents from serum or other sources. Preferably, the culturing without serum is maintained for at least 3, 4, 5, 6, 7, 8, 9, 10, 11, 12, 13, or 14 days.

The invention further provides a modified HepaRG cell obtainable by any of the methods as defined above.

Cell Culture Comprising Modified HepaRG Cells

The invention further provides compositions comprising any of the modified HepaRG cells as defined herein. In a preferred embodiment, said composition comprises a suitable culture medium. As used herein the terms ‘cell culture medium’ and ‘culture medium’ as used interchangeably within the current invention refer to a nutrient solution used for growing mammalian cells. Such a nutrient solution generally includes various factors necessary for growth and maintenance of the cellular environment. For example, a typical nutrient solution can include a basal media formulation, various supplements depending on the cultivation type and, occasionally, selection agents.

In a further preferred, said culture is a 3D culture. Preferably, said culture comprises a non-woven polyester/cellulose matrix. Preferably, said cell culture is a BALIAD cell culture.

DMSO culture of modified HepaRG cells induces the formation of a large subset of a morphologically distinct undifferentiated hepatocyte-like cell type, while the proportion of cholangiocyte-like cells is diminished greatly. Culture of HepaRG-CAR without DMSO results in increased drug metabolic capacity beyond that of normal HepaRG cells cultured with DMSO while maintaining the improved hepatic synthetic capabilities associated with DMSO free culture of HepaRG cells. Therefore, the cell culture is preferably characterized by the presence of hepatocyte-like cells in between the hepatocyte islands of more than 25%, preferably more than 30, 35, 40, 45, 50% of the total cells in culture. As used herein, the term “hepatocyte-like cell” refers to a cell that exhibits major characteristics of a hepatocyte such as glycogen synthesis, albumin, urea and bile syntheses. These hepatocyte-like cells, as well as the hepatocyte islands, can be detected by immunostaining for albumin.

In another preferred embodiment, said culture comprises DMSO. The inventors found that in DMSO cultured modified HepaRG cells exhibit a robust increase in clearance of two low turnover compounds: prednisolone and warfarin compared to unmodified HepaRG cells cultured with or without DMSO, which indicates that modified HepaRG cells could be of use as a predictive model for the clearance of slow metabolizable compounds, especially when they are metabolized by one or more CAR-target enzymes. In vitro prediction of clearance and metabolites of small molecules that are slowly metabolized remains a challenge (reviewed in (Hutzler, Ring et al. 2015), despite some promising developments like the relay method, and the Hepatopac and Hμrel hepatocyte co-cultures (Chan, Yu et al. 2013, Di, Atkinson et al. 2013, Bonn, Svanberg et al. 2016). The use of unmodified HepaRG cells often results in under prediction of the in vivo clearance of several low clearance compounds (Bonn, Svanberg et al. 2016). In a preferred embodiment, said cell culture comprises at least 0.5, 0.6, 0.7, 0.8, 0.9, 1.0, 1.1, 1.2, 1.3, 1.4, 1.5, 1.6 or 1.7 w/w % DMSO.

Uses of the Modified HepaRG Cells

The invention further provides a method of producing a protein of interest comprising the steps of: (a) providing a cell culture of modified HepaRG cells, (b) allow the expression of the protein of interest, and (c) isolate the protein of interest.

As used herein, the term “protein of interest” refers to a protein or a polypeptide that is produced by a host cell. Protein of interest is generally a protein that is commercially significant. The protein of interest may be either homologous or heterologous to the host cell.

Preferably, said step b. is performed in the absence of serum.

The invention further provides the use of the modified HepaRG cell according to the invention or the cell culture according to the invention in a method of determining clearance of a compound.

The inventors showed that in monolayer, modified HepaRG cells have an increased capacity for cleavage of the tetrazolium dye WST-1 into formazan compared to unmodified HepaRG cells, indicating higher levels of intracellular NAD(P)H. Since most ATP/NADH in the cell is generated in the tricarboxylic acid cycle cycle, this suggests that modified HepaRG cells have a higher basal mitochondrial activity. In addition, the inventors observed reduced lactate production and higher oxygen consumption in monolayer cultured modified HepaRG cells. When cultured in BALIADs, modified HepaRG cells showed increased oxygen consumption and even a switch from lactate production to lactate consumption, and a trend towards decreased glucose consumption compared to unmodified HepaRG cells. Moreover, the inventors presented an increased mitochondrial vs nuclear DNA ratio in modified HepaRG cells cultured in presence of DMSO compared to unmodified monolayer cultures matured in absence or presence of DMSO, which is an indicator of mitochondrial biogenesis. BALIAD culturing further stimulated the increased mitochondrial vs nuclear DNA ratio, compared to monolayer cultures to similar extent for unmodified and modified HepaRG cells. Taken together, these observations indicate that CAR overexpression induces a shift in the energy metabolism of particularly HepaRG cells leading to increased mitochondrial oxidative phosphorylation, particularly when cultured in BALIADs. In HepG2 cells this shift in metabolism was not observed, except for an increased mtDNA vs nuclear DNA ratio in DMSO treated cells. This shift in metabolism makes the modified HepaRG cells more sensitive as a model for studies targeted to mitochondria, including studies on mitochondrial toxicity of compounds.

Culture with DMSO reduces the synthetic capacity of HepaRG cells, with or without overexpression of CAR (this paper and (Hoekstra, Nibourg et al. 2011)). However, modified HepaRG cells cultured without DMSO resulted in activities of UGT1A1 and many CYPs that equaled or surpassed those of HepaRG cells cultured with DMSO and therefore may be a promising model for studies on both synthetic and drug metabolic functions in hepatocytes. The additional increase in expression and activities of phase 1 and phase 2 drug metabolic enzymes in DMSO cultured modified HepaRG cells could enable more sensitive studies on low clearance compounds, (rare) metabolite formation, and detoxification mechanisms in HepaRG cells without the need to exogenously overexpress multiple drug metabolic enzymes. Taken together, overexpression of CAR in HepaRG cells provides a model for further elucidating the role of CAR and its target genes in hepatic differentiation and detoxification of endogenous and exogenous compounds.

Modified HepaRG cells are less sensitive to drug-induced toxicity of amiodarone and acetaminophen, which may be the result of increased phase 1 and 2 drug metabolic enzyme activities. This effect makes the modified HepaRG cell of the invention useful to study the role of CAR during hepatotoxicity, for example in combination with HepaRG-CAR Knock Out cells. Therefore, the invention further provides a kit comprising a modified HepaRG cell of the invention and a HepaRG cell which lacks the CAR gene or wherein the endogenous CAR gene is underexpressed. The term “endogenous” use herein means the original copy of the gene found in the genome of the cell.

Modified HepaRG cells of the invention are more suitable for bioartificial liver application (BAL) than the unmodified HepaRG cells. Bioartificial livers are based on bioreactors with liver cells for the extracorporeal treatment of end-stage liver disease patients to bridge to liver transplantation or liver regeneration (Van Wenum et al., 2015). The added value of the modification is: 1. the enhanced decrease of toxins (e.g. neurotoxins) accumulating in the plasma during liver failure, 2. the improved elimination of lactate (accumulating in plasma during liver failure, leading to acidosis), 3. the increased ATP supply, which is required for energy-consuming processes, as protein synthesis, leading to correction of blood composition during liver failure and 4. The increased resistance to FBS depletion, which will reduce the high costs and long delays (6 months) associated with testing and selecting an FBS batch for HepaRG cells. Furthermore the risk of contamination with FBS-derived microorganisms and prion agents is reduced, leading to higher safety of the cell line. Moreover, the stability of the cell culture will be improved by decreasing FBS levels in the medium. The invention therefore provides modified HepaRG cells of the invention for use in the treatment of a subject. Preferably, said treatment is a treatment of a liver disease. The invention further provides the use of the modified HepaRG cells in a bioartificial liver. The invention further provides a bioartificial liver comprising the modified HepaRG cell of the invention.

Modified HepaRG cells of the invention are particularly more suitable for bioartificial liver application and other applications, since the cells are more resistant to a preservation period at 4° C. for 24 hours, which may be extended for longer period. In particular when the cells are treated with 5 mM N-acetylcysteine (NAC)+100 μM dopamine (DA), the phenotype of the cells is preserved. This enables the transport of a bioartificial liver with mature cells from the production facility to the end-user, with maintenance of functional output. Modified HepaRG cells of the invention are more stable during expansion of the cell mass. At each passage the cells are expanded to a 5-fold large culture surface. The unmodified HepaRG cells start to transform at Passage 20 after the isolation from the hepatocellular carcinoma, resulting in, amongst others, increased cell quantity, measured by protein content, loss of the structure in the monolayer culture with hepatocyte islands, loss of ammonia elimination and increased lactate production. The modified HepaRG cells show stability of phenotype until at least passage 33, which may even be further extended. The increased stability may be the result of an observed 2-fold lower production of mitochondrial superoxide in the modified HepaRG cells. Reactive oxygen species, among which mitochondrial superoxide are known to stimulate degenerative processes (Forkink, Smeikink et al., 2010).

In an embodiment, the invention provides the use of the modified HepaRG cell of the invention in a method to study infectious diseases, including but not limited to Hepatitis A, B, C, D and E. The modified HepaRG cells of the invention are very suitable for such application, because of their high level of differentiation. The invention further provides the modified HepaRG cell of the invention infected with a pathogen, preferably a virus, a parasite, a prion or a bacterium. Preferably, said virus comprises Hepatitis A, B, C, D or E.

The above disclosure generally describes the present invention. Unless defined otherwise, all technical and scientific terms used herein have the same meaning as those commonly understood by one of ordinary skill in the art to which this invention belongs. A more complete understanding can be obtained by reference to the following specific examples which are provided herein for purposes of illustration only, and are not intended to limit the scope of the invention.

### Example

Methods

Chemicals, Drugs, Antibodies

Primary antibodies: mouse monoclonal anti multidrug resistance-associated protein 2 (MRP2) (M₂III6) (Paulusma, Bosma et al. 1996), goat polyclonal anti-human albumin (A80-229A, Bethyl Laboratories). Secondary antibodies: goat polyclonal anti-mouse IgG Alexa Fluor 488 (A-11001, Thermo Fisher), donkey polyclonal anti-goat IgG Alexa Fluor 488 (A-11055, Thermo Fisher). Induction drugs: omeprazole (Cayman Chemical), CITCO (Santa Cruz Biotechnology), rifampicin (Sigma-Aldrich). Metabolism drugs: dextromethorphan hydrobromide monohydrate (Santa Cruz Biotechnology), bupropion hydrochloride (Cayman Chemical), chlorzoxazone (Sigma-Aldrich), testosterone (Sigma-Aldrich), tolbutamide (Sigma-Aldrich). Clearance drugs: warfarin (Sigma-Aldrich), theophylline anhydrous (Sigma-Aldrich), prednisolone (Sigma-Aldrich). All other, non-specified chemicals and reagents were purchased from Sigma-Aldrich.

Cell Culture

All cultures were kept at 37° C. in a humidified 5% CO₂ atmosphere.

The cell line HepaRG (Biopredic International, Rennes, France (Gripon, Rumin et al. 2002)) was cultured in William's E medium (Lonza) supplemented with 10% fetal bovine serum (Lonza), 100 U/ml penicillin (Lonza), 100 μg/ml streptomycin (Lonza), 2 mM L-glutamine (Lonza), 50 μM hydrocortisone hemisuccinate and 5 μg/ml insulin. HepaRG cells were maintained in T75 flasks during 2 weeks after which they were propagated in new flasks. Propagation was done by washing the cells twice with phosphate buffered saline (PBS) and incubating them with a mixture of Accutase (Innovative Cell Technologies), Accumax (Innovative Cell Technologies) and PBS (2:1:1) at 37° C. until detachment. The cells were then centrifuged for 5 minutes at 50 g and seeded at a 1:5 split ratio in new T75 flasks. For testing, the cells were fully matured during 28 days in 12- or 24-well culture plates. These cells were cultured for 14 days in normal HepaRG medium after which they were either switched to HepaRG medium containing 1.7% DMSO or maintained on normal HepaRG medium for an additional 14 days as indicated in the results. For testing the effect of CAR overexpression HepaRG and HepaRG-CAR cultures of similar passage (±1 passage) were compared, between passage 15-19.

To assess the effect of serum depletion mature 28 day HepaRG cell cultures were maintained in HepaRG medium without FBS either with or without 1.7% DMSO for 14 additional days.

Human embryonic kidney (HEK) 293T cells and human hepatoma HepG2 cells were obtained from ATCC and cultured in DMEM (Lonza) supplemented with 10% FBS, 100 U/ml penicillin, 100 μg/ml streptomycin and 2 mM L-glutamine.

HepG2 cells were maintained in T75 flasks until 80-90% confluency after which they were propagated in new flasks. The cells were propagated similarly as HepaRG cells. HepG2 cells were seeded at a 1:3-1:4 ratio in 12 or 24 well plates. Cells were cultured for 1 day in DMEM medium after which they were either switched to DMEM containing 1.7% DMSO or maintained on normal DMEM medium for 2-5 days as indicated in the results. HepG2 cells overexpressing complement factor 6 (C6) were a gift from Dr K. Fluiter, AMC, Amsterdam.

To assess the potential of C6 and fibrinogen production HepG2 and HepaRG cells were cultured for three days without FBS.

Bio-Artificial Liver in a Dish (BALIAD) Culture

1.8×10⁵ HepaRG cells were seeded on a sterile non-woven fibrous BAL matrix (DuPont™ Spunlaced Nonwoven Fabric-matrix (DuPont, Wilmington, Del., USA) disc (6 mm diameter, 0.35 mm thickness) in 100 μl of HepaRG medium in 96 well plate. After 3 hours the matrices were transferred to 500 μl HepaRG medium in 24 well plate. One to two weeks later the matrices were transferred to 1 mL HepaRG medium in 12 well plates in a New Brunswick S41i (Eppendorf) incubator with a rotating platform set at 60 rpm. Four weeks after seeding the matrices were used for experiments in new 12 well plates.

Construction of HepaRG-CAR and HepG2-CAR

Plasmid Construction

A murine phosphoglycerate kinase (Pgk-1) promoter driven puromycin N-acetyl-transferase gene (from plasmid pHA263Pur/PGKpur, a gift from Dr C. Paulusma, Academic Medical Center, Amsterdam, The Netherlands (Robanus-Maandag, Dekker et al. 1998)) was cloned into the PPTPGKPRE backbone plasmid (pRRLcpptPGKmcsPRESsin, a gift from Dr J. Seppen, Academic Medical Center, Amsterdam, The Netherlands (Seppen, Rijnberg et al. 2002)), yielding the plasmid pBAL117. In detail, pHA263Pur was digested with ClaI and XhoI, Klenow blunted, and the 1.8 kb fragment was subcloned into an EcoRV digest of PPTPGKPRE to generate pBAL117 and was verified by sequencing (BigDye Terminator, Thermo Fisher).

The NR1I3 (CAR) gene, (isoform 3) was cloned into the pBAL117 plasmid yielding the plasmid pBAL117xCAR. In detail, the plasmid pEF-hCAR (a gift from Prof Dr R. Kim, Vanderbilt University School of Medicine, Nashville, Tenn., USA (Tirona, Lee et al. 2003)) was digested with SpeI and XbaI and the 1 kb fragment was subcloned into a XbaI digest of pBAL117 to generate pBAL117xCAR, which was verified by sequencing.

Lentiviral Vector Production

HEK 293T cells were transiently transfected with pBAL117xCAR using polyethylenimine and a third generation lentiviral vector system (Dull, Zufferey et al. 1998, Zufferey, Dull et al. 1998). Four hours after transfection, the DMEM culture medium was refreshed. Medium containing viral particles, i.e. the viral DMEM, was harvested 44 hours following transfection, filtered through 0.45 μm filters (Millipore), and stored at −80° C.

Transduction

Twenty-four hours following seeding low passage (P12) HepaRG cells were transduced for 8 hours in 10 μg/ml diethylaminoethyl-dextran containing a 1:1 mixture of viral DMEM:HepaRG medium. The polyclonal and stable CAR overexpressing HepaRG line was obtained by selection for puromycin resistance during 8 days with 2.5 μg/ml puromycin starting from 1 day after transduction. HepG2-CAR was generated similarly, with the following alterations: DMEM was used during transduction and puromycin selection was done for 10 days at a concentration of 2 μg/ml.

The CAR-overexpressing HepaRG and HepG2 cell lines were cultured as described for their parental cell lines.

Lactate, Glucose and Ammonia Metabolism, Albumin Production, Total Protein and Cell Leakage

The cultures were washed twice with PBS and then exposed to phenol-red free HepaRG culture medium with 1.5 mM ¹⁵NH₄Cl (Sigma), 2.27 mM D-galactose (Sigma), and 2 mM L-lactate (Sigma) without DMSO. Medium samples were taken after 45 min, 8 and 24 hours of incubation. Production or elimination rates were established by calculating the concentration changes of different compounds, as indicated below, in the test media in time, and were corrected for protein content per well.

Protein levels of cell cultures were assessed with the Bio-Rad Protein Assay (Bio-Rad, Hercules, Calif.) according to manufacturer's instructions after lysis in 0.2 M NaOH for 1 h at 37° C. Ammonia concentrations in the test media were determined with the Ammonia (Rapid) kit (Megazyme, Bray, Ireland) according to manufacturer's instructions. Albumin protein levels in the test media were assessed with the human serum albumin duoset enzyme linked immunosorbent assay (ELISA) (R&D systems, Minneapolis, Minn.) according to the manufacturer's instructions. Lactate concentrations in test media were determined using the L-Lactate Acid kit (Megazyme) according to manufacturer's instructions. Glucose concentrations were determined using the Contour XT blood glucose meter (Bayer). Cell leakage was established by spectrophotometrical determination of aspartate aminotransferase (AST) levels of test medium samples and diluted cell lysates using a P800 Roche Diagnostics analyzer (Roche, Basel, Switzerland). AST levels are expressed relative to total cellular AST content (Nibourg, Huisman et al. 2010).

WST-1 Assay

Relative cellular NADH levels were assessed with the WST-1 assay. The WST-1 assay is based on the extracellular reduction of a tetrazolium dye via trans-membrane electron transport (Berridge, Herst et al. 2005). NADH is the electron donor and is mainly produced by the mitochondrial tricarboxylic acid cycle. The assay was performed by washing cells once with PBS and then adding 20× diluted Cell Proliferation Reagent WST-1 (Roche) dissolved into phenol-red free HepaRG culture medium for 15 minutes. Supernatants were transferred to a clear 96 well plate and absorbance at A=450 nm, subtracted by absorbance at A=620 nm, was read on a Synergy HT (BioTek) plate reader.

Oxygen Consumption

Oxygen consumption of fully differentiated HepaRG and HepaR-CAR monolayer cultures in 96-well Seahorse microplates was measured using the Seahorse XF96 (Seahorse Bioscience).

Oxygen consumption in BALIADs was fluorescently measured in 96 well round bottom OxoPlates (PreSens) (John, Klimant et al. 2003) on a Synergy HT (BioTek) plate reader. Oxoplates contain two fluorescent dyes at the bottom of the well: an oxygen-sensitive dye and a reference dye. Oxygen-insensitive reference fluorescence was measured with λ_{exc}=540±15 nm and λₑₘ=575±15 nm, while oxygen-sensitive fluorescence was measured with λ_{exc}=540±15 nm and λₑₘ=671±20 nm. BALIADs were transferred to OxoPlates into oxygen saturated culture medium and immediately measured every minute during 30 minutes in FBS- and phenol-red free HepaRG medium. As an indication for maximal physiological oxygen consumption the largest change in fluorescence over 5-6 minutes (i.e. steepest slope) was selected. The oxygen consumption of all cultures was normalized for total protein, measured as indicated above.

Measurement Mitochondrial Superoxide

MitoSOX™ Red reagent (Thermo Fisher) was used to measure superoxide. MitoSOX™ permeates live cells where it selectively targets mitochondria and is rapidly oxidized by superoxide, resulting in a fluorescent signal. Fluorescence was measured by fluorescence-activate cell sorting according to the manufacturer's instructions.

Immunofluorescence

Cells were washed 3× with cold PBS after they were fixed with 10% formalin (VWR) for 1 h at 4° C. The cells were permeabilized with 0.3% Triton-X 100 (Bio-Rad) at 4° C. for 15 minutes. Cells were then blocked with 10% FBS in PBS on ice for 1 h and incubated with primary antibody diluted in PBS at 4° C. overnight. The cells were washed 3× with cold PBS, incubated 2 h at 4° C. with secondary antibody Alexa Fluor 488 (Thermo Fisher) diluted 1:1000 in PBS and washed again 3× with cold PBS before incubation with DAPI-containing Vectashield (Vector Laboratories).

SDS PAGE and Western Blotting

Non-reduced culture medium samples (5 μl of 1000 μl total culture medium per sample) were diluted 1:1 in 2× Laemmli buffer (without β-mercaptoethanol or boiling), separated on a 8% poly-acrylamide gel and transferred to a Protran BA83/3 mm nitrocellulose membrane (GE Healthcare). Reduced culture medium samples were diluted 1:1 with 2× Laemmli buffer containing 5% β-mercaptoethanol and boiled for 5 minutes at 95° C. Membranes were blocked overnight at 4° C. in block buffer (5% milk powder in PBS), incubated for 1 hour at room temperature in block buffer with 1 μg/ml monoclonal rat anti-human C6 (7E5, Regenesance) or 1:1000 diluted polyclonal rabbit anti-human fibrinogen-HRP (P0445, Dako). Membranes incubated with anti-human C6 were washed 3× in wash buffer (PBS+0.05% Tween 20) and incubated for 1 hour at room temperature in block buffer with 1:1000 diluted rabbit anti-rat IgG-HRP (Dako). Membranes incubated with HRP-conjugated antibodies were washed 3× in wash buffer, developed with Lumi-Light PLUS (Roche), and detected using an LAS-3000 Imager (Fujifilm).

Transcript Levels

Total RNA was isolated using RNeasy kit (Qiagen) according to manufacturer's instructions. cDNA was synthesized from 1 μg RNA using a mix of 18S rRNA and gene-specific reverse transcriptase (RT)-primers and SuperScript III reverse transcriptase (Invitrogen) as described (Hoekstra, Deurholt et al. 2005), with the modification of using gene-specific anti-sense primers for the RT reaction that are also used in the qPCR reaction, instead of separate, downstream RT primers. RT-qPCR measurements were performed on a Lightcycler 480 (Roche) with Sensifast SYBR Green master mix (Bioline) according to manufacturer's instructions. Expression levels of genes of interest were quantified using the LinregPCR program (Ruijter, Ramakers et al. 2009) and normalized for 18S rRNA levels determined on 1000× diluted templates. Normalized mRNA levels are expressed as a percentage of the mean mRNA levels of two human liver samples normalized to 18S rRNA. The human liver samples were obtained from two female patients aged 40 and 41 with liver adenoma and no elevated liver damage. These patients were not on medication and had no history of drug or alcohol abuse. The samples were taken after obtaining written informed consent and the procedure was approved by the Academic Medical Center's committee on human experimentation (protocol number 03/024).

Mitochondrial Content

To assess the cellular mitochondrial content the inventors determined the mitochondrial/nuclear DNA ratio. Total DNA was isolated with the QIAamp DNA Mini Blood kit (QIAgen) according to manufacturer's instructions. Mitochondrial/nuclear DNA ratio was analyzed via qPCR on a Lightcycler 480 (Roche) with SensiFAST SYBR Green master mix as described (Hoekstra, Deurholt et al. 2005).

Expression levels of genes of interest (mitochondrial encoded genes Cytochrome c oxidase subunit III (mtCO3) and NADH dehydrogenase (mtND1) (McGill, Sharpe et al. 2012) and nuclear encoded genes CCAAT/enhancer binding protein alpha (CEBPA) and N-acetyltransferase 1 (NAT1)) were quantified using the LinregPCR program (Ruijter, Ramakers et al. 2009). For a list of PCR-primers used, see supplementary table 1.

Bilirubin Glucuronidation

Bilirubin glucuronidation was determined in medium and in cell samples. First, cells were incubated in FBS-free and phenol red-free HepaRG medium containing 10 μM bilirubin (mixed isomers, B-4126, Sigma) for 0, 1, or 4 h. At the 0 h time point the cells were incubated for 5 seconds to correct for non-specific binding of bilirubin to the cells and the culture plate. Medium samples were immediately stored at −80° C. For measurements of intracellular bilirubin glucuronidation, bilirubin-exposed cells were scraped into ice-cold PBS and pestle-homogenized on ice (30×, tight pestle). Samples of 25 μl containing 20 μg protein were incubated for 1 h at 37° C. together with 75 μl bilirubin incubation solution (50 mM Tris-HCl pH 7.8, 5 mM MgCl₂, 1 mM D-saccharic acid 1,4-lactone, 50 μM bilirubin, 3.5 mM uridine 5′diphospho-glucuronic acid (UDGPA), 2.5 mg/ml 1,2-dioleoyl-sn-glycerol-3-phosphocholine). The reaction was stopped by incubating in 2 volumes of methanol for 10 minutes on ice. The cell homogenate samples were stored at −80° C.

Prior to analysis medium and cell homogenate samples were thawed on ice, deproteinized with 2 volumes of methanol, and centrifuged for 5 minutes at 20000 g at 4° C. Supernatants were analyzed for bilirubin and bilirubin-conjugates by high-performance liquid chromatography (HPLC). Reverse-phase HPLC detection of bilirubin and its conjugates was adapted from a method described previously (Spivak and Carey 1985). Briefly, 100 μl of methanol deproteinized sample was applied to a Pursuit C18, 5 μm, 10 cm HPLC column (Varian, Palo Alto, Calif.). Starting eluent consisted of 50% methanol/50% ammonium acetate (1%, pH 4.5), followed by a linear gradient to 100% methanol in 20 minutes. Detection of bilirubin was performed at A=450 nm. Quantification of bi-, mono-, and unconjugated bilirubin was done by using a calibration curve of unconjugated bilirubin.

CYP Induction

Transcript levels of CYP genes were compared in cultures with and without induction. Cells were induced with omeprazole (40 μM, stock solution dissolved in DMSO), CITCO (1 μM, stock solution dissolved in DMSO) or rifampicin (4 μM, stock solution dissolved in DMSO) with a final concentration of 0.1% DMSO in FBS-free and phenol red-free HepaRG medium for 24 h after which total RNA was isolated immediately.

CYP Activity

Cells were incubated in FBS-free and phenol red-free HepaRG medium with the following drugs for 5 h: bupropion (100 μM, 50 mM stock dissolved in ethanol), phenacetin (200 μM, 100 mM stock in ethanol), tolbutamide (100 μM, 100 mM stock dissolved in ethanol), dextromethorphan (40 μM, 40 mM stock in water), chlorzoxazone (100 μM, 100 mM stock in DMSO), testosterone for 1 h (200 μM, 200 mM stock in ethanol) after which the medium was harvested and stored at −80° C. prior to analysis.

Frozen samples were thawed at room temperature, diluted with a solution containing metabolites with stable isotopes or diluted with 0.1% formic acid in ultrapure water (for 6β-OH-testosterone and OH-chlorzoxazone). CYP450 metabolites were quantified by HPLC tandem mass spectrometry. The system consisted of an AB Sciex (Framingham, U.S.A) API3200 triple quadrupole mass spectrometer working in electrospray ionization mode, interfaced with an Agilent (Santa Clara, U.S.A) 1200SL HPLC. Chromatography was performed at 70° C. with 10 μl injected into a Zorbax Eclipse XDB C18 column (50 mm×4.6 mm, 1.8 μm particle size), at a flow rate of 1.5 ml/min. The mobile phase was 0.1% formic acid in ultrapure water (A) and 0.3% formic acid in a mixture of methanol and acetonitrile (B). The proportion of the mobile phase B was increased linearly from 0 to 98% in 3 min, and then the column was allowed to re-equilibrate at the initial conditions. The total run time was 5 min. For 6β-OH-Testosterone, the mobile phase was ammonium acetate 5 mM in ultrapure water (A) and 0.3% formic acid in a mixture of methanol and acetonitrile (B). The proportion of the mobile phase B was increased linearly from 30 to 37% in 2.8 min, and then, after 1 min at 99% of B, the column was allowed to re-equilibrate at the initial conditions. The total run time was 5 min. For OH-chlorzoxazone, the mobile phase was 0.01% formic acid in ultrapure water (A) and acetonitrile (B). The proportion of the mobile phase B was increased linearly from 10 to 50% in 1.2 min, and then the column was flushed with 95% of the mobile phase B and the allowed to re-equilibrate at the initial conditions. The total run time was 3.0 min. The column eluent was split to an electrospray ionization interface, operating at 650° C. in both modes operating in multiple reaction monitoring mode.

In addition, CYP3A4 activity was quantified with CYP3A4 P450-Glo™ Assays (Promega) according to the manufacturer's instructions. The CYP activities were normalized for total protein, measured as indicated above.

Drug-Induced Toxicity

Cells were incubated in phenol red-free HepaRG medium with amiodarone (stock solution dissolved in DMSO, total concentration of 0.2% DMSO during incubation), acetaminophen (dissolved directly into culture medium), indomethacin (stock solution dissolved in DMSO, total concentration of 1% DMSO during incubation) or dextromethorphan (stock solution dissolved in DMSO, total concentration of 0.1% DMSO during incubation) with the indicated fold Cₘₐₓ (Xu, Henstock et al. 2008) for 24 h after which ATP levels were assessed with the CellTiter-Glo Luminescent Cell Viability Assay kit (Promega) according to manufacturer's instructions. Cₘₐₓ is defined as the therapeutically active average plasma maximum concentration, TC50 is defined as the concentration at which the inventors observed a 50% decrease in total ATP levels.

Low Clearance Compounds

Cells were incubated in a 24 wells plate in 500 μl/well FBS-free and phenol red-free HepaRG medium with 1 μM warfarin (stock solution dissolved in DMSO), theophylline (stock solution dissolved in DMSO), or prednisolone (stock solution dissolved in DMSO) with a final concentration of 0.01% DMSO for 1, 4, 8, or 24 h. At the 0 h time point the cells were incubated for 5 s to correct for non-specific binding of the added compounds to the cells and the culture plate. Medium samples of the indicated time points were immediately frozen at −80° C. Prior to HPLC analysis samples were thawed on ice and deproteinized with the addition of 4 volumes of acetonitrile (for prednisolone and theophylline), vacuum evaporated and dissolved in water. Warfarin samples were instead deproteinized with 2 volumes of methanol and spun down for 5 minutes at 20000 g at 4° C. Supernatants were used for HPLC analysis.

Reverse-phase HPLC detection was done as follows. Deproteinized samples (100 μl for prednisolone and theophylline, 30 μl for warfarin) were applied to a Hypersil C18, 3 μm, 15 cm HPLC column (Thermo Scientific). Starting eluent consisted of 6.8 mM ammoniumformate (pH 3.9), followed by several steps of linear gradients to different concentrations of acetonitrile (ACN) (Biosolve, Valkenswaard, The Netherlands). For prednisolone: 0 min 0% ACN, 1 min 0% ACN, 7 min 30% ACN, 17 min 36% ACN, 18 min 60% ACN, 19 min 60% ACN, 19.5 min 0% ACN, and 25 min 0% ACN. For theophylline and warfarin: 0 min 0% ACN, 1 min 0% ACN, 15 min 60% ACN, 19 min 60% ACN, 19.5 min 0% ACN, and 25 min 0% ACN. Detection of prednisolone was performed at λ_{abs}=254 nm, theophylline at λ_{abs}=270 nm, warfarin at λ_{exc}=310 nm/λₑₘ=390 nm. Quantification was done by using calibration curves of prednisolone, theophylline, or warfarin.

In vitro intrinsic clearance (CLᵢₙₜ) was calculated from compound loss according to using Eq. 1 and 0.45×10⁶ cells/well.

$${CL}_{int} ( μl \cdot {min}^{- 1} \cdot {10}^{6} {cells}^{- 1} ) = \frac{V \times 0.693}{{t}_{1 / 2}} \\ where \\ V ( μl \cdot {10}^{6} cells ) = \frac{incubation volume ( μl )}{number of cells in incubation ( \times {10}^{6} )} \\ Half life ( {t}_{1 / 2} ) ( min ) = \frac{0.693}{k} \\ Elimination rate constant ( k ) = - slope of ln ( \% drug remaining vs time ) \qquad Eq . 1$$

Malaria Infection

Malaria-(Plasmodium falciparum, Pf) infected mosquitoes (Anopheles stephensi) were dissected under the microscope by hand. The salivary glands were crushed and the number of spozoroites (Spz) were counted using a hemocytometer.

Monolayer HepaRGs in 12 well plates contained 1 ml of medium.

5×10⁴Spz (either alive or dead, heat-killed, as negative control) were added to each well containing HepaRG cells, after which the cells with Pf-Spz were spun down 5-10 minutes at 1100 rpm. Monolayers were then transferred to a 37° C. cell incubator, without shaking. The next morning the baliads were transferred to 12-well plates with 1 ml medium on an orbital microplate shaker (VWR) at 100 rpm.

At day 1, 3, and 5 after infections cells were washed twice with PBS. The HepaRG monolayers were harvested by adding 0.3 ml of trypsin, placing in incubator for 5 minutes. 0.7 ml of complete medium was added to inactivate trypsin. Accumax was used for the HepaRG baliads. Each baliad was treated with 2 times 1 ml accumax. Each time after adding accumax the cells were transferred to the incubator for 5 minutes. After each incubation accumax was pipetted over the baliads 15 times. Monolayer and baliads cells were spun down 1 minute at 10.000 rpm.

The pellet was used for RNA isolation, using RNeasy mini kit (Qiagen), after which cDNA was synthesised using 9 μl of RNA and gene specific primers against Pf 18S ribosomal RNA. A 1:100 dilution was made from each cDNA sample. qRT-PCR was performed on a CFX96 Real-time C1000 thermal cycler (Bio-Rad). Data was analysed using Bio-Rad CFX manager and Graphpad Prism.

Statistics

Data are expressed as mean±SD and were calculated with Prism 6.07 (Graphpad). Statistical significance was determined by performing a Student's T-test or two-way ANOVA with Tukey's post-hoc correction for multiple testing and is indicated in the legend of the figures. Graphs were plotted with Prism 6.07.

Results

Overexpression of CAR in HepaRG Cells Alters Morphology During Culture with DMSO

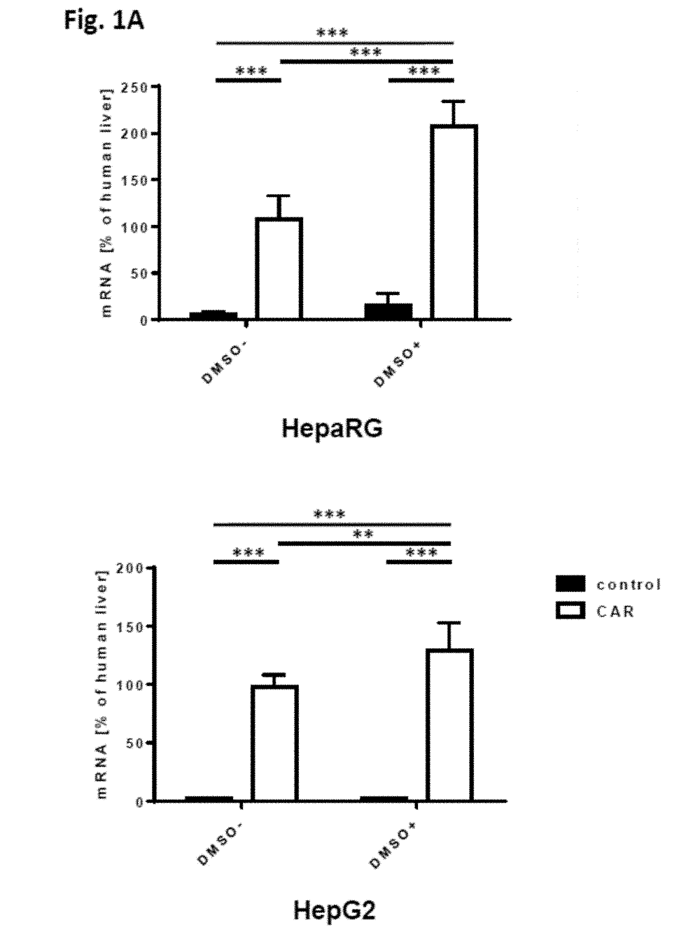

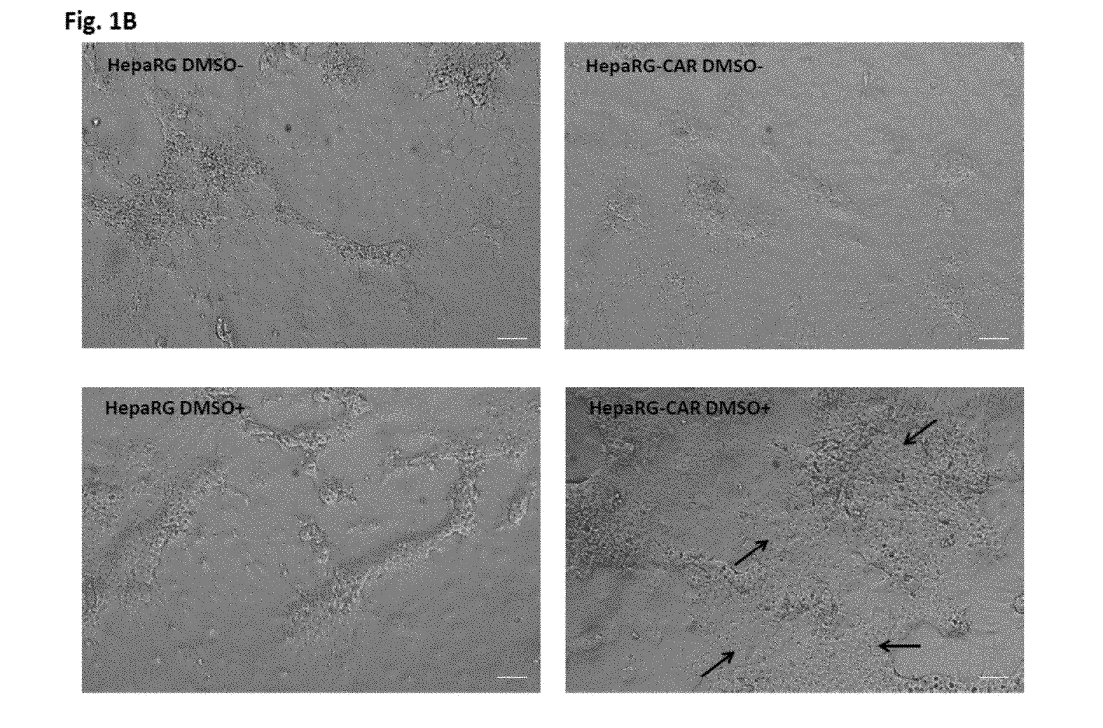

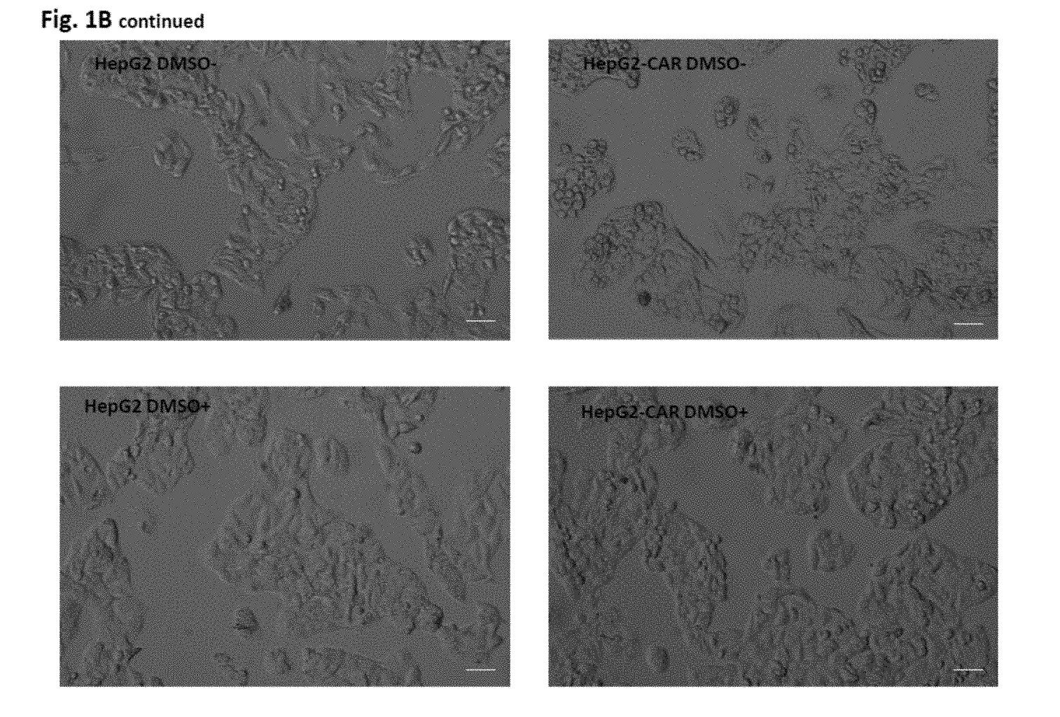

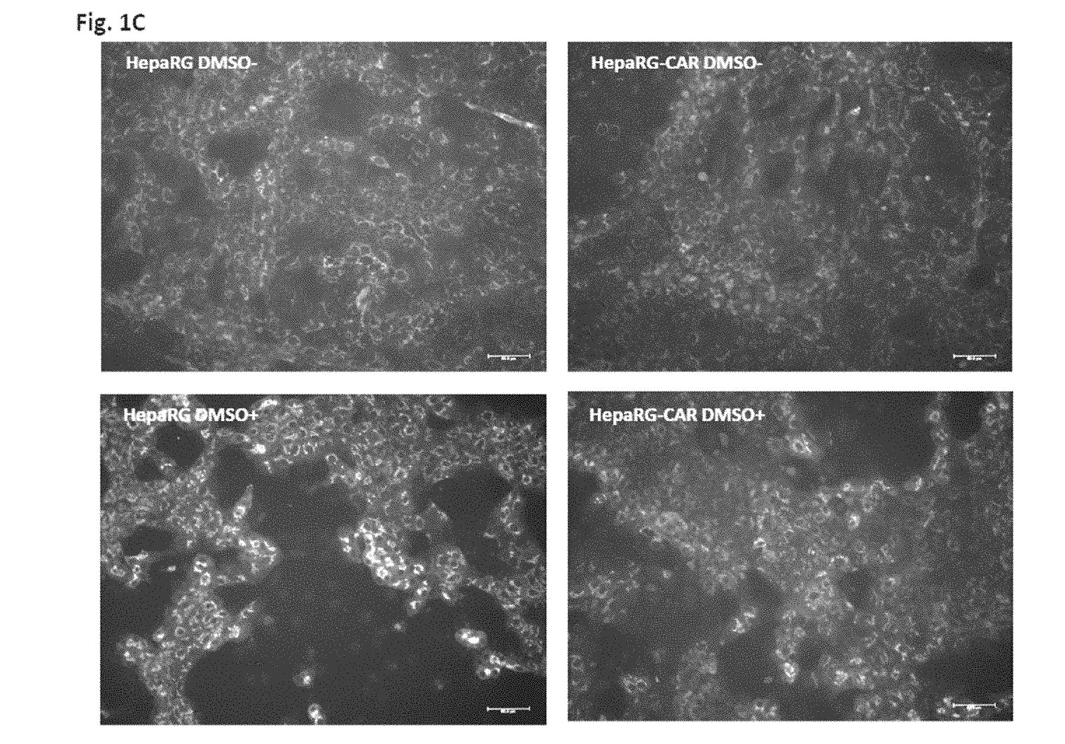

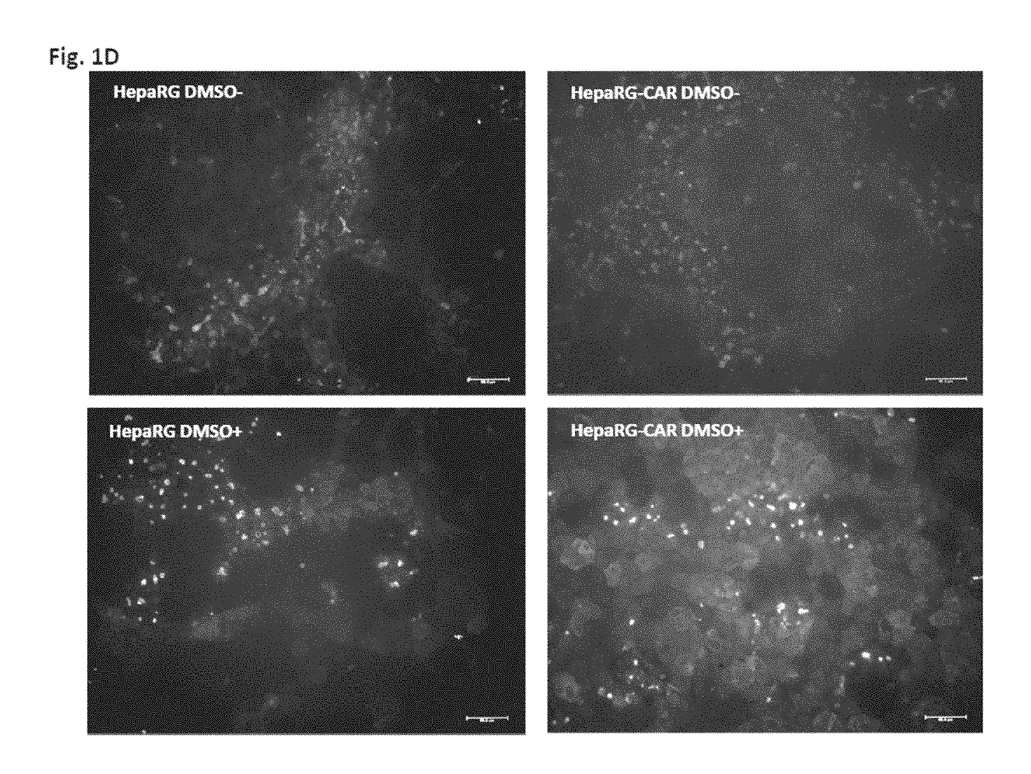

To investigate the role of CAR in the function and differentiation of HepaRG cells, the inventors generated a stable cell line overexpressing CAR in HepaRG cells via lentiviral transduction, which the inventors named HepaRG-CAR. Compared to the average of two human liver samples, CAR mRNA levels were increased from 5.3% in control HepaRG cells to 108% in modified HepaRG cells, both cultured without DMSO (FIG. 1A). Increased expression of CAR was stable over at least 7 passages (not shown). Culture with DMSO increased CAR mRNA levels to 15% of human liver in control HepaRG cells and to 208% in modified HepaRG cells (FIG. 1A). HepaRG cells normally differentiate into a 1:1 ratio of hepatocyte islands and cholangiocyte-like cells (Gripon, Rumin et al. 2002). Surprisingly, when cultured with DMSO, HepaRG-CAR cultures showed an altered morphology in which most of the cholangiocyte-like cells were absent and a new sub-set of hepatocyte-like cells were present in between the hepatocyte islands (inter-island hepatocyte-like cells) (FIG. 1B). Culture of modified HepaRG cells without DMSO resulted in no visible morphological alterations (FIG. 1B) compared to the parental HepaRG cells.

In order to determine the level of differentiation of this new sub-set of hepatocyte-like cells in HepaRG-CAR, the inventors examined the expression of albumin and the apical multidrug transporter MRP2 (ABCC2). Albumin was intensely stained in the cytoplasm and particularly peri-nuclear in both hepatocyte islands and cholangiocytes in control and modified HepaRG cells cultured without DMSO (FIG. 1C). In DMSO-treated control and HepaRG-CAR cultures hepatocyte islands were positive, however cholangiocyte-like cells were negative. In addition, inter-island hepatocytes in the HepaRG-CAR cultures expressed albumin (FIG. 1C). MRP2, a marker for hepatic polarization, was strongly stained in canalicular structures and channels in hepatocyte islands of both cell lines cultured with or without DMSO, although more intense staining could be observed in DMSO cultures (FIG. 1D). The inter-island hepatocyte-like cells in the DMSO-treated HepaRG-CAR cultures did not form MRP2-stained canaliculi. Diffuse intracellular staining of MRP2 was also clearly visible in DMSO-treated cells and in modified HepaRG cells cultured without DMSO (FIG. 1D).

Increased Expression of CAR Target Genes in Modified HepaRG Cells and Partly in HepG2-CAR Cells

The inventors determined the transcript levels of several genes in CAR-overexpressing HepaRG cells (table 1). Transcript levels of typical CAR targets were increased by CAR overexpression to different degrees, with most pronounced effects after treatment with DMSO: CYP286 (26×), CYP2C8 (2.8×), CYP2C9 (2.3×), CYP2C19 (3.0×), CYP3A4 (2.7×), UGT1A1 (6.2×), MRP2 (1.5×), and cytochrome P450 reductase (POR) (2.2×), however, the CYP1A2 transcript level was unchanged. Transcript levels of non-CAR target CYPs were unaltered (CYP2D6) or decreased (CYP2E1: 9.2×). Other tested hepatic genes were unaffected by CAR overexpression, including OATP1B1, OATP2B1, NTCP, MRP3, AOX1, CK7, HNF4α, FXR, AHR, and PXR.

In HepG2 DMSO cultures, overexpression of CAR increased transcript levels of the CAR targets CYP286 (9.0×) and UGT1A1 (4.5×) (table 2). However, CAR target CYP1A2 transcript level was decreased (2.3×). Omission of DMSO treatment yielded a similar pattern: CYP286 (6.2×) and UGT1A1 (6.7×). In contrast to HepaRG cells, HepG2 cells showed less response to CAR overexpression, as not all tested CAR target genes were increased in their transcript levels, including CYP2C9, CYP3A4 and POR. Non-CAR target CYPs were unaltered in their transcript levels (CYP2D6 and CYP2E1) during culture with DMSO in HepG2-CAR. Similar to HepaRG, transcript levels of other tested hepatic genes were unaffected by CAR overexpression in HepG2 cells: OATP2B1, NTCP, HNF4α, and PXR.

Induction Rates of AHR and CAR, but not PXR, are Unaffected by Overexpression of CAR in HepaRG Cells

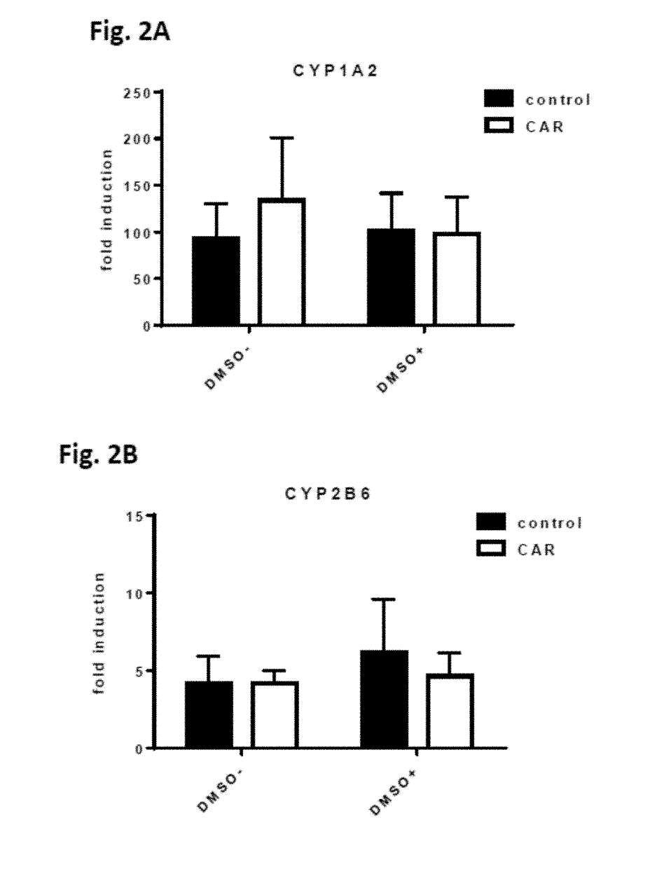

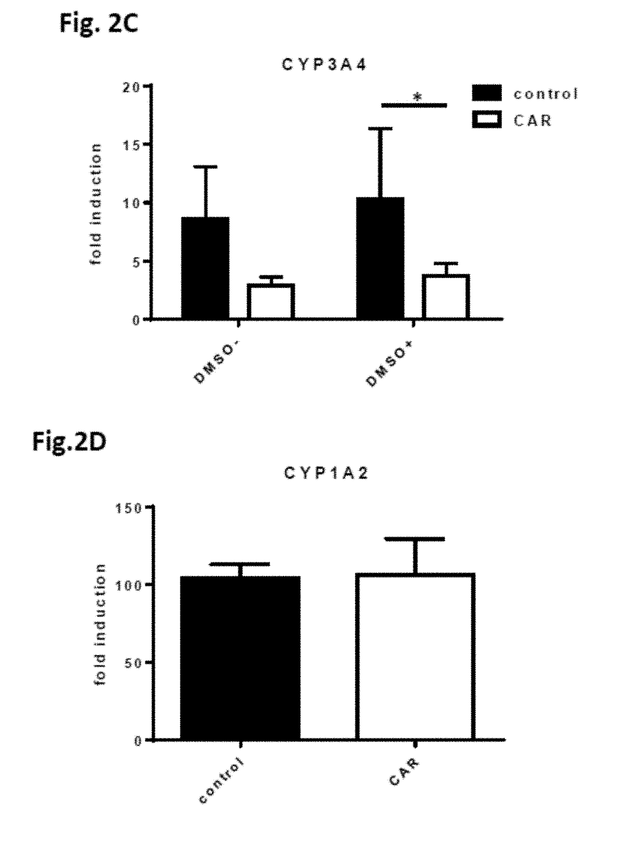

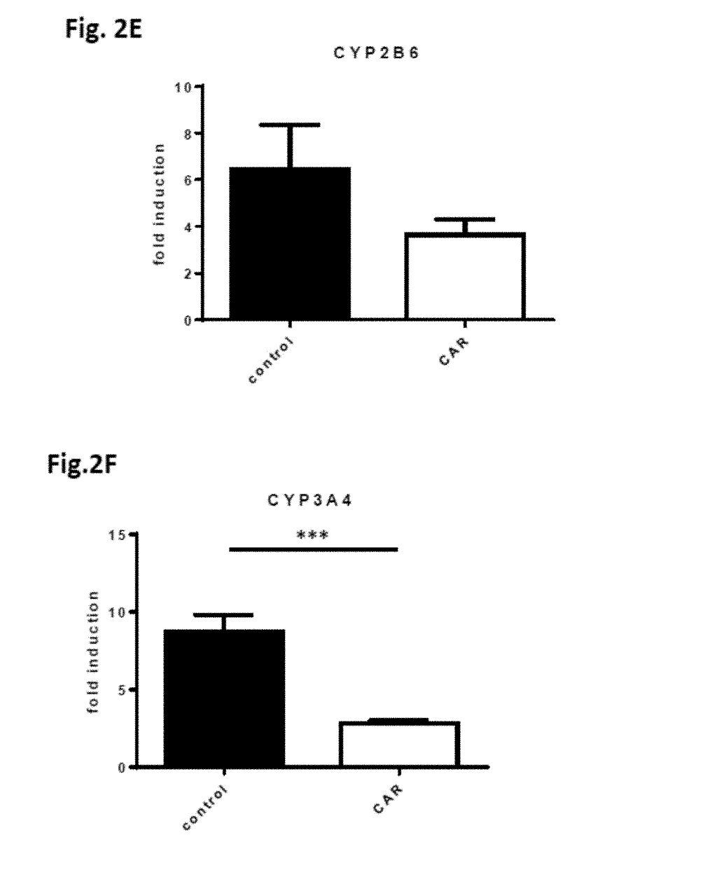

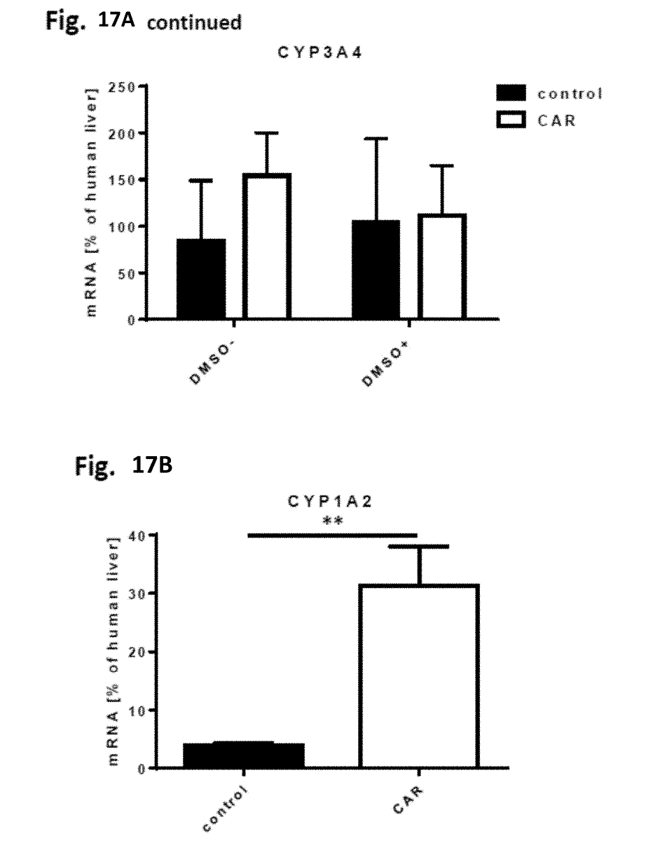

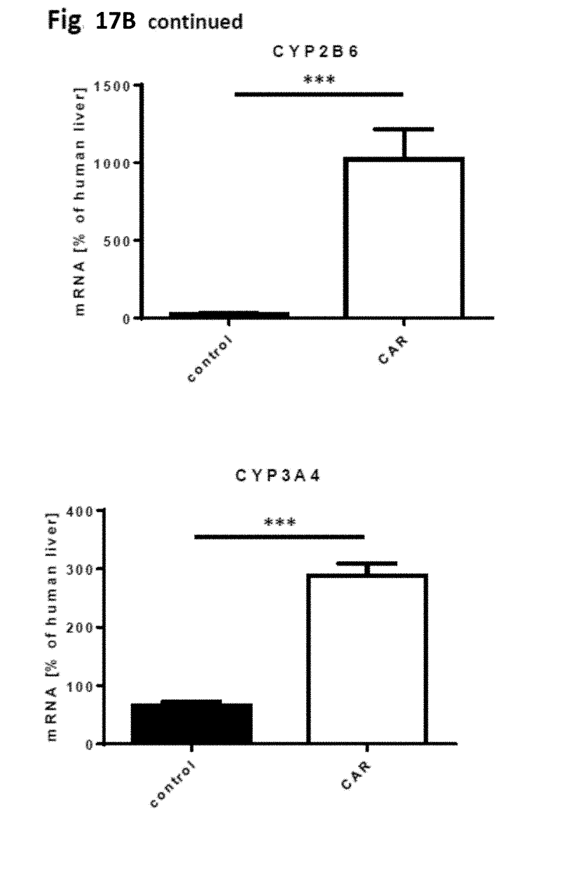

To determine if the induction of xenobiotic nuclear receptors would be preserved by overexpression of CAR, the inventors investigated the transcript levels of typical target CYPs of AHR, CAR and PXR after a 24h incubation with specific inducers: omeprazole for AHR, CITCO for CAR, and rifampicin for PXR. Addition of omeprazole and CITCO resulted in a similar fold induction of AHR target gene CYP1A2 (93-134×) and CAR target gene CYP2B6 (4.2-6.1×), respectively, in control and modified HepaRG cells, independent of DMSO treatment (FIGS. 2A and B). Interestingly, addition of rifampicin resulted in a reduced fold induction of CYP3A4 in modified HepaRG cells after DMSO treatment (3.6×) compared to control cells (10×) (FIG. 2C). After induction, total transcript levels of CYP1A2 and CYP2B6 remained higher in modified HepaRG cells compared to controls, up to the level of 23% (CYP1A2) and 430% (CYP2B6) of human liver, while CYP3A4 expression was equal between all groups (FIG. 17A). In BALIAD cultures (without DMSO), modified HepaRG cells showed a similar induction of CYP1A2 (106× vs 104×) and CYP2B6 (3.6× vs 6.5×), yet a decreased induction of CYP3A4 (2.8×vs 8.8×), when compared to control BALIADs (FIG. 2D-F). After induction total mRNA levels of CYP1A2, CYP2B6, and CYP3A4 were higher in modified HepaRG cells compared to controls (FIG. 17B) up to the level of 31% (CYP1A2), 1023% (CYP2B6), and 289% (CYP3A4) of human liver. Because of the omission of FBS from the culture medium during the induction experiments there is a discrepancy in CYP transcript levels with those presented in table 1.

Increased Activity of CAR- and Non-CAR Target CYPs in Modified HepaRG Cells

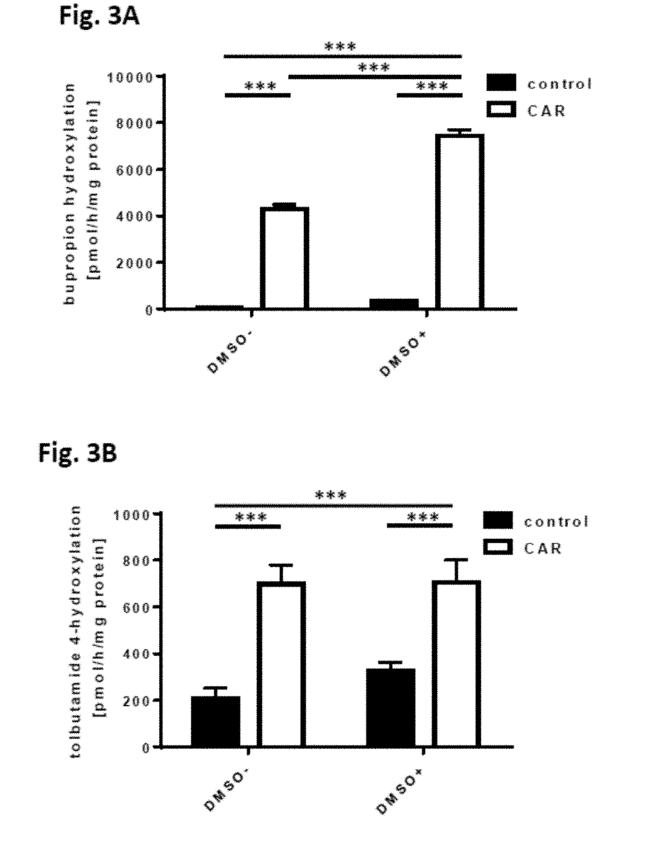

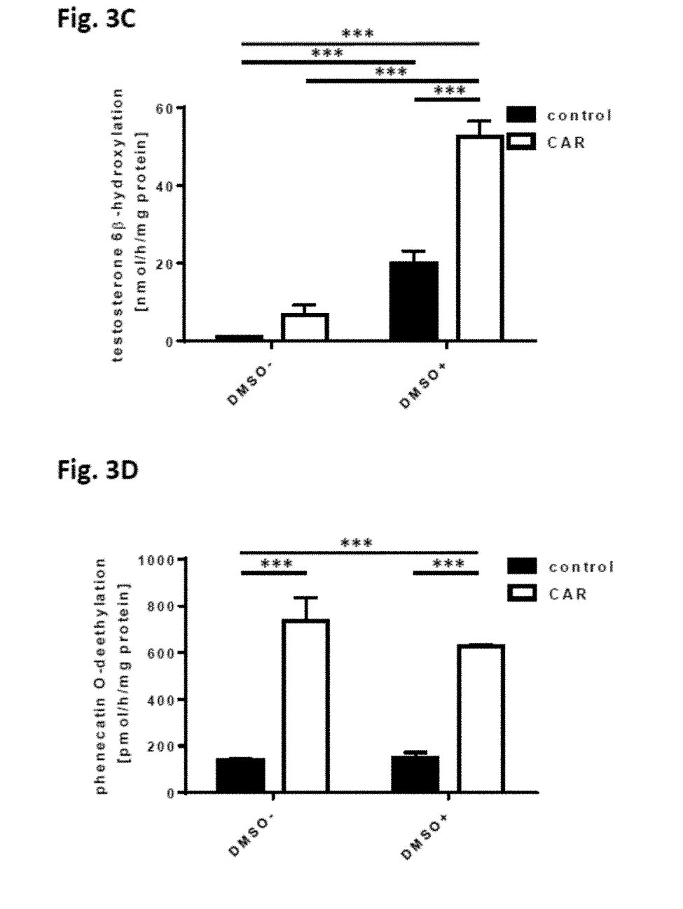

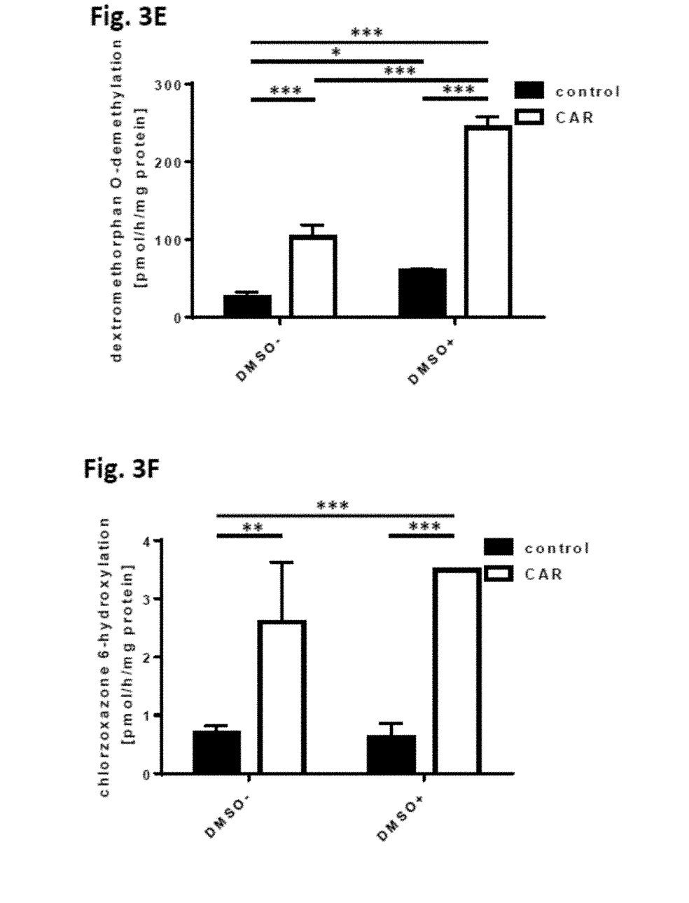

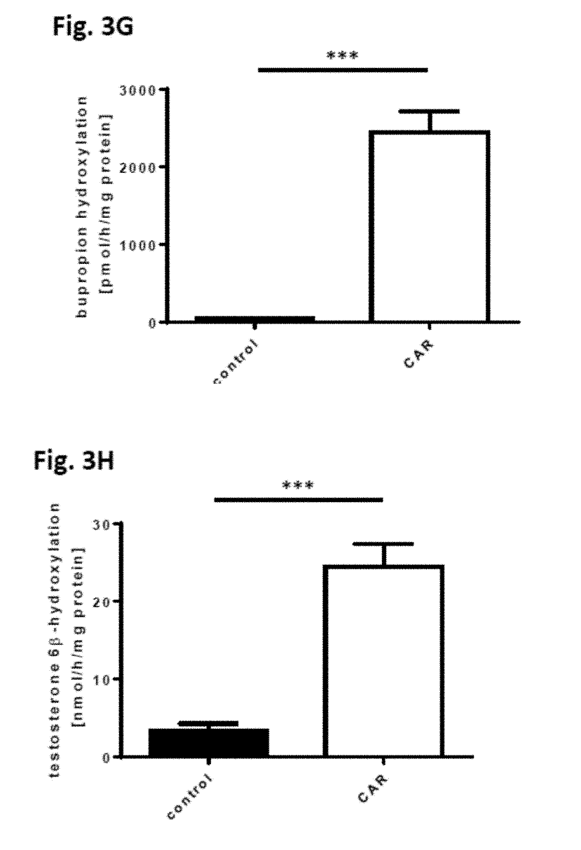

Next, the inventors assessed whether increased transcript levels of canonical CAR target CYPs also resulted in increased enzyme activity in modified HepaRG cells. Indeed, for all 4 tested CAR CYP targets the inventors observed increased activity in HepaRG-CAR compared to control: CYP1A2 (5.4×without DMSO, 4.9× with DMSO), CYP2B6 (52× without DMSO, 23× with DMSO), CYP2C9 (3.2×without DMSO, 2.9× with DMSO), and CYP3A4 (7.4× without DMSO, 2.9× with DMSO) (FIG. 3A-D). Unexpectedly, enzyme activities of non-CAR target CYPs were also increased by CAR overexpression, despite unchanged or reduced transcript levels. Activities of CYP2D6 (5.0× without DMSO, 4.1× with DMSO) and CYP2E1 (3.3× without DMSO, 5.9× with DMSO) were increased in modified HepaRG cells compared to control cells (FIG. 3E-F). CYP2B6 and CYP3A4 activities were also increased in HepaRG-CAR BALIAD cultures by respectively 49× and 7.3× compared to controls (FIGS. 3G and H).

Increased UGT1A1 Activity in Modified HepaRG Cells

To determine if phase 2 drug metabolism was also increased the inventors examined the activity of UGT1A1 by the accumulation of bilirubin glucuronides in the culture medium after bilirubin loading. Despite increased transcript levels of UGT1A1 upon CAR overexpression in HepaRG cells, the inventors did not observe an increased accumulation of bilirubin glucuronides in the medium (FIG. 4A). However, when determined in cell homogenates, to which a non-limiting amount of UDP-glucuronic acid (UDPGA) co-factor was added, bilirubin mono- and di-glucuronidation levels were increased in modified HepaRG cells compared to controls, by respectively 5.3× and 8.3× in cells cultured without DMSO and by respectively 7.7× and 12.8× in cells cultured with DMSO (FIG. 4B). This indicates that CAR overexpression increases UGT1A1 activity, however UDPGA levels, or transport of bilirubin and/or its conjugates over the plasma membrane limit the accumulation of bilirubin conjugates in the culture medium.

Decreased Toxicity of Acetaminophen, Amiodarone, and Indomethacin in Modified HepaRG Cells

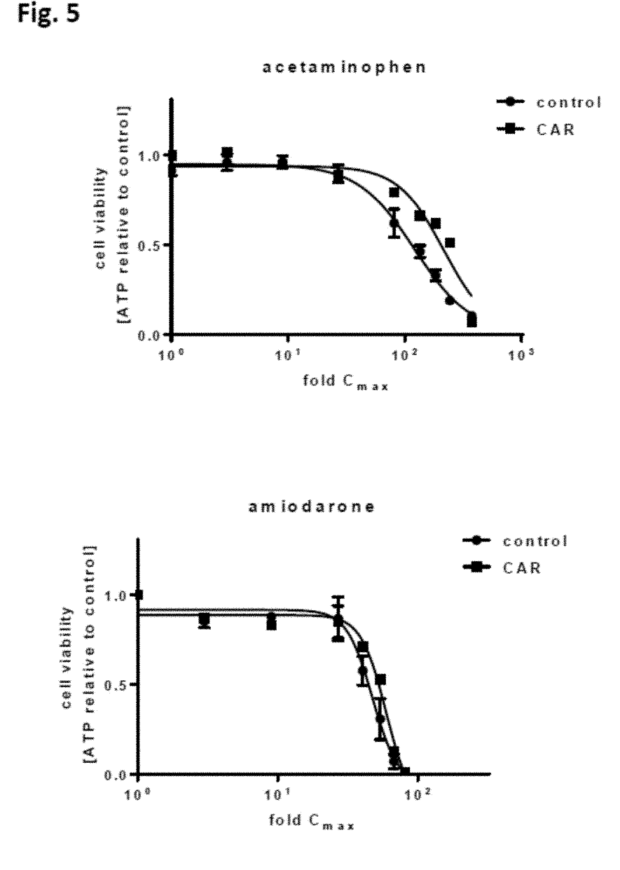

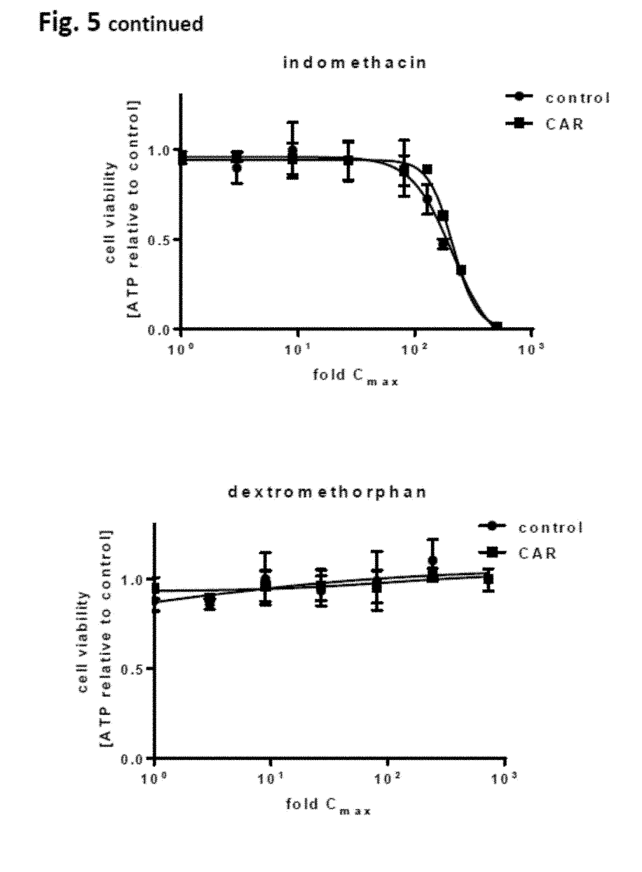

The increased expression and activity of CYPs in modified HepaRG cells should lead to faster metabolism and clearance of toxic concentrations of drugs. The inventors treated DMSO-cultured modified HepaRG cells and controls with several hepatotoxic drugs: acetaminophen, amiodarone, and indomethacin, while dextromethorphan was included as a non-hepatotoxic control (Ramaiahgari, den Braver et al. 2014). Indeed, TC50 values were significantly higher in modified HepaRG cells treated with acetaminophen or amiodarone compared to control cells (FIG. 5 and table 3). The TC50 values were not significantly different between the groups for indomethacin.

Increased Clearance of Warfarin and Prednisolone in Modified HepaRG Cells

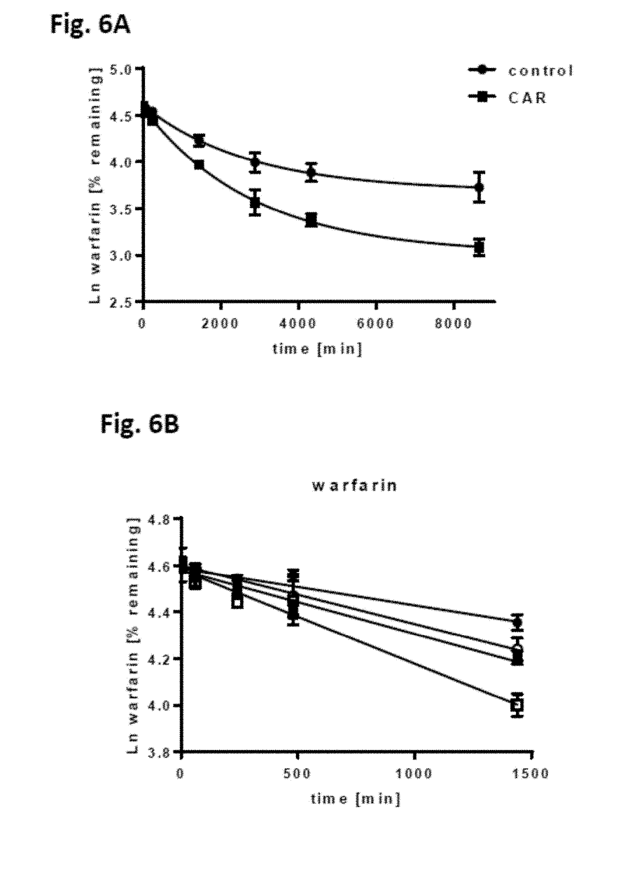

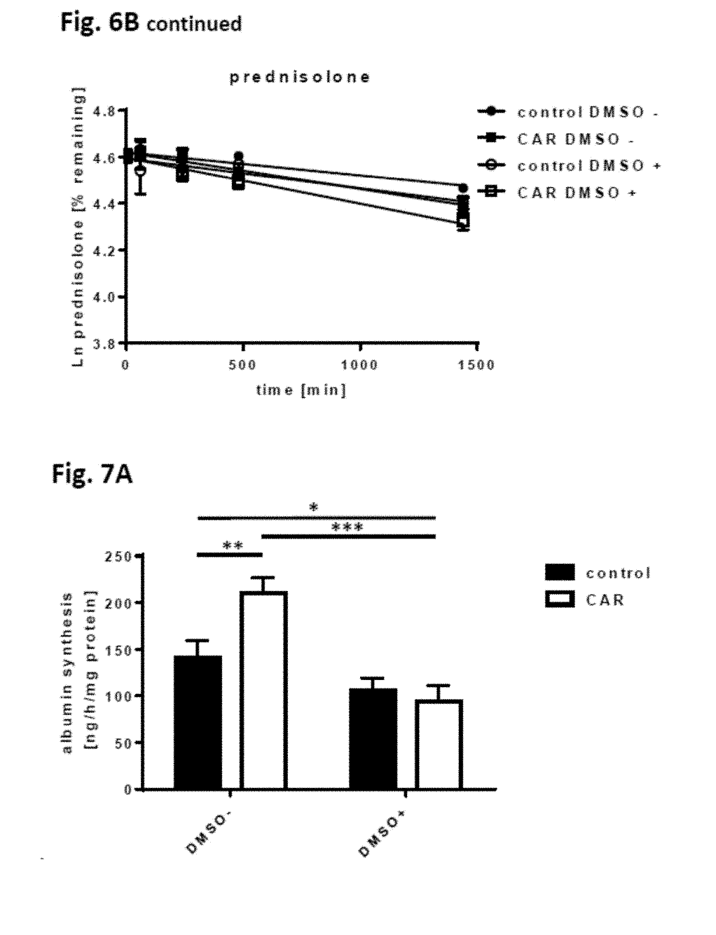

Since modified HepaRG cells have an increased rate of drug metabolism the inventors assessed their capability to clear three slowly metabolizable compounds: warfarin, theophylline, and prednisolone. Both HepaRG and modified HepaRG cells showed a linear clearance of warfarin during the first 24-48 h, which declined in a non-linear fashion afterwards (FIG. 6A). Therefore, the inventors assessed clearance of all three compounds from t=0-24 h. Both warfarin and prednisolone were cleared at an increased rate in HepaRG-CAR cultured with DMSO compared to all other conditions (FIG. 6B and table 4). Similar to other reports in literature the inventors could not reliably observe any clearance of theophylline (Bonn, Svanberg et al. 2016).

Limited or No Effect on Albumin Synthesis and Ammonia Elimination

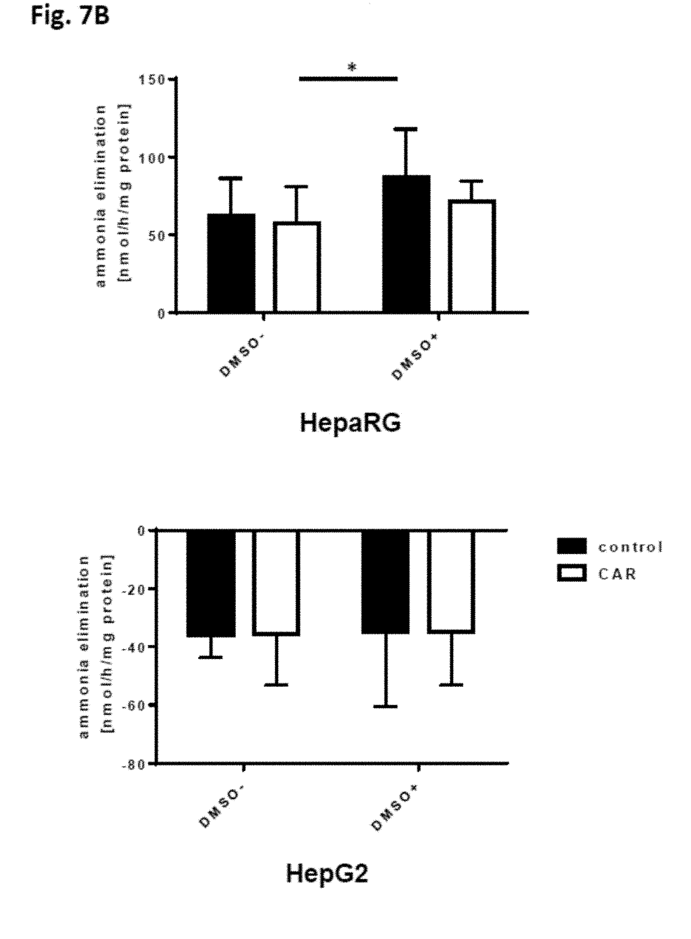

In order to determine the effect of CAR overexpression on hepatic activities unrelated to CAR the inventors assessed albumin production and ammonia elimination. Surprisingly, CAR overexpression in HepaRG cultures increased albumin synthesis by 49% in absence of DMSO, however in DMSO+ cultures CAR overexpression did not change albumin synthesis when compared with DMSO+ controls (FIG. 7A). Ammonia elimination was unchanged after CAR overexpression in both DMSO− and DMSO+ HepaRG cultures (FIG. 7B). HepG2 cells produced ammonia instead of eliminating it. The ammonia production was unchanged in HepG2 cells after CAR overexpression with or without DMSO treatment (FIG. 7B).

Increased Viability of Modified HepaRG Cells During Culture with DMSO, but Unaltered in HepG2-CAR Cells

Addition of DMSO induced cell death in control HepaRG cells, as judged from the 40% reduction of protein content, similar to previously reported (Hoekstra, Nibourg et al. 2011) (FIG. 8A). Modified HepaRG cells were less affected by DMSO and only showed a 19% reduction of total protein content when compared with untreated cells (FIG. 8A). Cellular integrity, assessed by AST leakage, and total ATP content were similar between control HepaRG and modified HepaRG cells, as measured in −DMSO medium for 24h (FIGS. 8B-C). However, WST activity corrected for protein, as a marker for cellular NADH levels, was 40% increased by CAR overexpression (FIG. 8D). Total protein content in HepG2 cells and HepG2-CAR cells was equally affected by DMSO treatment, with a reduction of 15%-18%. (FIG. 8A). Protein-normalized WST activity in HepG2-CAR cells was unchanged after culture with or without DMSO (FIG. 8D).

HepaRG Culture in 8AL-in-a-Dish Increases Expression of Hepatic Genes

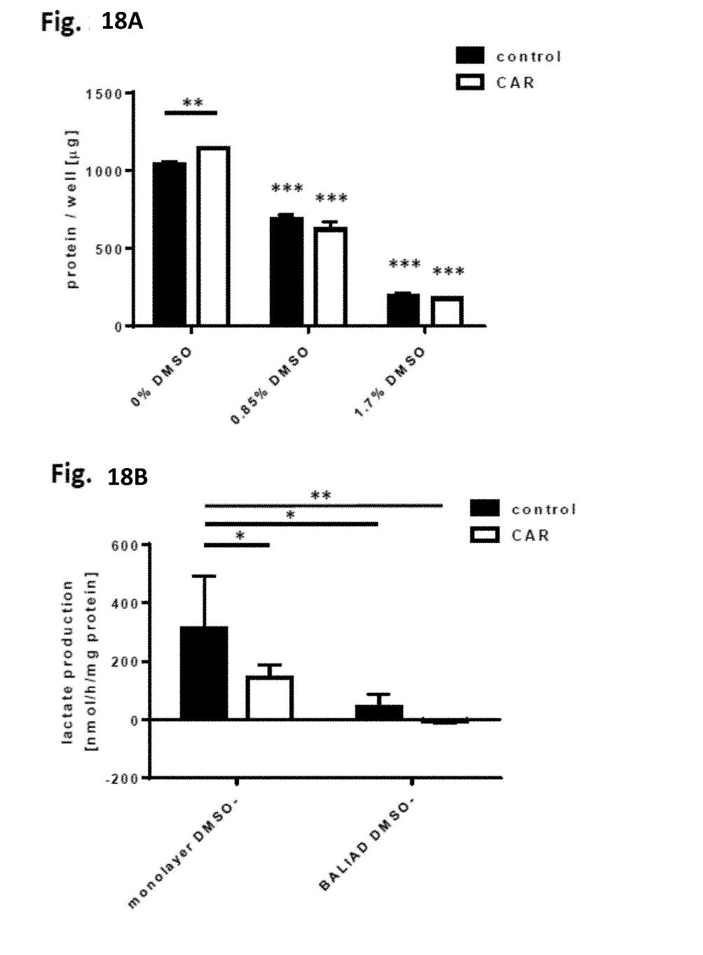

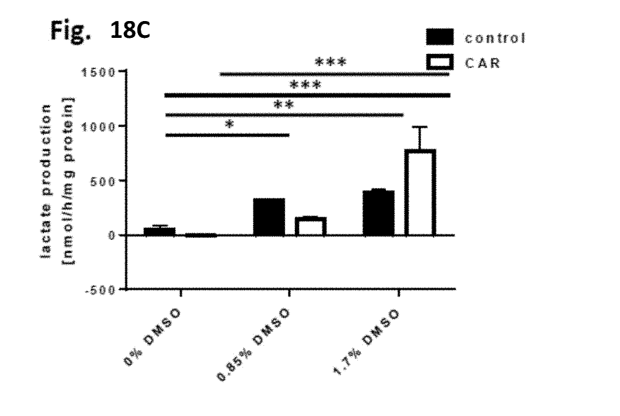

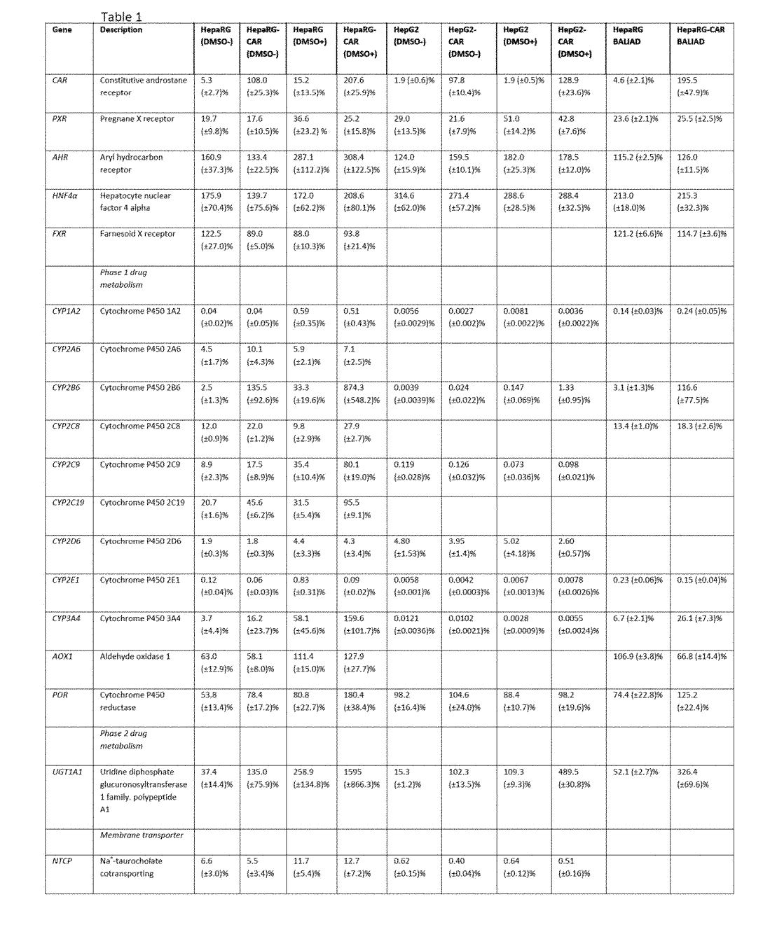

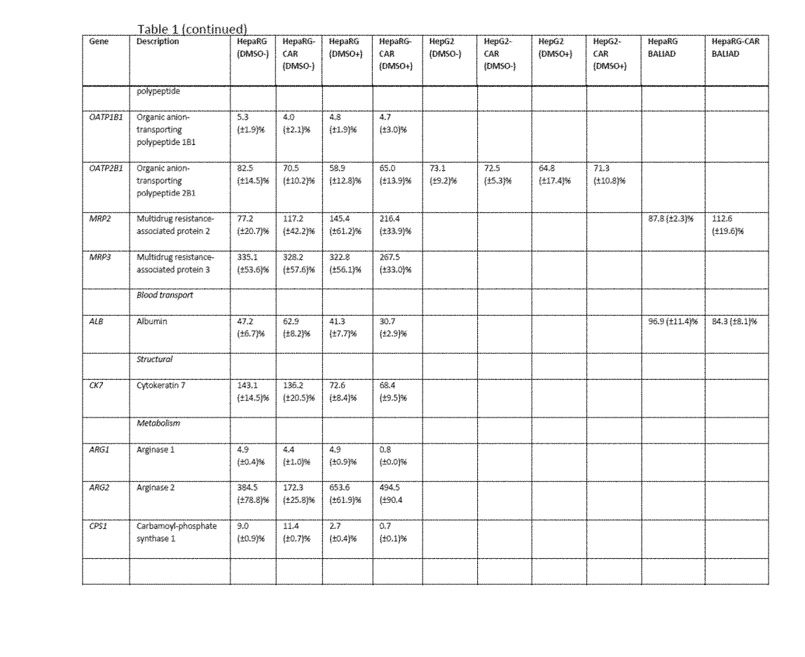

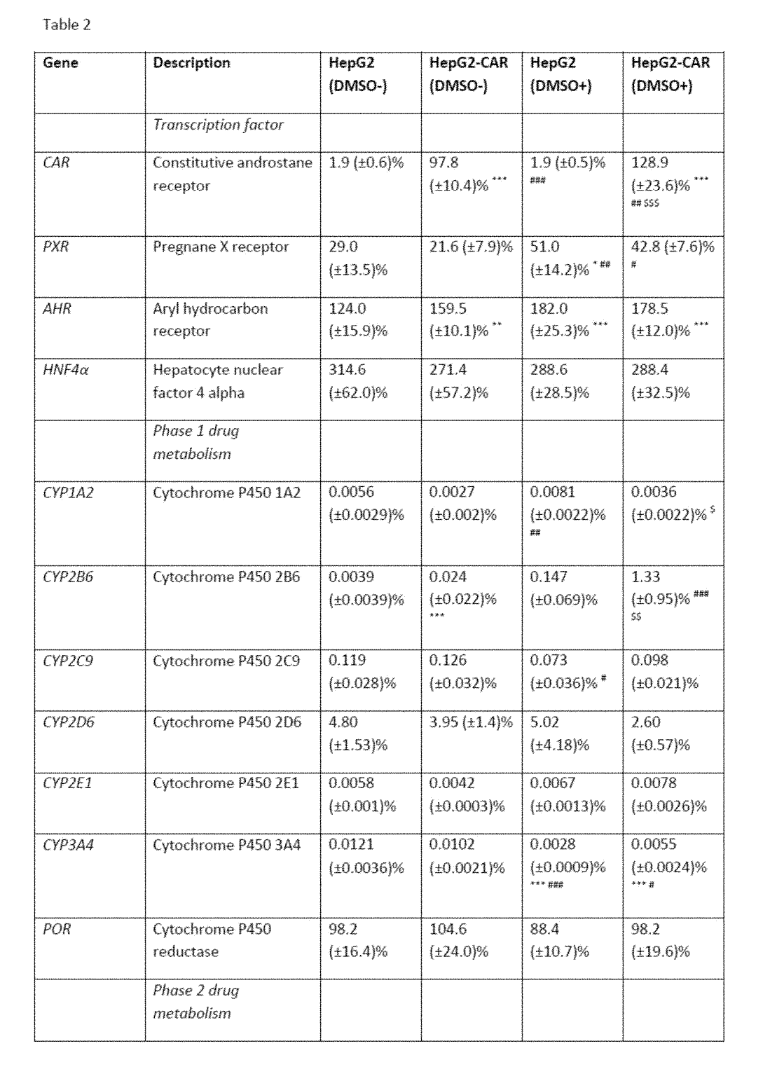

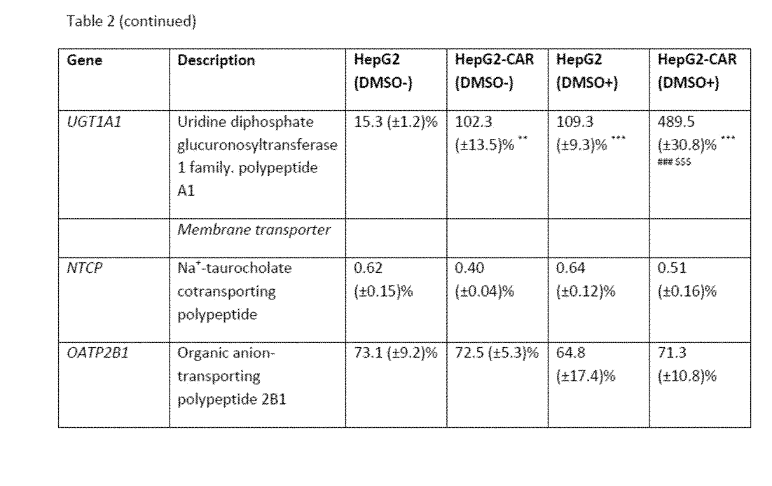

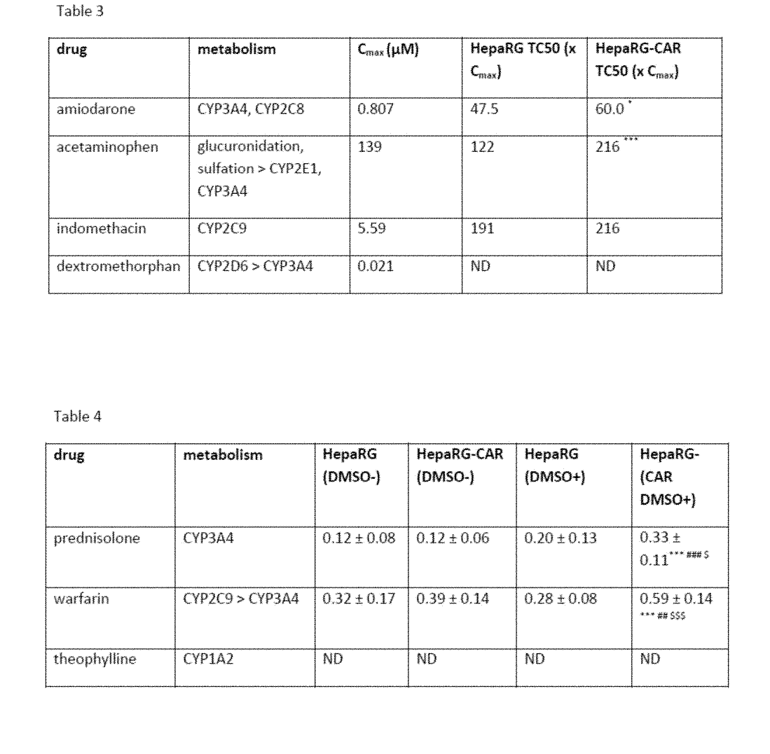

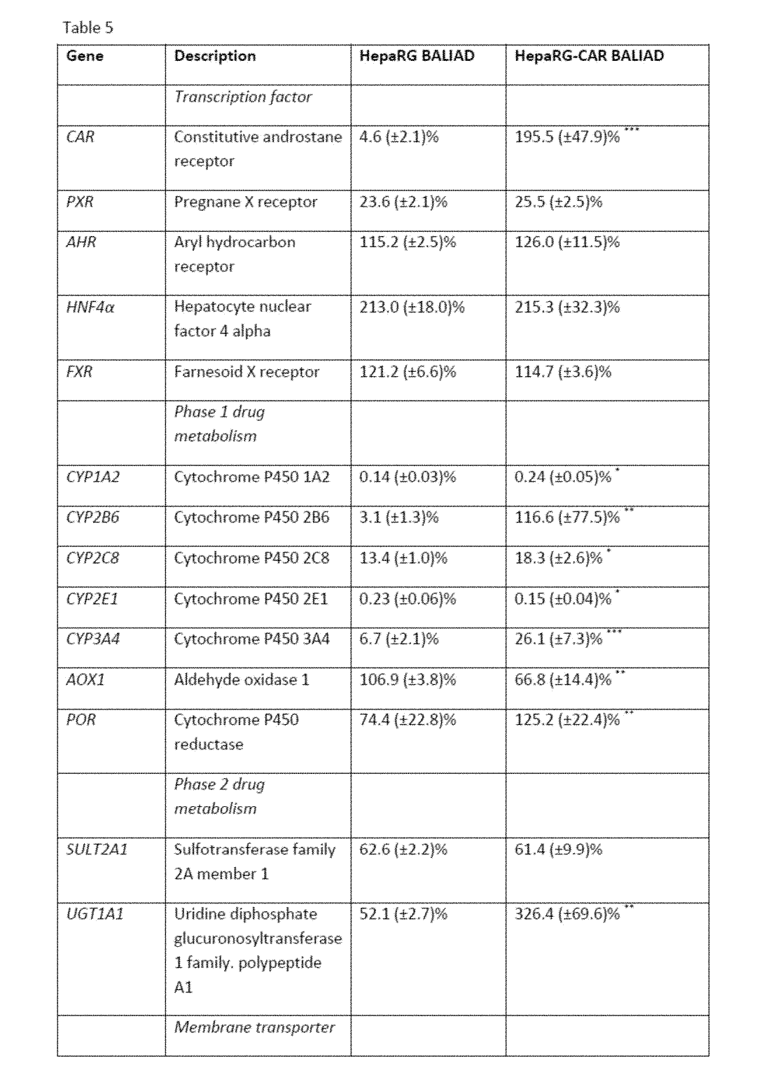

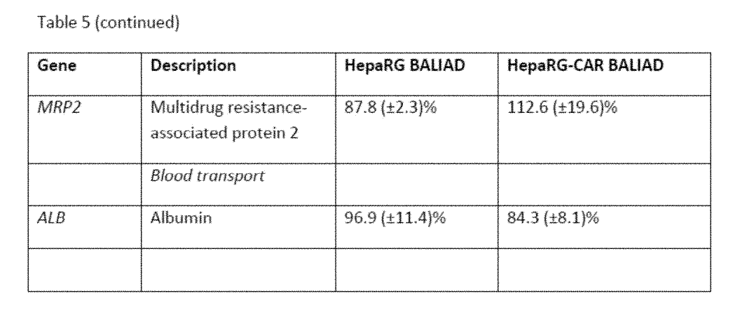

Interestingly, BALIAD cultures of HepaRG cells showed high susceptibility to DMSO-toxicity, independent of CAR overexpression, as evidenced by a 82% and a 84% decrease in total protein content in control cells and modified HepaRG cells, respectively (FIG. 18A). Culture with a reduced concentration of DMSO (0.85%) was toxic to a lower extent in both control cells and modified HepaRG cells with a reduction in total protein content of 34% and 46%, respectively (FIG. 18A). Thus, unlike HepaRG-CAR monolayer cultures, BALIAD cultures were not resistant to the toxic effects of DMSO.

The inventors then analyzed the transcript levels of several liver-specific genes in HepaRG+/− CAR cells cultured in the BALIAD system without DMSO (table 5). As in monolayers CAR overexpression induced all tested CAR target genes in BALIAD cultures compared to controls: CYP2B6 (38×), CYP2C8 (1.4×), CYP3A4 (3.9×), UGT1A1 (6.3×), and POR (1.7×). The transcript level of non-CAR target CYP1A2 was increased 1.7×, while non-CAR targets CYP2E1 and AOX1 were decreased by respectively 1.5× and 1.6× in HepaRG-CAR BALIADs. Other hepatic non-CAR target genes were unaffected in HepaRG-CAR BALIAD cultures: SULT2A1, HNF4α, FXR, AHR, PXR, MRP2, and ALB. In comparison with HepaRG or modified HepaRG cells cultured in monolayer without DMSO BALIAD culture improved the expression of several genes; for HepaRG cells: ALB (2.1×) and AOX1 (1.7×); for modified HepaRG cells: CAR (1.8×), POR (1.6×), and UGT1A1 (2.4×); for both HepaRG and modified HepaRG cells: CYP1A2 (3.4× and 5.5×) and CYP2E1 (1.9× and 2.5×).

Altered Energy Metabolism in HepaRG-CAR, but not in HepG2-CAR

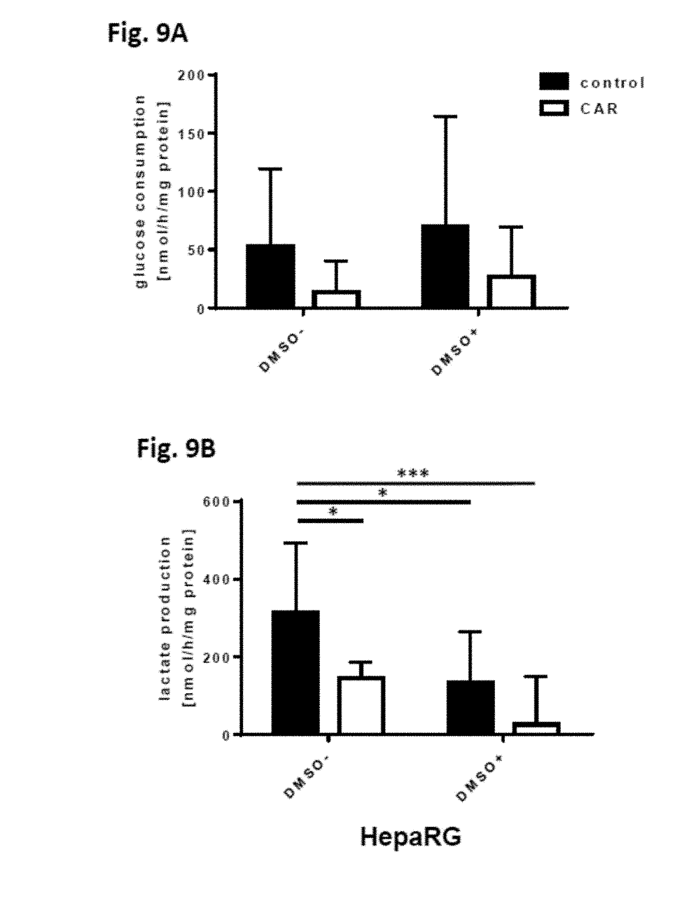

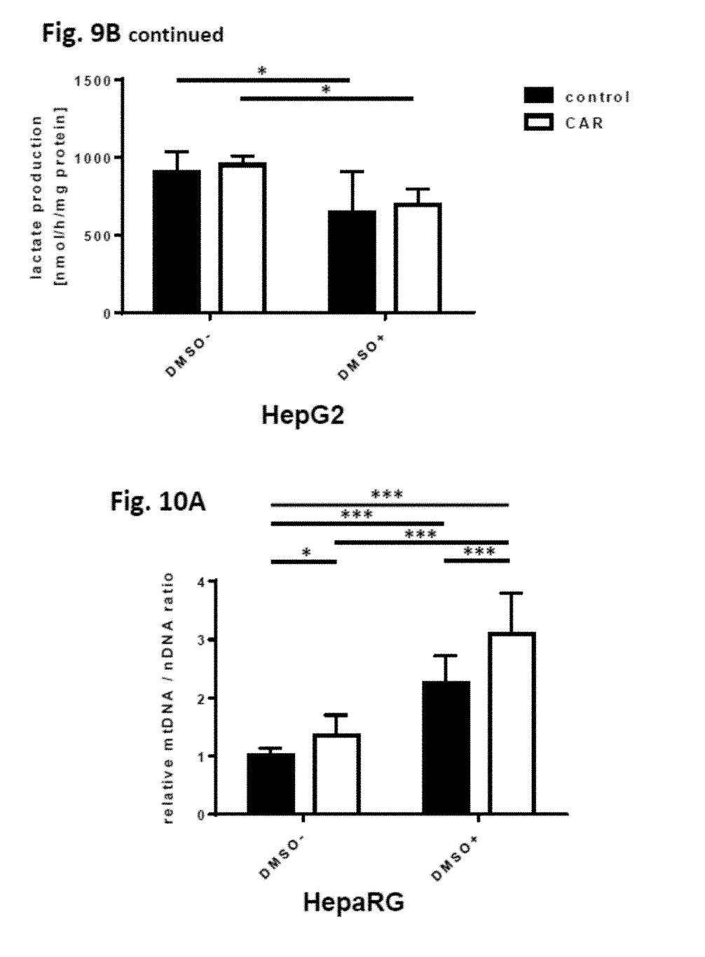

Since CAR is involved in glucose homeostasis, the inventors determined the effect of CAR overexpression on glucose consumption in HepaRG cells. There was a clear trend towards less glucose consumption in DMSO+/− cultures, however significance was not reached (FIG. 9A). Moreover, the modified HepaRG cells produced 54% less lactate than control cells when cultured without DMSO (FIG. 9B). Interestingly, culture with DMSO provided an additive effect and reduced lactate production by 57% in control cells and by 92% in HepaRG-CAR when compared to control cells cultured without DMSO (FIG. 9B). In contrast, CAR overexpression did not affect lactate metabolism in HepG2 cells, independent of DMSO addition (FIG. 9B). When cultured without DMSO, HepaRG or modified HepaRG cells cultured in the BALIAD produced less lactate compared to monolayer grown cells (FIG. 18B). Interestingly, modified HepaRG cells cultured without DMSO were even able to eliminate lactate instead of producing it. Culture with increasing concentrations of DMSO in BALIADs induced increasingly higher lactate production in both HepaRG and modified HepaRG cell lines (FIG. 18C).

Increased Mitochondrial Content in HepaRG-CAR and BALIAD Cultures

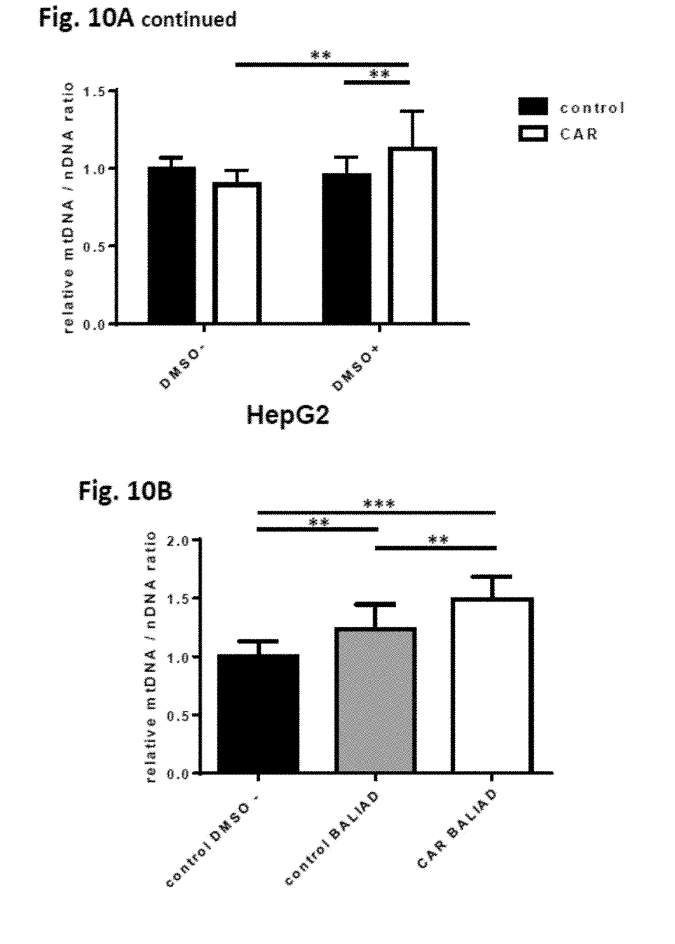

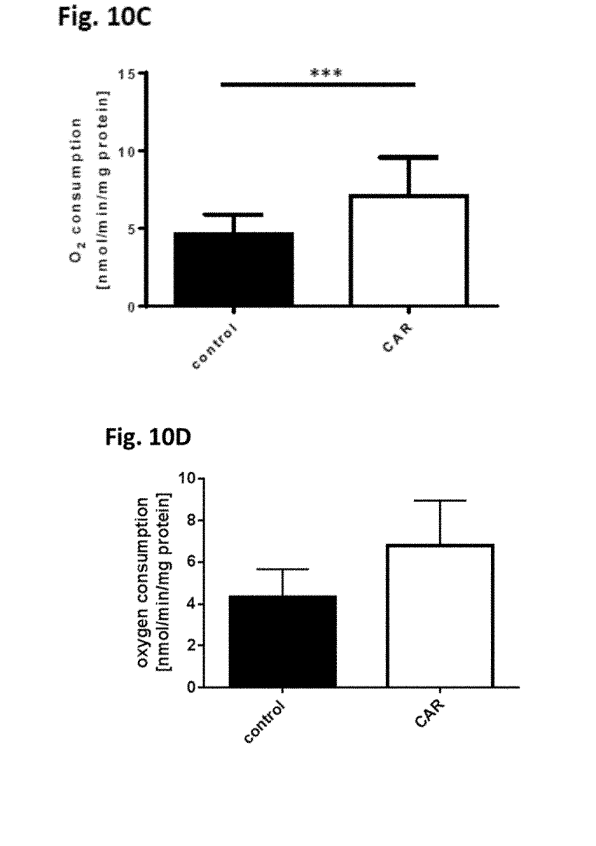

Despite a trend towards lower glucose consumption and less lactate production and higher WST activity, total cellular ATP content was unaltered by CAR overexpression in HepaRG cells (FIG. 8C). Increased oxidative phosphorylation in mitochondria of HepaRG-CAR might provide an explanation for the observed reduction in glycolytic activity. Culturing HepaRG and HepaRG-CAR cells increased the mitochondrial content, as assessed by the mitochondrial to nuclear DNA ratio, when compared with cells not cultured in presence of DMSO by 1.8× (unmodified HepaRG cells) to 2× (HepaRG-CAR cells) (FIG. 10A). When cultured both in presence of DMSO, modified HepaRG cells, displayed a higher mitochondrial content, when compared with control cells by 1.4× (FIG. 10A). In HepG2 cells the effect of CAR overexpression on mitochondrial content was also found. In HepG2-CAR cells cultured with DMSO there was a small, but significant, increase when compared to HepG2-CAR cells cultured without DMSO (1.3×) or when compared to HepG2 control cells cultured with DMSO (1.2×) (FIG. 10A). In HepaRG cells BALIAD culture increased the mitochondrial to nuclear DNA ratio by 6× compared to control monolayer culture, while BALIAD culture of modified HepaRG cells did not further change this ratio (FIG. 10B). Furthermore, oxygen consumption in modified HepaRG cells cultured in monolayers and in BALIADs was 1.5× to 2× higher when compared with control cells (FIGS. 10C and D). These data suggest that CAR overexpression reduces glycolytic activity in favor of oxidative phosphorylation for energy supply in HepaRG cells.

Increased Survival of Modified HepaRG Cells During Long-Term Culture without FBS

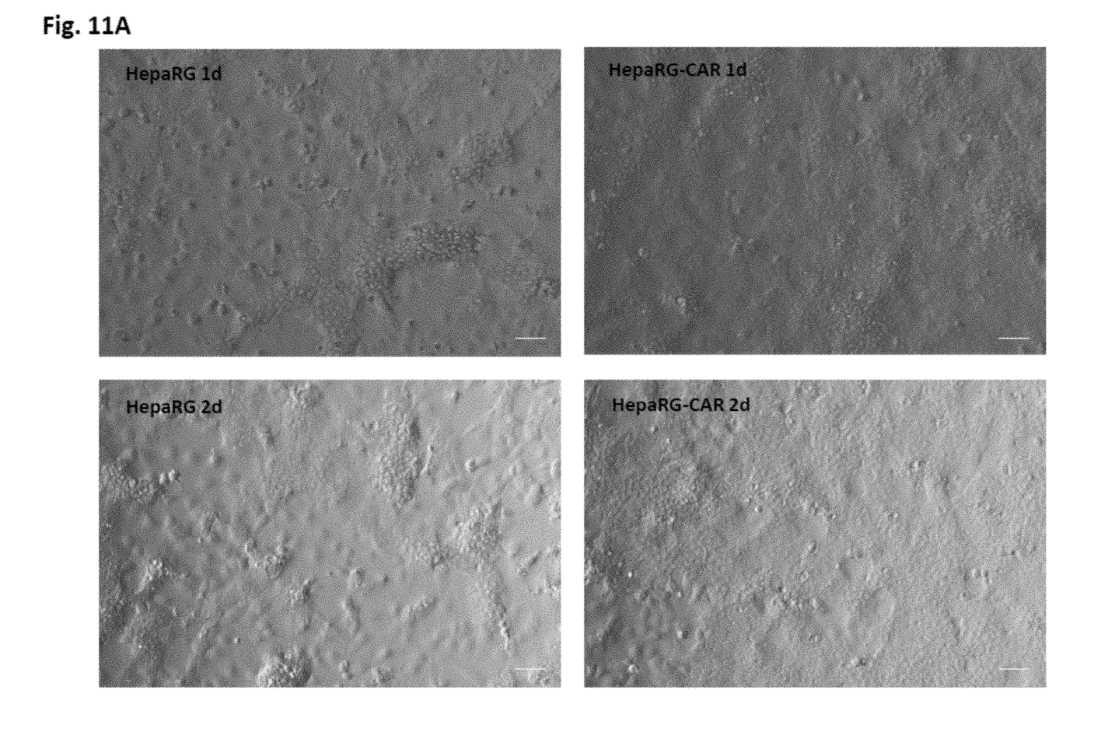

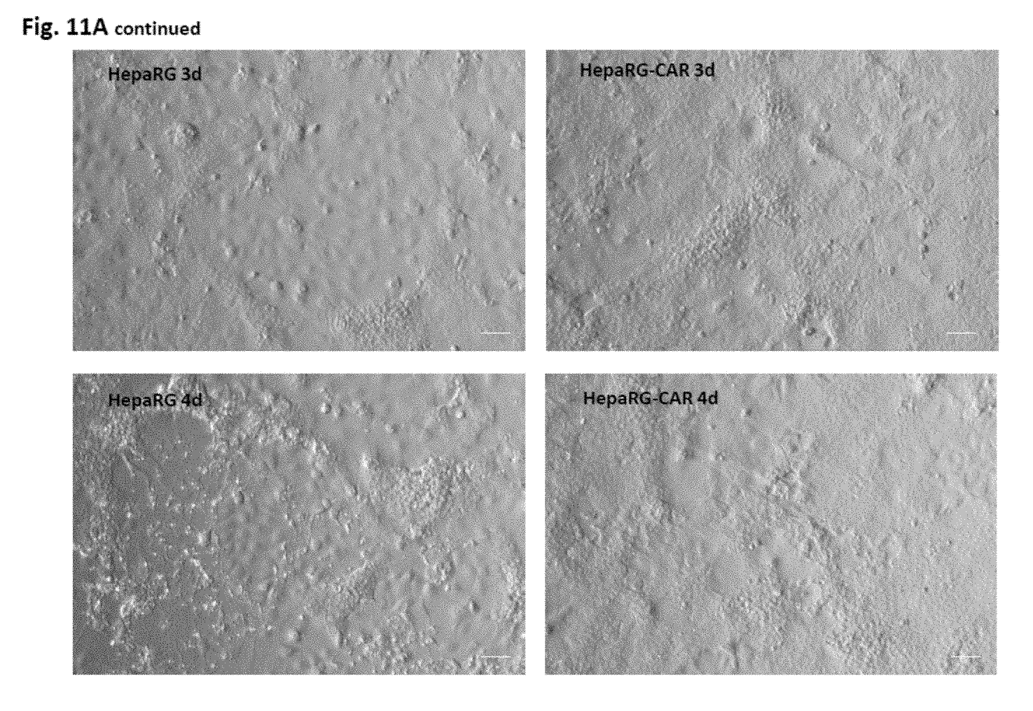

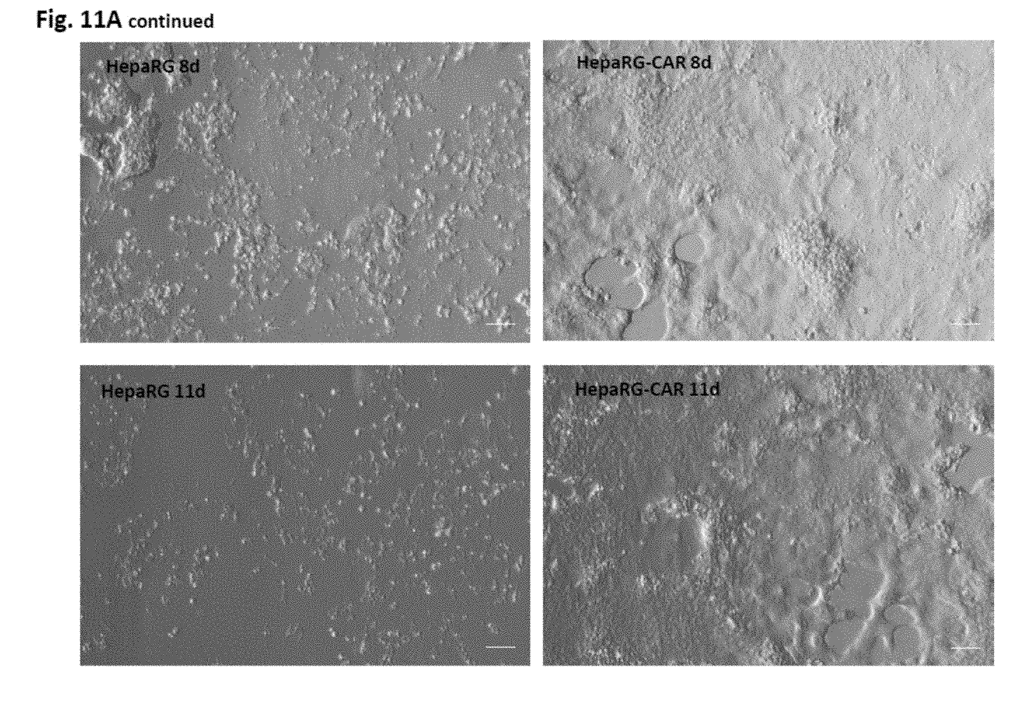

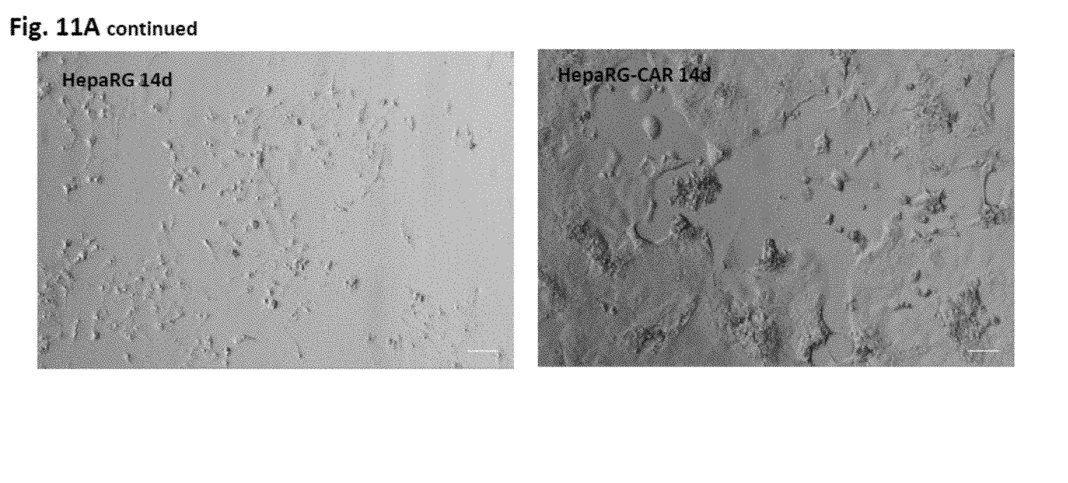

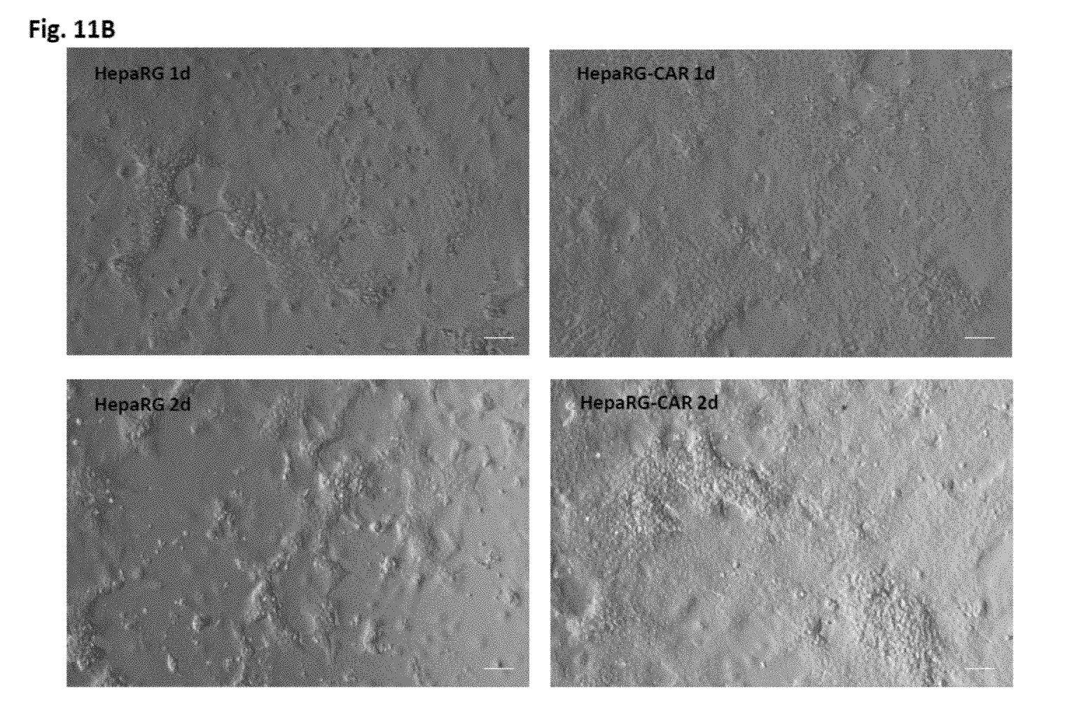

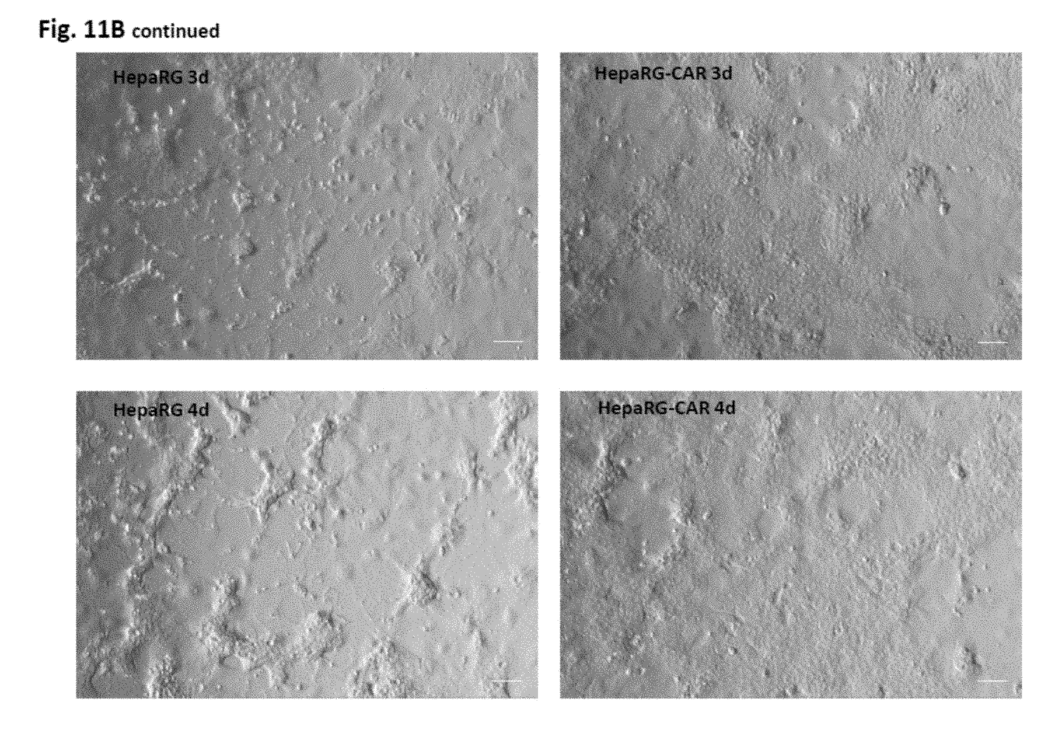

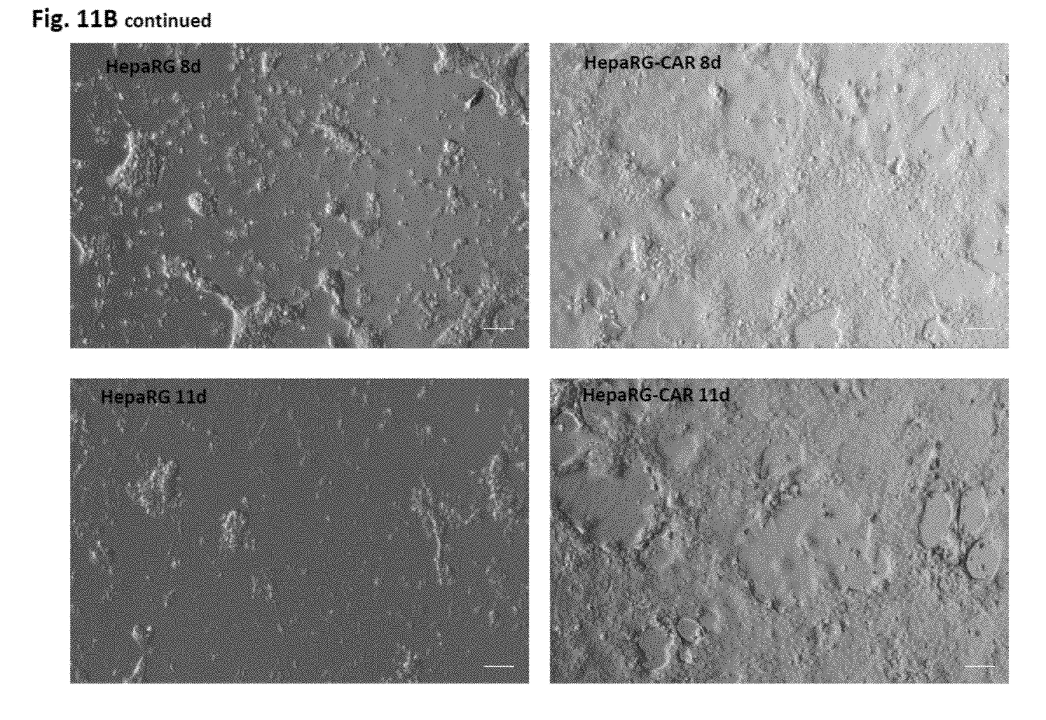

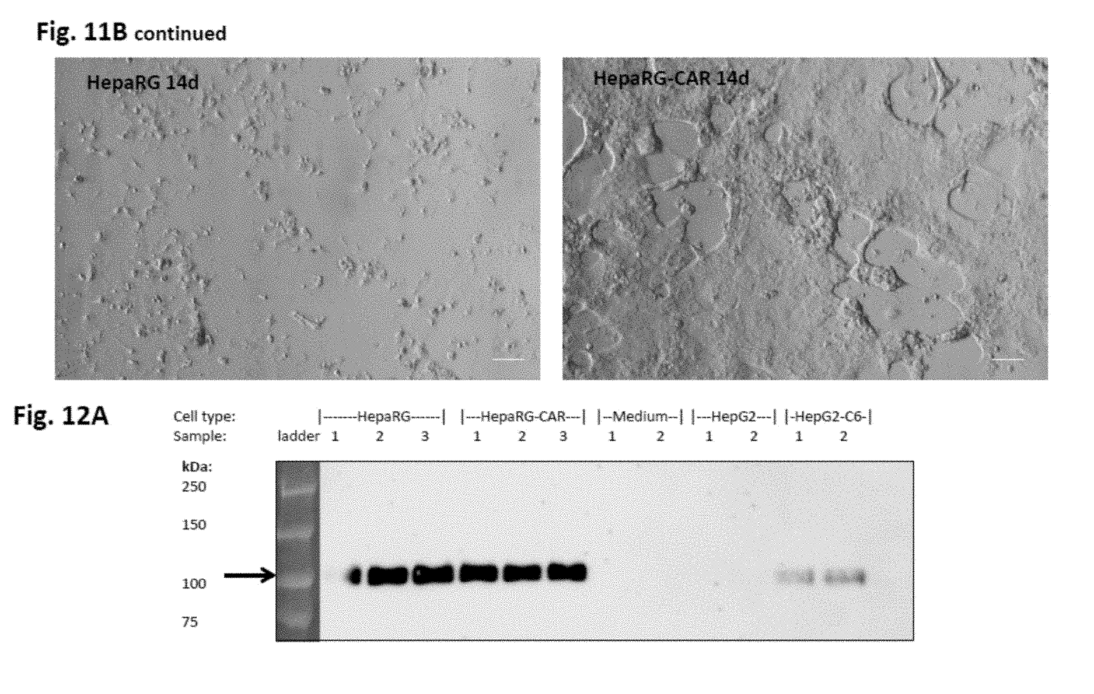

To assess the effect of CAR overexpression on fasting, the inventors studied the morphology of mature HepaRG-CAR and control cells in monolayers maintained in serum-free medium with or without DMSO after the conventional culture of 28 days. Cholangiocytes of control cultures without DMSO were in the process of disappearing after 4 days, with hepatocyte islands disappearing between 8-11 days. Most of the cholangiocytes in modified HepaRG cells cultured without DMSO disappeared between day 4 and 8 (FIG. 11A). However, hepatocyte islands and hepatocyte-like cells remained viable at day 11. Even at day 14 hepatocyte islands were still present (FIG. 11A), although there was a tendency of reduced confluency. modified HepaRG cells cultured with DMSO seemed to be even more resistant to serum-free culture conditions (FIG. 11B). Cholangiocytes in control cultures became necrotic at day 2 to 3 after removal of FBS, while hepatocyte islands were disappearing between day 4 and 8. The small amount of cholangiocytes in modified HepaRG cells started to disappear from around day 8, while hepatocyte-like cells and hepatocyte islands appeared to be viable even at day 14 (FIG. 11B). Thus, CAR overexpression renders the HepaRG cells highly resistant to FBS depletion.

Reduced Mitochondrial Superoxide Levels in Modified HepaRG Cells

The inventors studied the mitochondrial superoxide levels, hypothesizing that these may have decreased as a result of the changed energy metabolism and mitochondrial functionality in modified HepaRG cells. Indeed, modified HepaRG cells from monolayers cultured without DMSO contained a 2-fold lower content of mitochondrial superoxide compared to similar cultures of unmodified HepaRG cells (FIG. 11). This difference in superoxide levels may have substantial effects, as superoxide has damaging effects, and also has a signaling function (Forkink, Smeikink, 2010).

HepaRG Cells Cultured in BALIAD Produce High Levels of Fibrinogen and Complement Factor 6

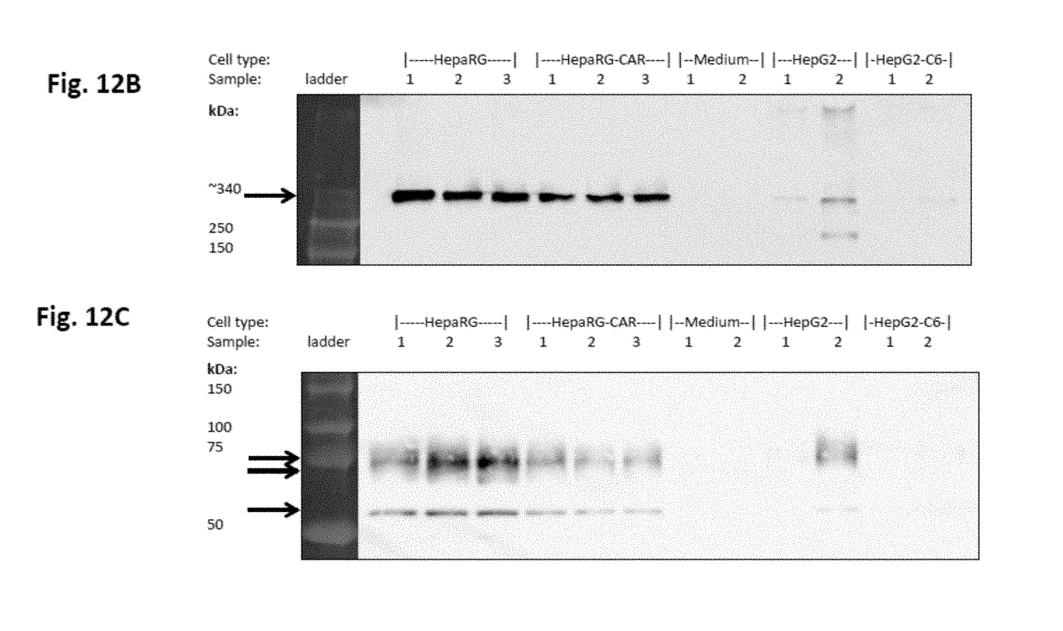

The inventors assessed the capability of the modified HepaRG cells in BALIADs to produce two important blood proteins during 3 days in FBS-free conditions: complement factor 6 (C6) and fibrinogen. In comparison with monolayer cultures of HepG2 cells or HepG2 cells overexpressing C6 (HepG2-C6), both HepaRG and modified HepaRG cells secreted a large amount of C6 (FIG. 12A). In addition, fibrinogen secretion was also high in HepaRG and modified HepaRG cells (FIG. 12B+C). These results, combined with the improved survival in FBS-free culture conditions, show that the modified HepaRG cells of the invention are a suitable production cell line for liver cell-derived blood proteins in serum-free culture medium.

Increased Infection of Modified HepaRG Cells by Plasmodium falciparum

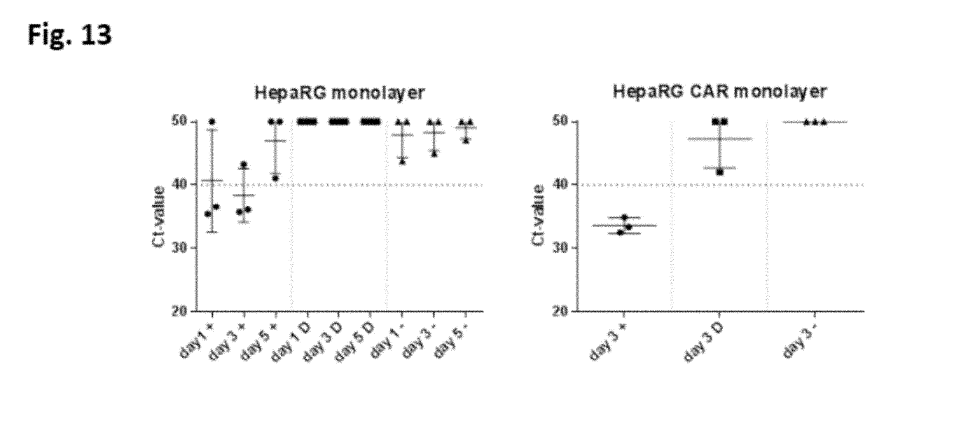

Because of the improved metabolic state of modified HepaRG cells, the inventors assessed infection of P. falciparum in HepaRG+/− CAR cells cultured in monolayer without DMSO. Interestingly, the inventors observed increased levels of P. falciparum 18S ribosomal RNA (28×) 3 days after infection of modified HepaRG cells compared to normal HepaRG cells (FIG. 13), indicating a possible use for modified HepaRG cells in studies on the liver stage of human malaria infections.

Increased Stability at Serial Passaging of Modified HepaRG Cells

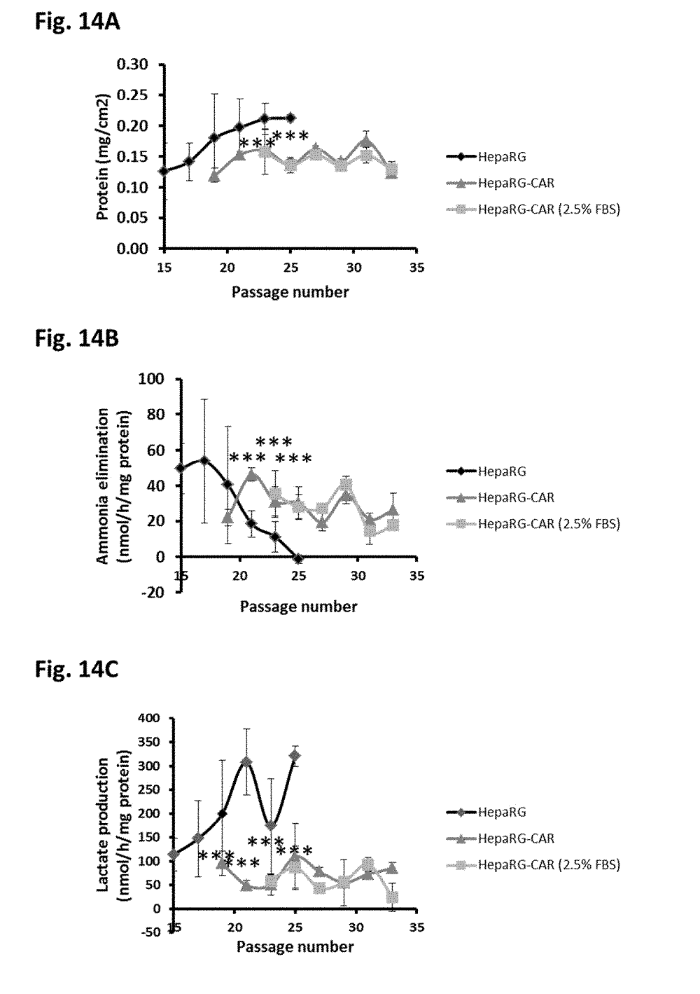

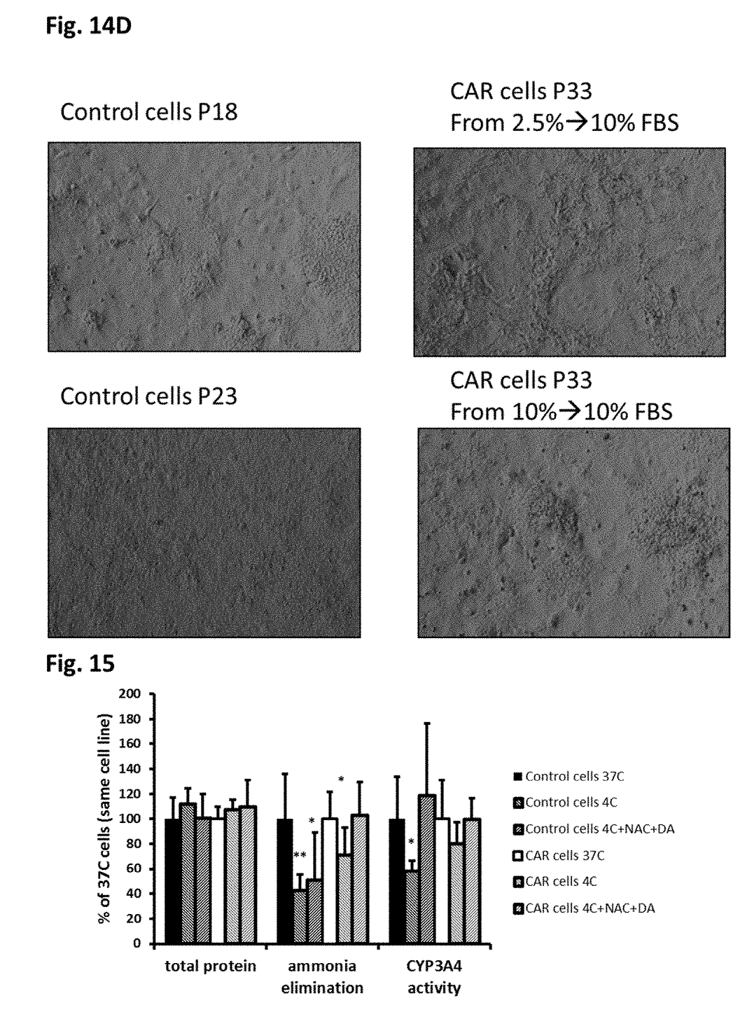

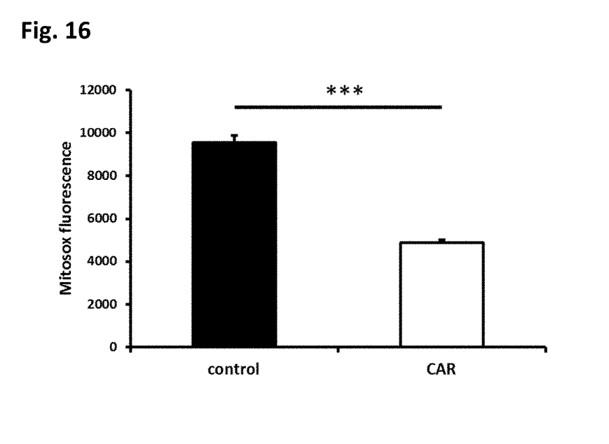

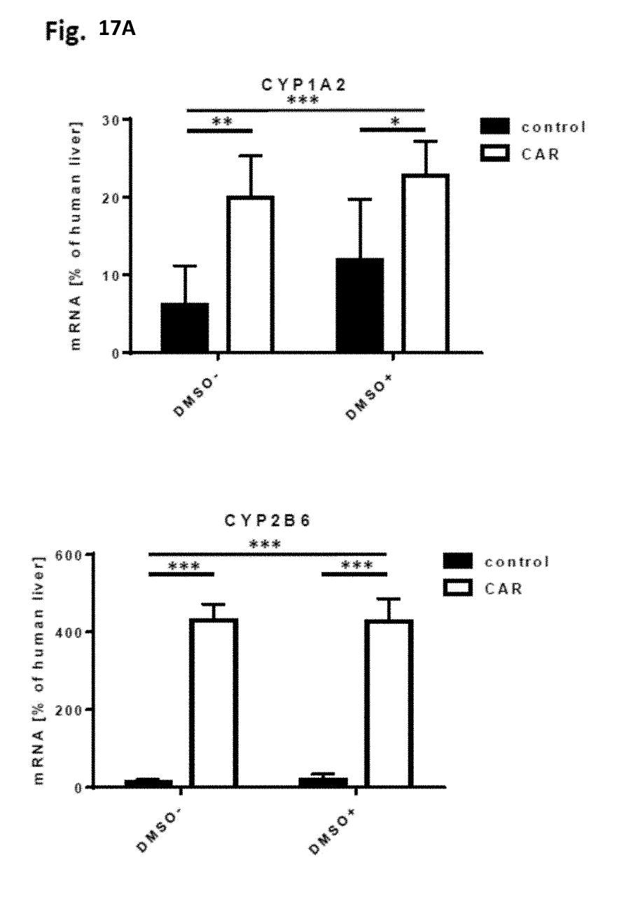

The inventors compared also the effects of serial passaging on unmodified and modified HepaRG cells. Since the metabolic state of modified HepaRG cells was improved, and the mitochondrial superoxide levels were reduced, it was hypothesized that the stability of HepaRG cells might be increased due to CAR overexpression. In unmodified HepaRG cells, the cells started to increase proliferation after critical passage 20 (Laurent, Glaise et al. 2013), leading to increased protein levels, the loss of characteristic morphology of terminally differentiated monolayer cultures showing hepatocyte islands surrounded by less differentiated flat cells, a decrease of ammonia elimination and and increase of lactate production, leading to high acidification of the culture medium (FIG. 14 A-D). In contrast, modified HepaRG cells, whether maintained in standard HepaRG culture medium with 10% FBS or in 2.5% FBS, maintained their phenotype, at least up to passage 33. This indicates that modified HepaRG cells are highly stable and can be expanded to large cell masses, which is highly desirable for future applications.

Increased Resistance to Hypothermic Preservation of Modified HepaRG Cells

As modified HepaRG cells appeared to be more robust, the inventors also studied the effects of a 24-hour preservation period at 4° C. on BALIAD cultures of HepaRG and modified HepaRG cells. The hypothermic preserved cells were optionally treated with antioxidants NAC and DA, which have previously been described to have a protective effect in hypothermic preservation of hepatocytes or liver (Gómez-Lechón, Lahoz et al. 2008; Risso, Koike et al. 2014, Koetting, Stegeman et al. 2010, Minor, Luer et al. 2011). There was no effect of the hypothermic preservation on total protein content of the cultures. However, the ammonia elimination was reduced 58% in unmodified HepaRG cells and treatment with NAC+DA could not reverse this negative effect (FIG. 15). In addition, the CYP3A4 activity was reduced 50%, which could, however be counteracted by the NAC+DA treatment. The modified HepaRG cells were less susceptible to the negative effects of hypothermic preservation; the ammonia elimination was only 29% reduced and was completely reversed by NAC+DA treatment and CYP3A4 activity was not affected by the hypothermic preservation at all. This indicates that highly differentiated modified HepaRG cells in BALIADs may be completely protected against a hypothermic preservation procedure, which highly facilitates the transport of large cell masses, for instance in a bioartificial liver.

### REFERENCES

Andersson, T. B., Kanebratt, K. P., Kenna, J. G., 2012. The HepaRG cell line: a unique in vitro tool for understanding drug metabolism and toxicology in human. Expert opinion on drug metabolism & toxicology 8, 909-920.
Aninat, C., Piton, A., Glaise, D., Le Charpentier, T., Langouet, S., Morel, F., Guguen-Guillouzo, C., Guillouzo, A., 2006. Expression of cytochromes P450, conjugating enzymes and nuclear receptors in human hepatoma HepaRG cells. Drug metabolism and disposition: the biological fate of chemicals 34, 75-83.
Azuma, H., Hirose, T., Fujii, H., Oe, S., Yasuchika, K., Fujikawa, T., Yamaoka, Y., 2003. Enrichment of hepatic progenitor cells from adult mouse liver. Hepatology 37, 1385-1394.
Baes, M., Gulick, T., Choi, H. S., Martinoli, M. G., Simha, D., Moore, D. D., 1994. A new orphan member of the nuclear hormone receptor superfamily that interacts with a subset of retinoic acid response elements. Mol Cell Biol 14, 1544-1552.
Berridge, M. V., Herst, P. M., Tan, A. S., 2005. Tetrazolium dyes as tools in cell biology: new insights into their cellular reduction. Biotechnol Annu Rev 11, 127-152.
Bonn, B., Svanberg, P., Janefeldt, A., Hultman, I., Grime, K., 2016. Determination of Human Hepatocyte Intrinsic Clearance for Slowly Metabolized Compounds: Comparison of a Primary Hepatocyte/Stromal Cell Co-culture with Plated Primary Hepatocytes and HepaRG. Drug Metab Dispos 44, 527-533.
Busby, W. F., Jr., Ackermann, J. M., Crespi, C. L., 1999. Effect of methanol, ethanol, dimethyl sulfoxide, and acetonitrile on in vitro activities of cDNA-expressed human cytochromes P-450. Drug Metab Dispos 27, 246-249.
Cerec, V., Glaise, D., Garnier, D., Morosan, S., Turlin, B., Drenou, B., Gripon, P., Kremsdorf, D., Guguen-Guillouzo, C., Corlu, A., 2007. Transdifferentiation of hepatocyte-like cells from the human hepatoma HepaRG cell line through bipotent progenitor. Hepatology (Baltimore, Md.) 45, 957-967.
Chan, T. S., Yu, H., Moore, A., Kehtani, S. R., Tweedie, D., 2013. Meeting the Challenge of Predicting Hepatic Clearance of Compounds Slowly Metabolized by Cytochrome P450 Using a Novel Hepatocyte Model, HepatoPac. Drug metabolism and disposition: the biological fate of chemicals 41, 2024-2032.
Chauret, N., Gauthier, A., Nicoll-Griffith, D. A., 1998. Effect of common organic solvents on in vitro cytochrome P450-mediated metabolic activities in human liver microsomes. Drug Metab Dispos 26, 1-4.
Choi, H. S., Chung, M., Tzameli, I., Simha, D., Lee, Y. K., Seol, W., Moore, D. D., 1997. Differential transactivation by two isoforms of the orphan nuclear hormone receptor CAR. J Biol Chem 272, 23565-23571.
Choi, S., Sainz, B., Corcoran, P., Uprichard, S., Jeong, H., 2009. Characterization of increased drug metabolism activity in dimethyl sulfoxide (DMSO)-treated Huh7 hepatoma cells. Xenobiotica; the fate of foreign compounds in biological systems 39, 205-217.
Dembele, L., Franetich, J. F., Lorthiois, A., Gego, A., Zeeman, A. M., Kocken, C. H., Le Grand, R., Dereuddre-Bosquet, N., van Gemert, G. J., Sauerwein, R., Vaillant, J. C., Hannoun, L., Fuchter, M. J., Diagana, T. T., Malmquist, N. A., Scherf, A., Snounou, G., Mazier, D., 2014. Persistence and activation of malaria hypnozoites in long-term primary hepatocyte cultures. Nat Med 20, 307-312.
Di, L., Atkinson, K., Orozco, C. C., Funk, C., Zhang, H., McDonald, T. S., Tan, B., Lin, J., Chang, C., Obach, R. S., 2013. In vitro-in vivo correlation for low-clearance compounds using hepatocyte relay method. Drug metabolism and disposition: the biological fate of chemicals 41, 2018-2023.
Dull, T., Zufferey, R., Kelly, M., Mandel, R. J., Nguyen, M., Trono, D., Naldini, L., 1998. A third-generation lentivirus vector with a conditional packaging system. J Virol 72, 8463-8471.
Flendrig, L. M., la Soe, J. W., Jorning, G. G., Steenbeek, A., Karlsen, O. T., Bovee, W. M., Ladiges, N. C., to Velde, A. A., Chamuleau, R. A., 1997. In vitro evaluation of a novel bioreactor based on an integral oxygenator and a spirally wound nonwoven polyester matrix for hepatocyte culture as small aggregates. J Hepatol 26, 1379-1392.
Forkink M., Smeitink J. A., Brock R., Willems P. H., Koopman W. J., 2010. Detection and manipulation of mitochondrial reactive oxygen species in mammalian cells. Biochim Biophys Acta, 1797, 1034-1044.
Galvao, J., Davis, B., Tilley, M., Normando, E., Duchen, M. R., Cordeiro, M. F., 2014. Unexpected low-dose toxicity of the universal solvent DMSO. FASEB J 28, 1317-1330.
Gao, J., Xie, W., 2010. Pregnane X receptor and constitutive androstane receptor at the crossroads of drug metabolism and energy metabolism. Drug Metab Dispos 38, 2091-2095.
Gomez-Lechón M. J., Lahoz A., Jiménez N., Bonora A., Castell J. V., Donato M. T., 2008.
Evaluation of drug-metabolizing and functional competence of human hepatocytes incubated under hypothermia in different media for clinical infusion. Cell Transplant, 17, 887-897.
Gonzalez-Perez, V., Connolly, E. A., Bridges, A. S., Wienkers, L. C., Paine, M. F., 2012. Impact of organic solvents on cytochrome P450 probe reactions: filling the gap with (S)-Warfarin and midazolam hydroxylation. Drug Metab Dispos 40, 2136-2142.
Gripon, P., Rumin, S., Urban S., Le Seyec J., Glaise D., Cannie I., Guyomard C., Lucas J., Trepo C., Guguen-Guillouzo C., 2002. Infection of a human hepatoma cell line by hepatitis B virus. Proc Natl Acad Sci USA., 99, 15655-15660.
Hernandez, J. P., Mota, L. C., Baldwin, W. S., 2009. Activation of CAR and PXR by Dietary, Environmental and Occupational Chemicals Alters Drug Metabolism, Intermediary Metabolism, and Cell Proliferation. Curr Pharmacogenomics Person Med 7, 81-105.
Higgins, P. J., Borenfreund, E., 1980. Enhanced albumin production by malignantly transformed hepatocytes during in vitro exposure to dimethylsulfoxide. Biochim Biophys Acta 610, 174-180.
Hirschhaeuser, F., Sattler, U. G., Mueller-Klieser, W., 2011. Lactate: a metabolic key player in cancer. Cancer Res 71, 6921-6925.
Hoekstra, R., Deurholt, T., Poyck, P. P., ten Bloemendaal, L., Chhatta, A. A., 2005. Increased reproducibility of quantitative reverse transcriptase-PCR. Anal Biochem 340, 376-379.
Hoekstra, R., Nibourg, G. A., van der Hoeven, T. V., Ackermans, M. T., Hakvoort, T. B., van Gulik, T. M., Lamers, W. H., Elferink, R. P., Chamuleau, R. A., 2011. The HepaRG cell line is suitable for bioartificial liver application. Int J Biochem Cell Biol 43, 1483-1489.
Huang, W., Zhang, J., Moore, D. D., 2004. A traditional herbal medicine enhances bilirubin clearance by activating the nuclear receptor CAR. The Journal of clinical investigation 113, 137-143.
Hutter, B., John, G. T., 2004. Evaluation of OxoPlate for real-time assessment of antibacterial activities. Curr Microbiol 48, 57-61.
Hutzler, J. M., Ring, B. J., Anderson, S. R., 2015. Low-Turnover Drug Molecules: A Current Challenge for Drug Metabolism Scientists. Drug Metab Dispos 43, 1917-1928.
Jennen, D. G., Magkoufopoulou, C., Ketelslegers, H. B., van Herwijnen, M. H., Kleinjans, J. C., van Delft, J. H., 2010. Comparison of HepG2 and HepaRG by whole-genome gene expression analysis for the purpose of chemical hazard identification. Toxicological sciences: an official journal of the Society of Toxicology 115, 66-79.
John, G. T., Klimant, I., Wittmann, C., Heinzle, E., 2003. Integrated optical sensing of dissolved oxygen in microtiter plates: a novel tool for microbial cultivation. Biotechnol Bioeng 81, 829-836.
Kanebratt, K. P., Andersson, T. B., 2008a. Evaluation of HepaRG cells as an in vitro model for human drug metabolism studies. Drug metabolism and disposition: the biological fate of chemicals 36, 1444-1452.
Kanebratt, K. P., Andersson, T. B., 2008b. HepaRG cells as an in vitro model for evaluation of cytochrome P450 induction in humans. Drug metabolism and disposition: the biological fate of chemicals 36, 137-145.
Kawamoto, T., Sueyoshi, T., Zelko, I., Moore, R., Washburn, K., Negishi, M., 1999. Phenobarbital-responsive nuclear translocation of the receptor CAR in induction of the CYP2B gene. Mol Cell Biol 19, 6318-6322.
Knox, W. E., Mahler, A. H., 1951. The adaptive increase of the tryptophan peroxidase-oxidase system of liver. Science 113, 237-238.
Koetting M., Stegemann J., Minor T., 2010. Dopamine as additive to cold preservation solution improves postischemic integrity of the liver. Transpl Int, 23, 951-958.
Laurent V., Glaise D., Nübel T., Gilot D., Corlu A., Loyer P., 2013. Highly efficient SiRNA and gene transfer into hepatocyte-like HepaRG cells and primary human hepatocytes: new means for drug metabolism and toxicity studies. Methods Mol Biol 987, 295-314.
Li, D., Mackowiak, B., Brayman, T. G., Mitchell, M., Zhang, L., Huang, S. M., Wang, H., 2015. Genome-wide analysis of human constitutive androstane receptor (CAR) transcriptome in wild-type and CAR-knockout HepaRG cells. Biochem Pharmacol. 98, 190-202.
Li, H., Chen, T., Cottrell, J., Wang, H., 2009. Nuclear translocation of adenoviral-enhanced yellow fluorescent protein-tagged-human constitutive androstane receptor (hCAR): a novel tool for screening hCAR activators in human primary hepatocytes. Drug Metab Dispos 37, 1098-1106.
Liao, M., Pabarcus, M. K., Wang, Y., Hefner, C., Maltby, D. A., Medzihradszky, K. F., Salas-Castillo, S. P., Yan, J., Maher, J. J., Correia, M. A., 2007. Impaired dexamethasone-mediated induction of tryptophan 2,3-dioxygenase in heme-deficient rat hepatocytes: translational control by a hepatic eIF2alpha kinase, the heme-regulated inhibitor. J Pharmacol Exp Ther 323, 979-989.
Lynch, C., Zhao, J., Huang, R., Xiao, J., Li, L., Heyward, S., Xia, M., Wang, H., 2015. Quantitative high-throughput identification of drugs as modulators of human constitutive androstane receptor. Sci Rep 5, 10405.
Maglich, J. M., Watson, J., McMillen, P. J., Goodwin, B., Willson, T. M., Moore, J. T., 2004. The nuclear receptor CAR is a regulator of thyroid hormone metabolism during caloric restriction. J Biol Chem 279, 19832-19838.
March, S., Ng, S., Velmurugan, S., Galstian, A., Shan, J., Logan, D. J., Carpenter, A. E., Thomas, D., Sim, B. K., Mota, M. M., Hoffman, S. L., Bhatia, S. N., 2013. A microscale human liver platform that supports the hepatic stages of Plasmodium falciparum and vivax. Cell Host Microbe 14, 104-115.
March, S., Ramanan, V., Trehan, K., Ng, S., Galstian, A., Gural, N., Scull, M. A., Shlomai, A., Mota, M. M., Fleming, H. E., Khetani, S. R., Rice, C. M., Bhatia, S. N., 2015. Micropatterned coculture of primary human hepatocytes and supportive cells for the study of hepatotropic pathogens. Nat Protoc 10, 2027-2053.
McGill, M. R., Sharpe, M. R., Williams, C. D., Taha, M., Curry, S. C., Jaeschke, H., 2012. The mechanism underlying acetaminophen-induced hepatotoxicity in humans and mice involves mitochondrial damage and nuclear DNA fragmentation. The Journal of clinical investigation 122, 1574-1583.
Mikolajczak, S. A., Vaughan, A. M., Kangwanrangsan, N., Roobsoong, W., Fishbaugher, M., Yimamnuaychok, N., Rezakhani, N., Lakshmanan, V., Singh, N., Kaushansky, A., Camargo, N., Baldwin, M., Lindner, S. E., Adams, J. H., Sattabongkot, J., Kappe, S. H., 2015. Plasmodium vivax liver stage development and hypnozoite persistence in human liver-chimeric mice. Cell Host Microbe 17, 526-535.
Minor T., Luer B., 2011. Efferz P., Dopamine improves hypothermic machine preservation of the liver. Cryobiology, 63, 84-89
Molnár, F., Küblbeck, J., Jyrkkärinne, J., Prantner, V., Honkakoski, P., 2013. An update on the constitutive androstane receptor (CAR). Drug metabolism and drug interactions 28, 79-93.
Mutoh, S., Sobhany, M., Moore, R., Perera, L., Pedersen, L., Sueyoshi, T., Negishi, M., 2013. Phenobarbital Indirectly Activates the Constitutive Active Androstane Receptor (CAR) by Inhibition of Epidermal Growth Factor Receptor Signaling. Science Signaling 6.
Nibourg, G. A., Chamuleau, R. A., van der Hoeven, T. V., Maas, M. A., Ruiter, A. F., Lamers, W. H., Oude Elferink, R. P., van Gulik, T. M., Hoekstra, R., 2012a. Liver progenitor cell line HepaRG differentiated in a bioartificial liver effectively supplies liver support to rats with acute liver failure. PLoS One 7, e38778.
Nibourg, G. A., Chamuleau, R. A., van Gulik, T. M., Hoekstra, R., 2012b. Proliferative human cell sources applied as biocomponent in bioartificial livers: a review. Expert Opin Biol Ther 12, 905-921.
Nibourg, G. A., Huisman, M. T., van der Hoeven, T. V., van Gulik, T. M., Chamuleau, R. A., Hoekstra, R., 2010. Stable overexpression of pregnane X receptor in HepG2 cells increases its potential for bioartificial liver application. Liver Transpl 16, 1075-1085.
Nibourg, G. A. A., Hoekstra, R., van der Hoeven, T. V., Ackermans, M. T., Hakvoort, T. B., van Gulik, T. M., Chamuleau, R. A., 2013. Increased hepatic functionality of the human hepatoma cell line HepaRG cultured in the AMC bioreactor. The international journal of biochemistry & cell biology 45, 1860-1868.
Paran, N., Geiger, B., Shaul, Y., 2001. HBV infection of cell culture: evidence for multivalent and cooperative attachment. EMBO J 20, 4443-4453.
Paulusma, C. C., Bosma, P. J., Zaman, G. J., Bakker, C. T., Otter, M., Scheffer, G. L., Scheper, R. J., Borst, P., Oude Elferink, R. P., 1996. Congenital jaundice in rats with a mutation in a multidrug resistance-associated protein gene. Science 271, 1126-1128.
Prudencio, M., Rodriguez, A., Mota, M. M., 2006. The silent path to thousands of merozoites: the Plasmodium liver stage. Nat Rev Microbiol 4, 849-856.
Ramaiahgari, S. C., den Braver, M. W., Herpers, B., Terpstra, V., Cornmandeur, J. N., van de Water, B., Price, L. S., 2014. A 3D in vitro model of differentiated HepG2 cell spheroids with improved liver-like properties for repeated dose high-throughput toxicity studies. Arch Toxicol 88, 1083-1095.
Risso P. S., Koike M. K., Abrahão Mde S., Ferreira N. C., Montero E. F., 2014. The effect of n-acetylcysteine on hepatic histomorphology during hypothermic preservation. Acta Cir Bras. 29 Suppl 3, 28-32.
Robanus-Maandag, E., Dekker, M., van der Valk, M., Carrozza, M. L., Jeanny, J. C., Dannenberg, J. H., Berns, A., to Riele, H., 1998. p 107 is a suppressor of retinoblastoma development in pRb-deficient mice. Genes Dev 12, 1599-1609.
Ruijter, J. M., Ramakers, C., Hoogaars, W. M., Karlen, Y., Bakker, O., van den Hoff, M. J., Moorman, A. F., 2009. Amplification efficiency: linking baseline and bias in the analysis of quantitative PCR data. Nucleic Acids Res 37, e45.
Sainz, B., Jr., Chisari, F. V., 2006. Production of infectious hepatitis C virus by well-differentiated, growth-arrested human hepatoma-derived cells. J Virol 80, 10253-10257.
Sattabongkot, J., Yimamnuaychoke, N., Leelaudomlipi, S., Rasameesoraj, M., Jenwithisuk, R., Coleman, R. E., Udomsangpetch, R., Cui, L., Brewer, T. G., 2006. Establishment of a human hepatocyte line that supports in vitro development of the exo-erythrocytic stages of the malaria parasites Plasmodium falciparum and P. vivax. Am J Trop Med Hyg 74, 708-715.
Seppen, J., Rijnberg, M., Cooreman, M. P., Oude Elferink, R. P., 2002. Lentiviral vectors for efficient transduction of isolated primary quiescent hepatocytes. J Hepatol 36, 459-465.
Soulard, V., Bosson-Vanga, H., Lorthiois, A., Roucher, C., Franetich, J. F., Zanghi, G., Bordessoulles, M., Tefit, M., Thellier, M., Morosan, S., Le Naour, G., Capron, F., Suemizu, H., Snounou, G., Moreno-Sabater, A., Mazier, D., 2015. Plasmodium falciparum full life cycle and Plasmodium ovale liver stages in humanized mice. Nat Commun 6, 7690.
Spivak, W., Carey, M. C., 1985. Reverse-phase h.p.l.c. separation, quantification and preparation of bilirubin and its conjugates from native bile. Quantitative analysis of the intact tetrapyrroles based on h.p.l.c. of their ethyl anthranilate azo derivatives. Biochem J 225, 787-805.
Sturm, A., Amino, R., van de Sand, C., Regen, T., Retzlaff, S., Rennenberg, A., Krueger, A., Pollok, J. M., Menard, R., Heussler, V. T., 2006. Manipulation of host hepatocytes by the malaria parasite for delivery into liver sinusoids. Science 313, 1287-1290.
Tirona, R. G., Lee, W., Leake, B. F., Lan, L.-B. B., Cline, C. B., Lamba, V., Parviz, F., Duncan, S. A., Inoue, Y., Gonzalez, F. J., Schuetz, E. G., Kim, R. B., 2003. The orphan nuclear receptor HNF4alpha determines PXR- and CAR-mediated xenobiotic induction of CYP3A4. Nature medicine 9, 220-224.
van Wenum, M., Chamuleau, R. A., van Gulik, T. M., Siliakus, A., Seppen, J., Hoekstra, R., 2014. Bioartificial livers in vitro and in vivo: tailoring biocomponents to the expanding variety of applications. Expert Opin Biol Ther 14, 1745-1760.
Vander Heiden, M. G., Cantley, L. C., Thompson, C. B., 2009. Understanding the Warburg effect: the metabolic requirements of cell proliferation. Science 324, 1029-1033.
Villa, P., Arioli, P., Guaitani, A., 1991. Mechanism of maintenance of liver-specific functions by DMSO in cultured rat hepatocytes. Exp Cell Res 194, 157-160.
Watanabe, Y., Fujiwara, M., Yoshida, R., Hayaishi, O., 1980. Stereospecificity of hepatic L-tryptophan 2,3-dioxygenase. Biochem J 189, 393-405.
Xu, J. J., Henstock, P. V., Dunn, M. C., Smith, A. R., Chabot, J. R., de Graaf, D., 2008. Cellular imaging predictions of clinical drug-induced liver injury. Toxicol Sci 105, 97-105.
Yang, H., Wang, H., 2014. Signaling control of the constitutive androstane receptor (CAR). Protein & cell 5, 113-123.
Yu, Z. W., Quinn, P. J., 1994. Dimethyl sulphoxide: a review of its applications in cell biology. Biosci Rep 14, 259-281.
Zanelli, U., Caradonna, N. P., Hallifax, D., Turlizzi, E., Houston, J. B., 2012. Comparison of cryopreserved HepaRG cells with cryopreserved human hepatocytes for prediction of clearance for 26 drugs. Drug metabolism and disposition: the biological fate of chemicals 40, 104-110.
Zufferey, R., Dull, T., Mandel, R. J., Bukovsky, A., Quiroz, D., Naldini, L., Trono, D., 1998. Self-inactivating lentivirus vector for safe and efficient in vivo gene delivery. J Virol 72, 9873-9880.

1. A modified HepaRG cell, wherein the modification comprises an additional copy of the CAR/NR1I3 gene compared to the HepaRG cell as deposited on 5 Apr. 2001 at the Collection Nationale de Cultures de Microorganismes, Institut Pasteur, under No. 1-2652.

2. The modified HepaRG cell according to claim 1, wherein the modification induces overexpression of the CAR/NR1I3 gene in comparison with the HepaRG cell as deposited on 5 Apr. 2001 at the Collection Nationale de Cultures de Microorganismes, Institut Pasteur, under No. 1-2652.

3. The modified HepaRG cell as deposited on Oct. 5, 2016 at the Leibniz-Institut DSMZ Deutsche Sammlung von Mikroorganismen and Zellkulturen GmbH, under No. DSM ACC3291.

4. Method of producing the modified HepaRG cell according claim 1, comprising:
a. providing a cell culture of HepaRG cells,
b. modifying the HepaRG cells using a nucleic acid construct comprising the CAR/NR1I3 gene and a selection marker, and
c. selecting a modified HepaRG cell using the selection marker.

5. A method of culturing the modified HepaRG cell as defined in claim 1 in a culture medium.

6. The method according to claim 5, wherein the culturing is performed in a three-dimensional culture system, optionally BALIAD.

7. The method according to claim 5, wherein the culture medium is substantially free of DMSO.

8. The method according to claim 5, wherein the culture medium is substantially free of serum.

9. The modified HepaRG cell obtainable by the method according to claim 5.

10. A cell culture comprising the modified HepaRG cell as defined in claim 1.

11. The cell culture according to claim 10, comprising a presence of hepatocyte-like cells in between the hepatocyte islands of more than 25% of the cells in said cell culture, optionally more than 30, 35, 40, 45, 50%.

12. A modified HepaRG cell according to claim 1 capable of being used in a method of determining clearance of a compound.

13. Method of producing a protein of interest comprising:
a. providing a cell culture of modified HepaRG cells according to claim 10,
b. allow the expression of the protein of interest, and
c. isolate the protein of interest.

14. The method according to claim 13, wherein b. is performed in the absence of serum.

15. A method of infecting a modified HepaRG cell with a malaria parasite comprising:
a. providing a cell culture of modified HepaRG cells according to claim 10, and
adding malaria parasites to the cell culture and allow the infection of the modified HepaRG cells.
